(19) United States
(12) Patent Application Publication
(10) Pub. No.: US 2024/0425850 A1
(43) Pub. Date: Dec. 26, 2024

(54) Noncanonical crRNA for Highly Efficient Genome Editing

(71) Applicant: The Brd. of Trustees of the University of Illinois, Urbana, IL (US)

(72) Inventors: Huimin Zhao, Champaign, IL (US); Guanhua Xun, Champaign, IL (US)

(73) Assignee: The Brd. of Trustees of the University of Illinois, Urbana, IL (US)

(21) Appl. No.: 18/680,654

(22) Filed: May 31, 2024

Related U.S. Application Data
(60) Provisional application No. 63/505,413, filed on May 31, 2023.

(51) Int. Cl. C12N 15/11; C12N 9/22; C12N 15/88; C12N 15/90

(52) U.S. Cl. CPC C12N 15/11; C12N 9/22; C12N 15/88; C12N 15/907; C12N 2310/20; C12N 2800/80

(57) ABSTRACT

The CRISPR-Cas12a system is more advantageous than the widely used CRISPR-Cas9 system in terms of specificity and multiplexibility. However, its on-target editing efficiency is typically much lower than that of the CRISPR-Cas9 system. Provided herein are methods and compositions for improvement of Cas12a on-target editing efficiency by incorporating 2-aminoadenine (base Z, which alters canonical Watson-Crick base pairing) into the crRNA to increase the binding affinity between crRNA and its complementary DNA target. The resulting CRISPR-Cas12a (named zCRISPR-Cas12a thereafter) shows an on-target editing efficiency comparable to that of the CRISPR-Cas9 system but with much lower off-target effects than the CRISPR-Cas9 system in mammalian cells. In addition, zCRISPR-Cas12a can be used for precise gene knock-in and highly efficient multiplex genome editing. Overall, the zCRISPR-Cas12a systems are superior to the CRISPR-Cas9 system.

### PRIORITY

This application claims the benefit of U.S. Ser. No. 63/505,413, which was filed on May 31, 2023, and is incorporated by reference herein in its entirety.

### GOVERNMENT SUPPORT

This invention was made with government support under contract numbers DE-SC0018420 awarded by the Department of Energy. The government has certain rights in the invention.

### SEQUENCE LISTING

The instant application contains a Sequence Listing which has been submitted electronically in XML format and is hereby incorporated by reference in its entirety. Said XML copy, created on Sep. 11, 2024, is named 754713_UIUC-053_SL.xml and is 555,684 bytes in size.

### BACKGROUND

The clustered regularly interspaced short palindromic repeat (CRISPR) associated protein (Cas) based system has been used for genome editing and gene regulation in various organisms in the past decade. The most widely used Cas nuclease is derived from Streptococcus pyogenes (SpCas9), which is also the first programmable nuclease with robust activity in eukaryotic cells. Nonetheless, to facilitate the clinical therapeutic application of SpCas9, it is imperative to undergo multiple rounds of gRNA screening to minimize the risk of non-specific cleavage in the genome. In recent years, additional Cas family members with unique advantages over Cas9 have been identified. One notable example is Cas12a, like AsCas12a, an RNA guided class II nuclease derived from Acidaminococcus sp. AsCas12a shows intrinsically higher specificity than SpCas9, which is likely due to its lower tolerance to guide-target mismatches. Cas12a has the capacity to modify multiple genetic elements simultaneously which can be crucial for unraveling and regulating gene interactions and networks in complex cellular functions. However, existing genome engineering tools have limitations concerning the number and types of perturbations that can be executed simultaneously. Cas12a exhibits a robust ability to carry out multiplex genome editing, whereas Cas9 lacks such capability.

Improved compositions and methods of using Cas12a for genetic engineering are needed in the art.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an overview of zCRISPR-Cas12a and its in vitro characterization. a, Schematic of zCRISPR-Cas12a with enhanced binding affinity between crRNA and target DNA by the extra hydrogen bond (highlighted in blue) offered by Z:T pairing. b, In vitro transcription (IVT) yield of crRNA, quantified by NanoDrop 2000/2000c Spectrophotometer. n=20. c, The heatmap of Ta value between crRNAs and their complementary ssDNAs measured by QuantStudio™ 3 Real-Time PCR System. n=3. d, Normalized in vitro cleavage efficiency of A-crRNA and Z-crRNA mediated AsCas12a-based cleavage on linearized plasmid. The agarose gel electrophoresis bands were quantified by GelAnalyzer 19.1 software. e, In vitro cleavage activity kinetics analysis. AsCas12a RNPs (both A-crRNA and Z-crRNA) were titrated into dsDNA substrates (10 nM) containing target site with TTTT PAM used in FIG. 1d. Cleavage reactions were sampled at distinct time points (10 s, 20 s, 60 s, 100 s, and 600 s), and substrate cleavage was assessed through capillary electrophoresis. Data was fitted using a one-phase decay model, and the corresponding k values are indicated below each figure. n=3. f, Measurement of AsCas12a RNP and dsDNA substrate interactions by Microscale Thermophoresis (MST) assays. The representative target site with six base Z substitutions in crRNA was utilized to quantify binding affinity between AsCas12a RNPs and associated dsDNA substrates. K_{d} values representing the dissociation constants are provided below each figure. Error bars, s.e.m.; n=3.

FIG. 2 shows in cellulo characterization of zCRISPR-Cas12a. a, Overall workflow of zCRISPR-Cas12a in cellulo investigation. b, Genome editing performance affected by the number of Z substitution in the spacer region. The crRNAs with the incremental number of A or Z base (from 0 to 12) in the spacer region were used for genome editing. c, Editing efficiency of A-crRNA and Z-crRNA mediated AsCas12a at two different endogenous sites with crRNA bearing variable length 3′ end truncations or extensions. d, Characterization of the effect of Z substitution in PAM proximal region with seven endogenous sites (left), and the impact shown by the subtracted average indel frequency (the average indel frequency of Z-crRNA minus the average indel frequency of A-crRNA, right). e, Characterization of the effect of Z substitution in PAM distal region with seven endogenous sites (left), and the impact shown by the subtracted average indel frequency (right). f, Validation of zCRISPR-Cas12a on reported low-editing-efficiency sites. 24 previously characterized low-editing-efficiency sites²⁴ with all types of PAMs were selected to verify the efficacy of zCRISPR-Cas12a in HCT116 cells. A, C, G, and T represent four kinds of PAM sequences: TTTA, TTTC, TTTG, and TTTT. g, Performance of coupling Z-crRNA and engineered AsCas12a variant. AsCas12a Ultra was used to further improve the on-target efficiency of ten inefficient enhanced sites in f. h, Relationship between the amount of RNPs and the editing efficiency. Different amounts of RNPs were transfected into HCT116 cells. The indel frequency was measured by next-generation sequencing (NGS) after 72 h cell culture. Error bars, s.e.m.; n=3; nt, nucleotide. Statistical analysis was performed using one-tailed Welch's t-tests, ns=p>0.05; *=p≤0.05; **=p≤0.01; ***=p≤0.001; ****=p≤0.0001.

FIG. 3A-3G shows assessment of off-target effect of CRISPR-Cas12a and zCRISPR-Cas12a and side-by-side comparison with CRISPR-Cas9. FIG. 3A, Matched target sites for AsCas12a and SpCas9 that share a common protospacer sequence. FIG. 3B, Summary of the total number of off-target sites detected by GUIDE-Seq in U2OS cells with six previously characterized sites¹². FIG. 3C, On-target indel frequency measured by NGS. Five low-editing-efficiency matched sites (MSs) were selected from previous study¹² to compare the on-target editing efficiency of CRISPR-Cas12a, zCRISPR-Cas12a, and SpCas9. FIG. 3D, Characteristics of off-target numbers and frequencies determined by GUIDE-Seq in U2OS cells with above five matched sites. Bar graph above represents the total numbers of off-target sites. Heatmap shows the frequencies of potential off-target sites (predicted by Cas-OFFinder, 20 off-target sites, OT1-20) detected by GUIDE-Seq and on-target sites. FIG. 3E, Validation of zCRISPR-Cas12a performance in a complex cell type. Primary human Mesenchymal Stem Cells (hMSCs) were employed to validate the effectiveness, utilizing three sites matched with those in panel FIG. 3C. FIG. 3F, Cell viability assays. Cell viability assessments were conducted to evaluate the health of the four types of cells utilized in this study. The measurements were performed using the CellTiter cell viability assay. FIG. 3G, Evaluation of the Z-crRNA strategy across eight Cas12a orthologs. Site 15 utilized in panel FIG. 3C was chosen to investigate the general applicability of Z-crRNA on FnCas12a, TsCas12a, Mb2Cas12a, Mb3Cas12a, BsCas12a, HkCas12a, PxCas12a, ErCas12a, and LbCas12. The aforementioned purified Cas12a proteins were incubated with A/Z-crRNA to form RNP followed by nucleofection respectively. Error bars, s.e.m.; n=3. Statistical analysis was performed using one-tailed Welch's t-tests, ns=p>0.05; *=p<0.05; **=p≤0.01; ***=p≤0.001; ****=p≤0.0001.

FIG. 4 shows applications of zCRISPR-Cas12a for HDR-mediated knock-in (KI). a, Workflow for HDR-mediated knock-in (KI) experiment. HEK293T cells were transfected with CRISPR-Cas12a and zCRISPR-Cas12a RNPs along with either dsDNA (comprising IRES and EGFP) or ssODN (containing an EcoRI site) donor molecules. The rate of integration was assessed through flow cytometry or NGS analysis, carried out 4 days post nucleofection. b, Analysis of indel frequency for HDR-mediated knock-in assays. On-target efficiency assessment was conducted using the same RNPs employed in the knock-in experiments. c, Gene KI rate reporter system. A double-strand break was created in between the last exon of each housekeeping gene and its 3′ untranslated region (UTR). Gene KI was mediated by a linear dsDNA donor template that contains internal ribosomal entry site (IRES), EGFP sequence and homologous arms (HAs). Blue pentagons represent end modifications of dsDNA donors. d, Structure of the donor end modification. The donor amplification primers were modified with 5′C6-Biotin and 5× phosphorothioate bond. e, KI rate in HEK293T cells using modified dsDNA donors. CRISPR-Cas12a and zCRISPR-Cas12a RNPs were transfected into HEK293T cells coupled with modified donors respectively. Data were collected 4 days post nucleofection by flow cytometry. f, ssODN-mediated KI system. A schematic of the single-stranded oligo DNA (ssODN) mediated knock-in strategy is presented. A double-strand break was induced between the last exon of each housekeeping gene and its 3′ untranslated region (UTR). Knock-in was facilitated by a ssODN donor template containing an EcoRI restriction site flanked by two homologous arms. g, Integration rate measurement in ssODN-mediated KI experiment. The integration rate was evaluated through NGS analysis, with precise EcoRI site knock-in reads as a percentage of the total count. Error bars, s.e.m.; n=3. Statistical analysis was performed using one-tailed Welch's t-tests, ns=p>0.05; *=p≤0.05; **=p≤0.01; ***=p≤0.001; ****=p≤0.0001.

FIG. 5 shows applications of zCRISPR-Cas12a for multiplex genome editing. Analysis of the multiplex genome disruption efficiency in U2OS and HEK293T cells. Five matched sites (MSs) used in previous experiments (FIG. 3c) and other three matched sites⁵⁷, COL8A1 (MS-C), FGF18 (MS-F), and P2RX5-TAX1BP3 (MS-P), were selected for multiplex genome editing assessed by NGS. Double, quadruple, sextuple, or octuple A-crRNAs, Z-crRNAs, or sgRNAs were used to form the corresponding RNPs followed by nucleofection into cells simultaneously. 2×: double-gene knockout; 4×: quadruple-gene knockout; 6×: sextuple-gene knockout; 8×: octuple-gene. Error bars, s.e.m.; n=3.

FIG. 6 shows A/Z-crRNA in vitro synthesis. a, Schematic of crRNA in vitro transcription. SP6 promoter sequence containing dsDNA was served as the template for in vitro transcription. SP6 RNA polymerase (HiScribe™ SP6 RNA Synthesis Kit, NEB-E2070S) was used to synthesize the crRNA. Using ATP in the reaction creates A containing crRNA. ZTP (TriLink Biotechnologies, N-1001, 2-Amino-ATP) was used to replace the ATP in the reaction to generate Z containing crRNA. crRNA yield was measured by NanoDrop 2000/2000c. Created with BioRender.com. b, Agarose gel electrophoresis of synthesized crRNA. 500 ng of each A or Z containing crRNA was loaded in 3% agarose gel for separation.

FIG. 7A-B shows annealing temperature (Ta) measurement. 7A1-A2, Detailed sequence information of selected crRNAs with increasing number of A or Z base. crRNAs and their complementary ssDNAs were listed. The Z base substitution(s) was highlighted. The raw annealing curve was displayed below each corresponding sequence, where the red line represents the annealing temperature curve of A-crRNA/ssDNA and the blue line represents the annealing temperature curve of Z-crRNA/ssDNA. n=3. 7B, Annealing temperature (Ta) measurement program. crRNA and its complementary ssDNA were mixed in the dye containing buffer. Reaction was heated up at 95° C. for 2 min to denature nucleic acids. The reaction was cooled down to 10° C. with the ramp of 0.05° C./s to form the crRNA and ssDNA binary complex. The fluorescence was measured every 0.05 s during the cooling step. NT, nucleotide.

FIG. 8A-1 to 8B-4 shows kinetics Analysis of in vitro cleavage activity and AsCas12a RNP-DNA interaction. 8A-1 to 8A-2 In vitro cleavage activity kinetics analysis. AsCas12a RNPs (both A-crRNA and Z-crRNA) were titrated into dsDNA substrates (10 nM) containing target sites used in FIG. 1d, spanning various PAM sequences. Cleavage reactions were sampled at distinct time points (10 s, 20 s, 60 s, 100 s, and 600 s), and substrate cleavage was assessed through capillary electrophoresis. Data was fitted using a one-phase decay model, and the corresponding k values are indicated below each figure. n=3. 8B-1 to 8B-4 Measurement of AsCas12a RNP and dsDNA substrate interactions by Microscale Thermophoresis (MST) assays. Eight target sites with increasing substitution levels (1 nt substitution to 8 nts substitutions) were utilized to quantify binding affinity between AsCas12a RNPs and associated dsDNA substrates. K_{d} values representing the dissociation constants are provided below each figure. Error bars, s.e.m.; n=3.

FIG. 9A-9E shows Cas12a in vitro cleavage assays. 9A Cleavage on initial tested four sites. Linearized plasmid (Table 3) was digested by A-crRNA and Z-crRNA mediated AsCas12a nuclease. 1% agarose gel was used to separate the cleavage products. Site 1 was TTTA PAM; Site 2 was with the TTTC PAM; Site 3 was with the TTTG PAM; Site 4 was with the TTTT PAM. 9B Cas12a mediated in vitro cleavage on the sites with TTTT PAM. Other nine sites, selected from the plasmid (Table 3), were digested by A-crRNA and Z-crRNA mediated AsCas12a nuclease to verify the performance of zCRISPR-Cas12a on improving the in vitro cleavage efficiency. All nine sites were with the TTTT PAM at the 5′ of the protospacer region. Site 1, 2, 4, 7, and 9 showed the improved cleavage efficiency by using Z-crRNA. [[c,]] 9C AsCas12a mediated in vitro cleavage on the same site with different PAM sequences. The original TTTT PAM for the site 7 in 9B was mutagenized to TTTA, TTTG, and TTTG PAMs on the plasmid respectively. A-crRNA and Z-crRNA were applied to mediate the AsCas12a-based cleavage on the linearized plasmid. 9D Cas12a mediated in vitro cleavage on the fragment amplified from an endogenous gene. ˜3.3 kb DNMT1 gene fragment was amplified from HEK 293T cell genomic DNA. Ten target sites with TTTT PAM were selected for performing the in vitro cleavage assay by AsCas12a. Site 2, 3, 5, 8, and 10 showed the improved cleavage efficiency by using Z-crRNA. 9E In vitro cleavage on linearized plasmid by two Cas12a isoforms. Ten target sites with TTTV PAM were chosen to verify the applicability of zCRISPR-Cas12a using different Cas12a isoforms (left: AsCas12a, right: LbCas12a).

FIG. 10 shows in cellulo genome editing efficiency improved by using Z-crRNA. a, Eight target sites with TTTN PAM were selected for assessing the performance of zCRISPR-Cas12a in GAPDH and HPRT1 genes editing efficiency. Editing efficiency was estimated by T7EI assay using fragment analyzer. b, Genome editing performance affected by the number of Z substitution in the spacer region. The crRNAs with the incremental number of A or Z base (from 0 to 12) in the spacer region were used for RNF2 and TPCN2 genes editing. c, Validation of zCRISPR-Cas12a on reported low-editing-efficiency sites. 24 previously characterized low-editing-efficiency sites with all types of PAMs were selected to verify the efficacy of zCRISPR-Cas12a in HEK293T cells. A, C, G, and T represent four kinds of PAM sequences: TTTA, TTTC, TTTG, and TTTT. d, Performance of coupling Z-crRNA and engineered AsCas12a variant. AsCas12a Ultra was used to further improve the on-target efficiency of ten inefficient enhanced sites in c. e, Genome editing capabilities of Cas9 using both A-crRNA/A-tracrRNA (A-gRNA) guide RNA and Z-crRNA/A-tracrRNA (Z-gRNA) chimeric guide RNA. The validation of genome editing was conducted on the EMX2, FANCF3, and RUNX1 genes in HEK293T cells. n=3. Statistical analysis was performed using one-tailed Welch's t-tests, ns=p>0.05; *=p<0.05; **=p≤0.01; ***=p≤0.001; ****=p≤0.0001.

FIG. 11 shows detailed crRNA sequence information of the selected targeting sites for investigating the essential part of substitution. The seven targeting sites were selected to have the substitutions in the PAM proximal region, and the other seven sites were with the substitution in the PAM distal region. The substituted nucleotides were highlighted.

FIG. 12A-12C shows investigation of cleavage position pattern and indel profile using Z-crRNA. 12A, Exploration of cleavage position pattern. The workflow employed to study the cleavage position pattern is presented. Initially, dsDNA (1 kb) was subjected to digestion by CRISPR-Cas12a and zCRISPR-Cas12a, respectively. The resultant cleavage fragments underwent gel purification, followed by sticky end extension through DNA polymerase. Subsequently, the end-repaired fragments were separately subcloned into a plasmid. Sanger sequencing was conducted to analyze the cleavage position patterns. Created with BioRender.com. 12B, Analysis of indel profiles. Indel profile assessment is described. Genomic DNA edited by CRISPR-Cas12a and zCRISPR-Cas12a at target sites was subjected to Next-Generation Sequencing (NGS) analysis. Visualization of the indel profiles was facilitated through the utilization of the OutKnocker indel analyzing tool.

FIGS. 13A1-13A2 and 13B1-13B2 shows off-target effect of the CRISPR-Cas12a and zCRISPR-Cas12a with six target sites. The off-target effect was determined using GUIDE-Seq in U2OS cells. Mismatched positions in the target sites of off-targets are highlighted in color, and GUIDE-Seq read counts shown to the right of the on- and off-target sequences represent a measure of cleavage efficiency at a given site. The intrinsic poor sequence specific site, Matched Site 6, was chosen as the positive control to ensure our GUIDE-Seq experiments performed successfully.

FIG. 14A-14F shows off-target frequency comparison of CRISPR-Cas12a, zCRISPR-Cas12a and SpCas9 at five matched sites. 14A-14F, The off-target effect was determined using GUIDE-Seq in U2OS cells. Mismatched positions in the target sites of off-targets are highlighted in color, and GUIDE-Seq read counts shown to the right of the on- and off-target sequences represent a measure of cleavage efficiency at a given site. f, Off-target sites predicted by Cas-OFFinder. All matched sites' potential off-target sites were evaluated by Cas-OFFinder software (rgenome.net/cas-offinder/), where we selected up to five mismatches in the spacer region. Homo sapiens (GRCh38/hg38)-Human was chosen as the target genome.

FIG. 15A-15C shows representative flow cytometry analysis of the reporter gene (EGFP) knock-in efficiency. The EGFP⁺ population (P3) in each group was analyzed using a flow cytometry gating strategy. Donor only groups were included as a control to show the background fluorescence from the donor. In these experiments, all crRNAs were with 18 nt spacer (n=3).

FIG. 16A-16C shows representative flow cytometry analysis of the reporter gene (EGFP) knock-in efficiency and gating strategy. 16A EGFP donor knock-in efficiency measured by flow cytometry (n=3). 16B1-16B2 The EGFP⁺ population (P3) in each group was analyzed using a flow cytometry gating strategy. In these experiments, all crRNAs were with 20 nt spacer (n=3). 16C Gating strategy. 1) FSC-A/SSC-A was used to remove debris; 2) FSC-A/FSC-W was used to define single cells; 3) FSC-A/FITC-A was used to determine fluorescence.

### SUMMARY

An aspect provides a recombinant clustered regularly interspaced short palindromic repeat (CRISPR) RNA (crRNA) comprising a scaffold region and a spacer region, wherein one or more 2-aminoadenine nucleotides are present in the spacer region of the crRNA. The one or more 2-aminoadenine nucleotides can be substituted for one or more adenosine nucleotides. The crRNA can comprise 2, 3, 4, 5, 6, 7, or more 2-aminoadenine nucleotides in the spacer region of the crRNA. The crRNA can comprise 7 or more 2-aminoadenine nucleotides in the spacer region of the crRNA. The crRNA can be present in a lipid nanoparticle or lipoplex.

Another aspect provides a recombinant clustered regularly interspaced short palindromic repeat (CRISPR)-associated (Cas) system comprising (i) one or more crRNAs each comprising a scaffold region and a spacer region capable of hybridizing to a target nucleic acid molecule, wherein the spacer region comprises one or more 2-aminoadenine nucleotides and (ii) a Cas12a protein or a nucleic acid molecule encoding the Cas12a protein. The one or more crRNAs, the Cas12a protein, the nucleic acid molecule encoding the Cas12a protein, or combinations thereof can be present in a lipid nanoparticle or lipoplex. The Cas12a protein can be Acidaminococcus sp., Cas12a, Francisella novicida U112 (FnCas12a), Thiomicrospira sp. XS5 (TsCas12a), Moraxella bovoculi AAX08_00205 (Mb2Cas12a), Moraxella bovoculi AAX11_00205 (Mb3Cas12a), Butyrivibrio sp. NC3005 (BsCas12a), Helcococcus kunzii ATCC 51366 (HkCas12a), Pseudobutyrivibrio xylanivorans DSM 10317 (PxCas12a), Eubacterium rectale (ErCas12a), or Lachnospiraceae bacterium ND2006 (LbCas12a), a dCas12a (endonuclease activity deactivated mutant), a Cas12a-based base editor, a Cas12a-based prime editor, or a Cas12a-based CRISPRa/i system can also be used. The recombinant CRISPR-Cas system can further comprise one or more single-stranded donor nucleic acid molecules and/or double-stranded donor nucleic acid molecules.

Yet another aspect provides a method of modifying a cell comprising delivering to the cell (i) a Cas12a protein or a nucleic acid molecule encoding a Cas12a protein and (ii) one or more crRNAs comprising a scaffold region and one or more spacer regions capable of specifically hybridizing to one or more target nucleic acid molecules, wherein the one or more spacer regions comprise one or more 2-aminoadenine nucleotides. The one or more crRNAs, the Cas12a protein, the nucleic acid molecule encoding the Cas12a protein, or combinations thereof can be present in a lipid nanoparticle or lipoplex. The method can be an in vitro method or an in vivo method. The cell can be a eukaryotic cell or a prokaryotic cell. The method can further comprise delivering to the cell a donor nucleic acid molecule. The donor nucleic acid molecule can be a single-stranded donor nucleic acid molecule or a double-stranded donor nucleic acid molecule. The one or more crRNAs can comprise 2, 3, 4, 5, 6, 7, or more 2-aminoadenine nucleotides in the spacer region. The one or more crRNAs can comprise 7 or more 2-aminoadenine nucleotides in the spacer region. The method can use 2, 3, 4, 5, 6, 7, 8, or more different crRNAs capable of specifically hybridizing to 2, 3, 4, 5, 6, 7, 8, or more target nucleic acid molecules, which are delivered to the cell, wherein each of the different crRNAs comprise one or more 2-aminoadenine nucleotides in the spacer region. The method can have an editing efficiency that is improved as compared to a method wherein the one or more crRNAs do not comprise one or more 2-aminoadenine nucleotides. The method can have an off-target cleavage frequency that is not increased as compared to a method wherein the one or more crRNAs do not comprise one or more 2-aminoadenine nucleotides. The concentration of the Cas12a protein and the one or more crRNAs can be reduced as compared to a method wherein the one or more crRNAs do not comprise one or more 2-aminoadenine nucleotides. The one or more crRNAs can comprise two or more spacer regions, wherein the Cas12a protein can create a deletion of the one or more target nucleic acid molecules. A direct repeat sequence can separate each spacer in the crRNA. The nucleic acid molecule encoding the Cas12a protein can be present in a viral expression vector, a plasmid expression vector, or a linear/circular mRNA molecule (see, e.g., Liang et al., Nat Biotechnol (2024). doi.org/10.1038/s41587-023-02095-x). The Cas12a protein and the one or more crRNAs can be allowed to associate prior to delivery to the cell.

Another aspect provides a method for site-specific integration of a donor nucleic acid molecule into a target nucleic acid molecule. The method comprises introducing into a cell: (i) a Cas12a protein or a nucleic acid molecule encoding a Cas12a protein; (ii) one or more crRNA molecules comprising a scaffold region and one or more spacer regions capable of hybridizing to the target nucleic acid molecule, wherein the one or more spacer regions comprise one or more 2-aminoadenine nucleotides, and (iii) a donor nucleic acid molecule; wherein the donor nucleic acid is inserted into the target nucleic acid molecule at a site-specific target site. The donor nucleic acid molecule can include homology arms flanking a sequence for integration into the target site. The one or more crRNAs, the Cas12a protein, the nucleic acid molecule encoding the Cas12a protein, the donor nucleic acid molecule, or combinations thereof can be present in a lipid nanoparticle or lipoplex. The method can be an in vitro method or an in vivo method. The cell can be a eukaryotic cell or a prokaryotic cell. The donor nucleic acid molecule can be a single-stranded donor nucleic acid molecule or a double-stranded donor nucleic acid molecule. The one or more crRNAs can comprise 2, 3, 4, 5, 6, 7, or more 2-aminoadenine nucleotides in the spacer region of the one or more crRNAs. The one or more crRNAs can comprise 7 or more 2-aminoadenine nucleotides in the spacer region of the one or more crRNAs. The method can use 2, 3, 4, 5, 6, 7, 8, or more different crRNAs capable of specifically hybridizing to 2, 3, 4, 5, 6, 7, 8, or more target nucleic acid molecules, which are delivered to the cell, wherein each of the different crRNAs comprise one or more 2-aminoadenine nucleotides in the spacer region. The editing efficiency of the method can be improved as compared to a method wherein the one or more crRNAs do not comprise one or more 2-aminoadenine nucleotides. In an aspect, the off-target cleavage frequency is not increased as compared to a method wherein the one or more crRNAs do not comprise one or more 2-aminoadenine nucleotides. A concentration of the Cas12a protein and the one or more crRNAs can be reduced as compared to a method wherein the crRNA does not comprise one or more 2-aminoadenine nucleotides without compromising the editing efficiency. The nucleic acid molecule encoding the Cas12a protein can be present in a viral expression vector or a plasmid expression vector, or a linear/circular mRNA molecule. The Cas12a protein and the one or more crRNAs can be allowed to associate prior to delivery to the cell.

An aspect provides a method of reducing expression of a target gene in a cell. The can comprise introducing into the cell (a) one or more crRNA molecules comprising a scaffold region and one or more spacer regions capable of hybridizing to the target gene, wherein the one or more spacer regions comprise one or more 2-aminoadenine nucleotides and (b) a Cas12a protein or a nucleic acid molecule encoding a Cas12a protein, wherein the one or more crRNA molecules hybridize to the target gene and the Cas12a cleaves the target gene, such that the target gene expression is reduced in the cell relative to a cell in which the one or more crRNAs and the Cas12a protein or nucleic acid molecule encoding a Cas12a protein are not introduced. The one or more crRNAs, the Cas12a protein, the nucleic acid molecule encoding the Cas12a protein, or combinations thereof can be present in a lipid nanoparticle or lipoplex. The method can be an in vitro method or an in vivo method. The cell can be a eukaryotic cell or a prokaryotic cell. The one or more crRNAs can comprise 2, 3, 4, 5, 6, 7, or more 2-aminoadenine nucleotides in the spacer region of the one or more crRNAs. The one or more crRNAs can comprise 7 or more 2-aminoadenine nucleotides in the spacer region of the one or more crRNAs. The method can use 2, 3, 4, 5, 6, 7, 8, or more different crRNAs capable of specifically hybridizing to 2, 3, 4, 5, 6, 7, 8, or more target nucleic acid molecules, which are delivered to the cell, wherein each of the different crRNAs comprise one or more 2-aminoadenine nucleotides in the spacer region. The editing efficiency of the method can be improved as compared to a method wherein the one or more crRNAs do not comprise one or more 2-aminoadenine nucleotides. In some aspects, the off-target cleavage frequency of the method is not increased as compared to a method wherein the one or more crRNAs do not comprise one or more 2-aminoadenine nucleotides. A concentration of the Cas12a protein and the one or more crRNAs can be reduced as compared to a method wherein the one or more crRNA molecules do not comprise one or more 2-aminoadenine nucleotides without compromising the high-level editing efficiency. The crRNA can comprise two or more spacer regions, and the Cas12a protein can create a deletion of the one or more target nucleic acid molecules. A direct repeat sequence can separate each spacer in the crRNA. The nucleic acid molecule encoding the Cas12a protein can be present in a viral expression vector or a plasmid expression vector, or a linear/circular mRNA molecule. The Cas12a protein and the one or more crRNAs can be allowed to associate prior to delivery to the cell.

Yet another aspect provides a pharmaceutical composition comprising a complex comprising (i) one or more crRNA molecules comprising a scaffold region and one or more spacer regions capable of hybridizing to a target gene, wherein the one or more spacer regions comprise one or more 2-aminoadenine nucleotides; (ii) a Cas12a protein; and a pharmaceutical carrier or excipient. The one or more crRNAs, the Cas12a protein, or combinations thereof can be present in a lipid nanoparticle or lipoplex.

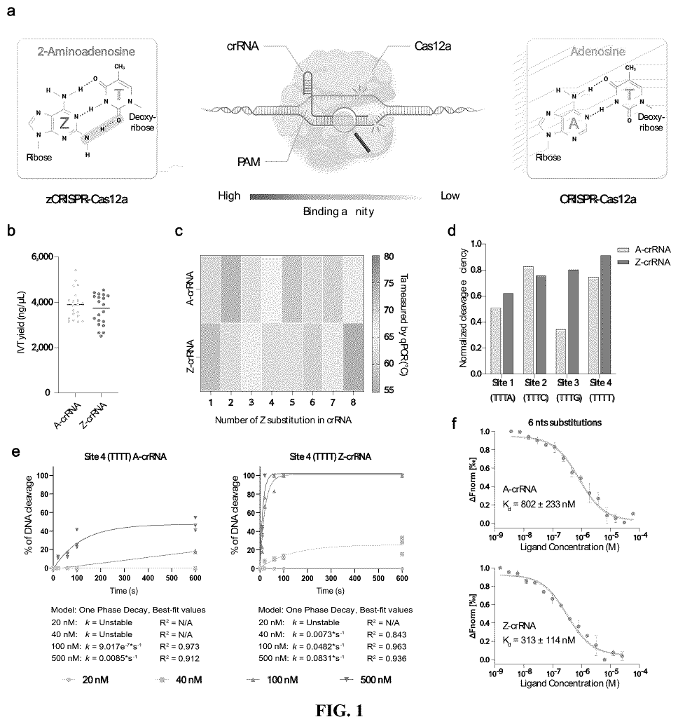

Therefore, provided herein are novel CRISPR-Cas12a crRNA compositions and engineering strategies based on incorporating 2-aminoadenine (Z) into crRNA (Z-crRNA) to improve the binding affinity and target recognition, which results in a genome editing tool superior to the most widely used CRISPR-Cas9 system (FIG. 1a). Base Z alters the Watson-Crick two hydrogen bonds pairing between adenine and thymine (A:T) to three hydrogen bonds pairing (Z:T), which can increase thermal stability, sequence specificity, and nuclease resistance. The working examples demonstrate that the substitution of A with Z in crRNA dramatically improves the on-target editing efficiency of Cas12a while maintaining its intrinsic low off-target effect in mammalian cells.

### DETAILED DESCRIPTION

There is a growing interest of using Cas12a for clinical genome editing because of its intrinsic high fidelity. Moreover, unlike the ˜100-mer sgRNA of Cas9, its short 40-43-mer crRNA can be readily chemically manufactured. However, the application of the CRISPR-Cas12a system for human genome editing is still limited by its relatively low editing efficiency. In recent years, various approaches have been developed to improve the Cas12a-based genome editing efficiency in mammalian cells, which are based on either protein engineering or RNA engineering. Employing chemically modified guide RNA represents a key approach in RNA engineering. Previous studies are predominantly concentrated on chemical modifications of its sugar group and backbone, whereas investigations pertaining to base modification of guide RNA remain rare. To the best of our knowledge, there is no precedent in literature where the genome editing efficiency of Cas12a is enhanced by increasing the interaction between guide RNA and its target complementary DNA strand through base modification.

### Polynucleotides

Polynucleotides contain less than an entire genome and can be single- or double-stranded nucleic acid molecules. A polynucleotide can be RNA, DNA, cDNA, genomic DNA, chemically synthesized RNA or DNA or combinations thereof. A polynucleotide can comprise, for example, a gene, open reading frame, non-coding region, crRNA, or regulatory element.

A gene is any polynucleotide molecule that encodes a polypeptide, protein, or fragment thereof, optionally including one or more regulatory elements preceding (5′ non-coding sequences) and following (3′ non-coding sequences) the coding sequence. In one aspect, a gene does not include regulatory elements preceding and following the coding sequence. A native or wild-type gene refers to a gene as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources, or regulatory elements and coding sequences that are derived from the same source but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide.

Polynucleotides can comprise additional heterologous nucleotides that do not naturally occur contiguously with the polynucleotides. As used herein the term “heterologous” refers to a combination of elements that are not naturally occurring or that are obtained from different sources.

Polynucleotides can be isolated. An isolated polynucleotide is a naturally-occurring polynucleotide that is not immediately contiguous with one or both of the 5′ and 3′flanking genomic sequences that it is naturally associated with. An isolated polynucleotide can be, for example, a recombinant DNA molecule of any length, provided that the nucleic acid sequences naturally found immediately flanking the recombinant DNA molecule in a naturally-occurring genome is removed or absent. Isolated polynucleotides also include non-naturally occurring nucleic acid molecules. Polynucleotides can encode full-length polypeptides, polypeptide fragments, and variant or fusion polypeptides.

Degenerate polynucleotide sequences encoding polypeptides described herein, as well as homologous nucleotide sequences that are at least about 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a polypeptide described herein or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also are polynucleotides.

Polynucleotides can be obtained from nucleic acid molecules present in, for example, a mammal, plant, yeast, or bacterium. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides can comprise non-coding sequences or coding sequences for naturally occurring polypeptides or can encode altered sequences that do not occur in nature. Unless otherwise indicated, the term polynucleotide or gene includes reference to the specified sequence as well as the complementary sequence thereof.

The expression products of genes or polynucleotides are often proteins, or polypeptides, but in non-protein coding genes such as crRNA genes, rRNA genes, or tRNA genes, the product is a functional RNA. The process of gene expression is used by all known life forms, i.e., eukaryotes (including multicellular organisms), prokaryotes (bacteria and archaea), and viruses, to generate the macromolecular machinery for life. Several steps in the gene expression process can be modulated, including the transcription, up-regulation, RNA splicing, translation, and post-translational modification of a protein.

A polynucleotide can be a cDNA sequence or a genomic sequence. A genomic sequence is a sequence that is present or that can be found in the genome of an organism or a sequence that has been isolated from the genome of an organism. A cDNA polynucleotide can include one or more of the introns of a genomic sequence from which the cDNA sequence is derived. As another example, a cDNA sequence can include all of the introns of the genomic sequence from which the cDNA sequence is derived. Complete or partial intron sequences can be included in a cDNA sequence.

Polynucleotides as set forth in SEQ ID NO:1 through SEQ ID NO:383, a functional fragment thereof; or having at least 95% identity to SEQ ID NO:1-SEQ ID NO:383, are provided herein. In some aspects, the isolated polynucleotides have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, and any number or range in between, identity to SEQ ID NO:1 through SEQ ID NO:383 or a functional fragment thereof.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some aspects the length of a reference sequence aligned for comparison purposes is at least 80% of the length of the comparison sequence, and in some aspects is at least 90% or 100%. In an aspect, the two sequences are the same length.

Ranges of desired degrees of sequence identity are approximately 80% to 100% and integer values in between. Percent identities between a disclosed sequence and a claimed sequence can be at least 80%, at least 83%, at least 85%, at least 90%, at least 95%, at least 98%, at least 99%, at least 99.5%, or at least 99.9%. In general, an exact match indicates 100% identity over the length of the reference sequence.

Polypeptides and polynucleotides that are sufficiently similar to polypeptides and polynucleotides described herein can be used herein. Polypeptides and polynucleotides that are about 90, 91, 92, 93, 94 95, 96, 97, 98, 99 99.5% or more identical to polypeptides and polynucleotides described herein can also be used herein. For example, a polynucleotide can have 80% 85%, 90%, 91%, 92%, 93%, 94%, 95%, 96%, 97%, 98%, 99% or more identity to any of the SEQ ID NOs described herein.

### Polypeptides

A polypeptide is a polymer where amide bonds covalently link three or more amino acids. A polypeptide can be post-translationally modified. A purified polypeptide is a polypeptide preparation that is substantially free of cellular material, other types of poly peptides, chemical precursors, chemicals used in synthesis of the polypeptide, or combinations thereof. A polypeptide preparation that is substantially free of cellular material, culture medium, chemical precursors, chemicals used in synthesis of the polypeptide has less than about 30%, 20%, 10%, 5%, 1% or less of other polypeptides, culture medium, chemical precursors, and/or other chemicals used in synthesis. Therefore, a purified polypeptide is about 70%, 80%, 90%, 95%, 99% or more pure.

The term “polypeptides” can refer to one or more types of polypeptides or a set of polypeptides. “Polypeptides” can also refer to mixtures of two or more different types of polypeptides including, but not limited to, full-length proteins, truncated polypeptides, or polypeptide fragments. The term “polypeptides” or “polypeptide” can each mean “one or more polypeptides.”

### Vectors and Delivery Vehicles

Nucleic acid molecules described herein can be contained within a vector that is capable of directing their expression in, for example, a cell that has been transduced with the vector. Vectors can be introduced and propagated in a prokaryotic cell or a eukaryotic cell. Vectors can be useful for autonomous replication in a host cell or can be integrated into the genome of a host cell upon introduction into the host cell, and thereby are replicated along with the host genome (e.g., non-episomal mammalian vectors).

In some aspects, a vector is an expression vector. Expression vectors can direct the expression of sequences to which they are operably linked. In some aspects, a vector is a eukaryotic expression vector, i.e. the vector can direct the expression of coding sequences to which they are operably linked in a host cell. In general, expression vectors can be plasmid vectors, viral vectors (e.g., replication defective retroviruses, adenoviruses, and adeno-associated viruses), or other suitable vectors.

Vectors can be introduced into host cells via transformation or transfection techniques. Suitable methods for transforming or transfecting host cells can be found in Sambrook et al. (1989) Molecular Cloning: A Laboratory Manual (2nd ed., Cold Spring Harbor Laboratory Press, Plainview, N.Y.) and other standard molecular biology laboratory manuals.

A viral vector can be a nucleic acid molecule that includes virus-derived nucleic acid elements that typically facilitate transfer of the nucleic acid molecule or integration into the genome of a cell. A viral vector can also be a viral particle that mediates nucleic acid transfer. Viral particles typically include viral components, and sometimes also host cell components, in addition to nucleic acid(s). Retroviral vectors can contain structural and functional genetic elements, or portions thereof, that are primarily derived from a retrovirus. Retroviral lentivirus vectors contain structural and functional genetic elements, or portions thereof including LTRs, that are primarily derived from a lentivirus (a sub-type of retrovirus).

In some aspects, nucleic acids molecules can be delivered by delivery vehicles. For example, a nucleic acid molecule can be stably integrated into the host genome, can be episomally replicating, or can be present in the recombinant host cell as a mini-circle expression vector for stable or transient expression. Therefore, a nucleic acid molecule can be maintained and replicated in a recombinant host cell as an episomal unit. In some aspects, a nucleic acid molecule can be stably integrated into the genome of the recombinant cell. Stable integration can also be accomplished using classical random genomic recombination techniques or with more precise genome editing techniques such as using guide RNA-directed CRISPR/Cas9, DNA-guided endonuclease genome editing or TALENs genome editing (transcription activator-like effector nucleases). In an aspect, a nucleic acid molecule can present in the recombinant host cell as a mini-circle expression vector for stable or transient expression.

Nucleic acid molecules can be encapsulated in a viral capsid (e.g., adeno-associated virus (AAV)) or a lipid nanoparticle (or other suitable delivery vehicle) and transduced into cells.

Delivery of Editing Effectors by Lipid Nanoparticle (LNP) Encapsulated mRNA

Any of the nucleic acid molecules, crRNA molecules, vectors, and/or pharmaceutical formulations can be formulated in or administered via a lipid nanoparticle or lipoplex. See e.g., WO20171173054, which is hereby incorporated by reference in its entirety. Lipid nanoparticles can be about 100 nm or less in size. LNPs can be formed by mixing a lipid component (e.g., in ethanol) with an aqueous nucleic acid molecule component. Lipoplexes are particles formed by bulk mixing lipids and nucleic acid molecule components. Lipoplexes can be about 100 nm and 1 micron in size. In certain embodiments the lipid nucleic acid assemblies are LNPs. A lipid nucleic acid molecule assembly can comprise one or more types of lipid molecules physically associated with each other by intermolecular forces. A lipid nucleic acid molecule assembly can comprise a bioavailable lipid having a pKa value of about less than about 7.5. Lipid nucleic acid assemblies can be formed by mixing an aqueous nucleic acid-containing solution with an organic solvent-based lipid solution, e.g, 100% ethanol. Suitable solutions or solvents can contain in some examples water, PBS, Tris buffer, NaCl citrate buffer, ethanol, chloroform, diethylether, cyclohexane, tetrahydrofuran, methanol, and/or isopropanol. A pharmaceutically acceptable buffer can optionally be present in a pharmaceutical formulation comprising the lipid nucleic acid molecules assemblies, e.g., for an ex vivo therapy. In some aspects, an aqueous solution can comprise any of the nucleic acid molecules, crRNA molecules, vectors, and/or pharmaceutical formulations described herein. In some embodiments, an aqueous solution can further comprise mRNA molecules encoding Cas12a or variants thereof.

LNPs can include microspheres (including unilamellar and multilamellar vesicles, e.g., “liposomes,” comprising lamellar phase lipid bilayers that can comprise an aqueous core, e.g, comprising a substantial portion of any of the nucleic acid molecules, crRNA molecules, vectors, and/or pharmaceutical formulations described herein), a dispersed phase in an emulsion, micelles, or an internal phase in a suspension. Emulsions, micelles, and suspensions can also be used.

In some aspects, lipid nucleic acid molecule assembly compositions include an amine lipid (e.g., an ionizable lipid or a biodegradable lipid), together with an optional helper lipid, a neutral lipid, and a stealth lipid such as a PEG lipid. In some aspects, the amine lipids or ionizable lipids are cationic depending on the pH.

In some aspects, lipid nucleic acid assembly compositions comprise an amine lipid, which is, for example an ionizable lipid such as Lipid A or its equivalents, including acetal analogs of Lipid A. In some aspects, an amine lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate..

In some aspects, an amine lipid is an analog of Lipid A. In some aspects, a Lipid A analog is an acetal analog of Lipid A, such as a C4-C12 acetal analog, a C5-C12 acetal analog, a C5-C10 acetal analog, or a C4, C5, C6, C7, C9, C10, C11, or C12 acetal analog. Amine lipids and other biodegradable lipids suitable for use in the lipid nucleic acid molecule assemblies described herein can be biodegradable in vivo or ex vivo. Amine lipids can have low toxicity (e.g., are tolerated in animal models without adverse effect in amounts of greater than or equal to 10 mg/kg). In some aspects, lipid nucleic acid molecule assemblies comprising an amine lipid include those where at least 50% of the nucleic acid molecules, e.g., mRNA or crRNA, or lipid nucleic acid molecule assemblies are cleared from the plasma within 8, 10, 12, 24, or 48 hours, or 3, 4, 5, 6, 7, or 10 days. Biodegradable lipids include, for example the biodegradable lipids of WO/2020/219876, WO/2020/118041, WO/2020/072605, WO/2019/067992, WO/2017/173054, WO2015/095340, and WO2014/136086, which are hereby incorporated herein by reference. Lipid clearance can be measured as described in, for example Maier et al. Mol Ther. 2013, 21(8), 1570-78.

Neutral lipids can be used in lipid nucleic acid molecules assembly compositions. Neutral lipids include, for example, a variety of neutral, uncharged or zwitterionic lipids. Examples of neutral phospholipids include, for example, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE).

Helper lipids can be used in lipid nucleic acid molecules assemblies and include steroids, sterols, alkyl resorcinols, cholesterol, 5-heptadecylresorcinol, and cholesterol hemisuccinate. Stealth lipids can be used in lipid nucleic acid molecules assemblies and are lipids that alter the length of time lipid nanoparticles can exist in vivo. Stealth lipids can assist in the formulation process by, for example, reducing particle aggregation and controlling particle size. Stealth lipids can modulate pharmacokinetic properties of the lipid nucleic acid assembly molecules or aid in stability of nanoparticles ex vivo. Stealth lipids include stealth lipids having a hydrophilic head group linked to a lipid moiety as described in, e.g., Romberg et al., Pharmaceutical Research, Vol. 25, No. 1, 2008, pg. 55-71 and Hoekstra et al, Biochimica et Biophysica Acta 1660 (2004) 41-52. Additional suitable PEG lipids are disclosed, e.g., in WO 2006/007712.

In some aspects, a hydrophilic head group of stealth lipid comprises a polymer moiety based on PEG. A stealth lipid can comprise a polymer moiety selected from polymers based on PEG, poly(oxazoline), poly(vinyl alcohol), poly(glycerol), poly(N-vinylpyrrolidone), polyaminoacids, and poly[N-(2-hydroxypropyl)methacrylamide].

A PEG lipid can further comprise a lipid moiety. A lipid moiety can be derived from diacylglycerol or diacylglycamide, including a dialkylglycerol or dialkylglycamide group having alkyl chain length independently comprising from about C4 to about C40 saturated or unsaturated carbon atoms, wherein the chain can comprise one or more functional groups such as, for example, an amide or ester. In some aspects, an alkyl chain length comprises about C10 to C20. A dialkylglycerol or dialkylglycamide group can further comprise one or more substituted alkyl groups.

Other methods and compositions can be used for delivery of nucleic acids and include used of electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

### Cas12a

In an aspect, a Cas12a protein or a nucleic acid encoding a Cas12a protein is provided. One or more different Cas12a proteins can be used in any of the systems, compositions or methods described herein. Cas12a proteins include, for example, AsCas12a, an RNA guided class II nuclease derived from Acidaminococcus sp. (AsCas12a), Francisella novicida U112 (FnCas12a), Thiomicrospira sp. XS5 (TsCas12a), Moraxella bovoculi AAX08_00205 (Mb2Cas12a), Moraxella bovoculi AAX11_00205 (Mb3Cas12a), Butyrivibrio sp. NC3005 (BsCas12a), Helcococcus kunzii ATCC 51366 (HkCas12a), Pseudobutyrivibrio xylanivorans DSM 10317 (PxCas12a), Eubacterium rectale (ErCas12a), and Lachnospiraceae bacterium ND2006 (LbCas12a), Sulfuricurvum sp. PC08-66 (Ss2Cas12a), Pseudobutyrivibrio ruminus Cf1 b (PrCas12a), Oribacterium sp. Nk2B42 (OsCas12a), Arcobacter butzleri L348 (AbCas12a), Flavobacterium branchiophilum FL-15 (FbCas12a), Bacteroidetes oral taxon 274 (BoCas12a), Bacteroidales bacterium KA00251 (BbCas12a), Anaerovibrio sp. RM50 (As2Cas12a), Candidatus Peregrinibacteria GW2011 (PbCas12a), Treponema endosymbiont of Eucomonympha sp. (EsCas12), Parcubacteria group bacterium GW2011 (PgbCas12a), Candidatus Roizmanbacteria bacterium GW2011 (RbCas12a), Uncultured bacterium (gcode 4) ACD 3000058 (U4Cas12a), Sneathia amnii strain SN3 (SaCas12a), Candidatus Methanomethylophilus alvus Mx1202 (MaCas12a), Agathobacter rectalis 2789STDY5834884 (ArCas12a), Lachnospira pectinoschiza strain 2789STDY5834886 (LpCas12a), or any other suitable Cas12a protein. A Cas12a protein can also comprise a dCas12a protein, which can be used to, for example, repress expression of genes. Examples include dAsCas12a), dFnCas12a, dTsCas12a, dMb2Cas12a, dMb3Cas12a, dBsCas12a, dHkCas12a, dPxCas12a, dErCas12a, dLbCas12a, dSs2Cas12a, dPrCas12a, dOsCas12a, dAbCas12a), dFbCas12a, dBoCas12a, dBbCas12a, dAs2Cas12a, PbCas12a, dEsCas12, dPgbCas12a, dRbCas12a, dU4Cas12a, dSaCas12a, dMaCas12a, dArCas12a, dLpCas12a, or any other suitable dCas12a (endonuclease activity deactivated mutant). Cas12a-based base editors (see e.g., Kleinstiver et al., Nat. Biotechnol. 37:276 (2019); Li et al., Nat. Biotechnol. 36:324 (2018); Wang et al., Cell Rep. 31:107723 (2020)), Cas12a-based prime editors (see, e.g. Nat. Biotechnol. (2024). doi.org/10.1038/s41587-023-02095-x), or Cas12a-based CRISPRa/i systems (see, e.g., Bryson et al., Methods Mol Biol. 2024; 2774:193-204; Griffith et al., Cell Genom. 2023 Sep. 1; 3(9):100387; Kieu Nguyen et al., Biomaterials. 2023 June; 297:122106; Ming et al., Hortic Res. 2022 Jun. 30; 9:uhac148; Hsiung et al., Nat Biotechnol. 2024 May 17 doi: 10.1038/s41587-024-02224-0; Joseph et al., Synth Syst Biotechnol. 2022 Dec. 24; 8(1):148-156) can also be used herein. A recombinant CRISPR-Cas system can also be used in the compositions and methods described herein.

In an aspect, a nucleic acid molecule encoding a Cas12a protein can be used in the methods and systems of genetic engineering. Nucleic acid molecules encoding a Cas12a protein can be present in a vector as described above, including e.g., a viral expression vector, a DNA expression vector, a plasmid vector, a bacterial expression vector, a eukaryotic expression vector, or a linear/circular mRNA molecule.

crRNA

In an aspect, a CRISPR Cas12a system recognizes a PAM sequence of TTTA, TTTC, TTTG, and/or TTTT.

A crRNA can comprise a scaffold region and a spacer region comprising a sequence homologous to a target nucleic acid molecule or target gene. A scaffold region can comprise a right stem region, a loop region, and a left loop region.

Examples of scaffold sequences for crRNA molecules for Cas12a proteins are shown below:

(SEQ ID NO: 260)
AUUUCUACUAUUGUAGAU
(SEQ ID NO: 261)
AUUUCUACUGUUGUAGAU
(SEQ ID NO: 262)
AUUUCUACUUUUGUAGAU
(SEQ ID NO: 263)
AUUUCUACUAGUGUAGAU
(SEQ ID NO: 264)
AUUUCUACUGUGUGUAGAU
AUUUCUACUX₁X₂X₃X₄GUAGAU,

wherein X₁ is G, U, orA; wherein X₂ is U or G, wherein X₃ is G or U, and wherein X4 is U that is present or absent (SEQ ID NO:265). The bold nucleotides of SEQ ID NO:xx are the stem left region. The underlined nucleotides are the stem right region. Between the stem left and stem right region is the loop region.

In an aspect, a crRNA comprises 1, 2, 3, 4, 5, 6, 7, 8, 9, 10, 11, 12, 13, 14, 15 or more 2-aminoadenine nucleotides. In an aspect, one or more 2-aminoadenine nucleotides are substituted for one or more adenosine nucleotides in the spacer region. In an aspect, less than 5, 4, 3, or 2 2-aminoadenine nucleotides are present in a scaffold region. In an aspect, no 2-aminoadenine nucleotides are present in a scaffold region.

A spacer region follows the scaffold region. Spacer regions can be designed to be homologous to a target gene or target nucleic acid molecule. Examples of spacer regions are shown in SEQ ID NO:1-131, 266-325, 347-383.

In an aspect, one or more 2-aminoadenine nucleotides are present throughout the spacer region. In an aspect, one or more 2-aminoadenine nucleotides are present in the proximal part (i.e., the 5′ end) of the spacer region (e.g., within the first 10 nucleotides of the spacer region). In an aspect, one or more 2-aminoadenine nucleotides are present in the distal part (i.e., the 3′ end) of the spacer region A distal end of a spacer region can be within about the last 10 nucleotides (i.e., the 3′ end) of the spacer region.

a crRNA can comprise two or more spacer regions (e.g., 2, 3, 4, 5, 6, 7, 8, 9, 10, or more) spacer regions. A direct repeat sequence can separate each spacer in the crRNA.

A spacer region of a crRNA can be configured to hybridize to a target nucleic acid molecule. For example, a spacer region in a crRNA can have sequences that are complementary to a target nucleic acid sequence. The complementarity can be partial complementarity or complete (e.g., perfect) complementarity. The complementarity of two polynucleotide strands is achieved by distinct interactions between nucleobases: adenine (A), thymine (T) (uracil (U) in RNA), guanine (G), and cytosine (C). Adenine and guanine are purines, while thymine, cytosine, and uracil are pyrimidines. Both types of molecules complement each other and can only base pair with the opposing type of nucleobase by hydrogen bonding. For example, an adenine can only be efficiently paired with a thymine (A-T) or a uracil (A-U), and a guanine can only be efficiently paired with a cytosine (G-C). In an aspect, a Z base as described herein pairs with an T (Z-T) and is considered a complementary pair. The base complement A-T or A-U share two hydrogen bonds, while the base pair G-C shares three hydrogen bonds. The two complementary strands are oriented in opposite directions, and they are said to be antiparallel. For another example, the sequence 5′-A-G-T 3′ binds to the complementary sequence 3′-T-C-A-5′. The degree of complementarity between two strands can vary from complete (or perfect) complementarity to no complementarity. The degree of complementarity between polynucleotide strands can affect the efficiency and strength of the hybridization between the nucleic acid strands. In some aspects, the spacer regions of crRNA molecules have perfect complementary to a target nucleic acid molecule over the whole length of the spacer region of the crRNA molecule. Perfectly complementary provides that the spacer region of the crRNA molecule is complementary to the target nucleic acid molecule at 100% of the bases, with no overhangs on either end of either strand. In some aspects, the crRNA and target nucleic acid molecule have 80, 85, 86, 87, 88, 89, 90, 91, 92, 93, 94, 95, 96, 97, 98, 99% or more complementarity.

In some aspects, a Cas12a system comprises one or more crRNAs, and each spacer in at least a portion of the one or more crRNAs is configured to hybridize to the same target nucleic acid. In other aspects, a Cas12a system comprises one or more crRNAs, and each spacer in at least a portion of the one or more crRNAs is configured to hybridize to a different target nucleic acid molecule. For example, 2, 3, 4, 5, 6, 7, 8, 9, 10, 15, 20 or more different crRNAs can be used to target 2, 3, 4, 5, 6, 7, 8, 9, 10, 15, 20 or more different target nucleic acid molecules. In other aspects, a Cas12a system comprises one or more crRNAs, and each spacer in at least a portion of the one or more crRNAs is configured to hybridize to a same target nucleic acid molecule. In certain aspects, a Cas12a system comprises one or more crRNAs, and each spacer in all of the one or more crRNAs is configured to hybridize to a different target nucleic acid molecule.

A target nucleic acid molecule of a Cas12a system is a nucleic acid molecule to which a spacer sequence is designed to have complementarity, where hybridization between a target nucleic acid molecule and a spacer sequence promotes the formation of a CRISPR complex. A target nucleic acid molecule can comprise an endogenous gene. A target nucleic acid molecule can be an RNA molecule or a DNA molecule (e.g., a double-stranded DNA molecule). A target nucleic acid molecule can be derived from genomic DNA, mitochondria DNA, chloroplast DNA, or viral DNA and can be present in a host cell or in an in vitro environment.

In some aspects, a target nucleic acid molecule is a genomic site or DNA locus capable of being recognized by and bound to a crRNA. An enzymatically active crRNA-Cas complex can process such a target site to result in a break at the CRISPR target site. In the case of a deactivated Cas12a (dCas12a), a crRNA-dCas12a still recognizes and binds a CRISPR target site without cutting the target nucleic acid (e.g., the target nucleic acid molecule).

A target nucleic acid molecule can be, for example, a transcription factor, a metabolic enzyme, or a functional protein. In an aspect a Cas12a system can target any number of nucleic acid molecules, e.g., at least about 2, 3, 4, 5, 10, 25, 50, 75, 100 or more different target nucleic acid molecules.

### Systems

In an aspect, a clustered regularly interspaced short palindromic repeat (CRISPR)-associated (Cas) system is provided. A system can comprise a crRNA comprising a scaffold region and a spacer region capable of hybridizing to a target nucleic acid molecule or gene. The spacer region can comprise one or more 2-aminoadenine nucleotides. The system can further comprise a Cas12a protein or a nucleic acid molecule encoding the Cas12a protein. The nucleic acid molecule encoding the Cas12a protein can be present on a vector. In an aspect, a crRNA is associated with (i.e., bound to) a Cas12a protein to form an RNP complex, which can be delivered to a cell.

### Methods of Modification

Provided herein are methods of targeting (e.g., binding to, modifying, cleaving, detecting, etc.) one or more target nucleic acids (e.g., DNA or RNA) using the Cas12a proteins, crRNA molecules, vectors, and/or Cas12a systems provided herein.

Certain aspects provide methods of modifying a cell (e.g., a eukaryotic cell, a prokaryotic cell, a mammalian cell, a bacterial cell, a primary cell (i.e., a terminally differentiated cell), or any other suitable cell). The methods can comprise delivering to the cell (i) a Cas12a protein or a nucleic acid molecule encoding a Cas12a protein (e.g., a vector encoding a Cas12a protein) and (ii) a crRNA comprising a scaffold region and one or more spacer regions capable of specifically hybridizing to one or more target nucleic acid molecules, wherein the one or more spacer regions comprise one or more 2-aminoadenine nucleotides.

The method can be an in vitro method or an in vivo method. In an aspect, the method can further comprises delivering to the cell a donor nucleic acid molecule. A donor nucleic acid molecule can be a single-stranded donor nucleic acid molecule or a double-stranded donor nucleic acid molecule. A donor nucleic acid molecule can be 5, 10, 20, 30, 40, 50, 60, 70, 80, 90, 100, 200, 500 or more nucleotides in length. A donor nucleic acid molecule can be DNA, RNA, or a hybrid molecule.

A crRNA can comprise 2, 3, 4, 5, 6, 7, or more 2-aminoadenine nucleotides in the spacer region of the crRNA. The method can include multiplexing 2, 3, 4, 5, 6, 7, 8, or more different crRNAs capable of specifically hybridizing to 2, 3, 4, 5, 6, 7, 8, or more target nucleic acid molecules. The target nucleic acid molecules can each be different and matched with the each of the 2 or more crRNA molecules.

The editing efficiency of CRISPR-Cas systems using crRNA molecules with one or more 2-aminoadenine nucleotides in the spacer region more can be improved by 1, 2, 3, 4 fold or 10, 25, 50, 75, 90% or more as compared to a method wherein the crRNA does not comprise one or more 2-aminoadenine nucleotides. In an aspect, CRISPR-Cas systems using crRNA molecules with one or more 2-aminoadenine nucleotides in the spacer region more can have off-target cleavage frequencies that are not increased as compared to a method wherein the crRNA does not comprise one or more 2-aminoadenine nucleotides. In an aspect, CRISPR-Cas systems using crRNA molecules with one or more 2-aminoadenine nucleotides in the spacer region can require a reduced concentration of the Cas12a protein and the one or more crRNAs as compared to a method wherein the crRNA does not comprise one or more 2-aminoadenine nucleotides. The concentration of a Cas12a protein complex associated with a crRNA can be reduced by 2, 3, 4, 5, 6, 7, 8, 9, 10, or more fold as compared to a method wherein the crRNA does not comprise one or more 2-aminoadenine nucleotides.

In an aspect a crRNA can comprise two or more spacer regions such that the Cas12a protein can create a deletion of the one or more target nucleic acid molecules. A direct repeat sequence can separate each spacer in the crRNA.

In some aspects, the Cas12a is delivered to the cell via a nucleic acid molecule encoding the Cas12a protein. The nucleic acid molecule can be present in a vector such as a viral expression vector or a plasmid expression vector, or a linear/circular mRNA molecule.

In some aspects, a Cas12a protein and the one or more crRNAs are allowed to associate into a complex (i.e., a ribonucleoprotein (RNP) complex) prior to delivery to the cell.

In an aspect, the modification is a cell site-specific integration of a donor nucleic acid molecule into a target nucleic acid molecule. A method can comprise: introducing into a cell: (i) a Cas12a protein or a nucleic acid molecule encoding a Cas12a protein; (ii) one or more crRNA molecules comprising a scaffold region and one or more spacer regions capable of hybridizing to the target nucleic acid molecule, wherein the one or more spacer regions comprise one or more 2-aminoadenine nucleotides, and (iii) a donor nucleic acid molecule; wherein the donor nucleic acid molecule is inserted into the target nucleic acid molecule at a site-specific target site.

The donor nucleic acid molecule can include homology arms flanking a sequence for integration into the site-specific target site. The homology arms can comprise a 5′ homology arm and a 3′ homology arm having nucleic acid sequences that are sufficiently similar (e.g., having about 80, 85, 90, 95, 96, 97, 98, 99% or more sequence identity or 100% sequence identity to the nucleic acid sequences located 5′ to the site-specific target site and 3′ to the site-specific target site respectively, to promote homologous recombination.

In an aspect, the donor nucleic acid molecule is a donor polynucleotide comprising an intended edit to be integrated at a site-specific target site in a cell, wherein the donor nucleic acid molecule is flanked by a 5′ homology arm that hybridizes to a sequence 5′ to the site-specific target site and a 3′ homology arm that hybridizes to a sequence 3′ to the site-specific target site. The 5′ homology arm and a 3′ homology arm can have nucleic acid sequences that are sufficiently similar to the nucleic acid sequences located 5′ to the site-specific target site and 3′ to the site-specific target site, respectively, to promote homologous recombination. A homology arm can be from about 20 to about 2,000 nucleotides in length (e.g., about 20, 30, 40, 50, 60, 70, 80, 90, 100, 150, 200, 300, 400, 500, 600, 700, 800, 900, 1,000, 1,500, 2,000, or more nucleotides in length.

A donor nucleic acid molecule can comprise a modified strand comprising two or more nucleic acid modifications to be inserted into a site-specific target site. A donor nucleic acid molecule can be about 50, 75, 100, 250, 500, 750, 1,000, 1,500, 2,000, 3,000, 4,000, 5,000 or more nucleotides in length (in total, including the homology arms)

Cleavage of the genomic DNA at the site-specific target site by Cas12a and subsequent repair of the double stranded break using the donor nucleic acid molecule that includes homology arms by homology-directed repair (HDR) results in integration of sequences of the donor nucleic acid molecule positioned between the homology arms. The method can also be used to simultaneously knock out a gene at the site-specific target site and insert or “knock in” at the disrupted locus a transgene that is provided in the donor nucleic acid molecule. Further provided are methods for inserting a genetic construct at a first site-specific target site, where insertion of the genetic construct knocks out a gene at the first locus, and simultaneously knocking out a gene at a second locus. The knock in/double knock out is achieved by introducing two or more crRNAs into the target cell, a first crRNA having a spacer region targeting the first genetic locus, and a second crRNA having a guide targeting the second genetic locus.

In an aspect a modification results in the reduction of expression of a target gene in a cell. A method of modification can comprise introducing into the cell (a) one or more crRNA molecules comprising a scaffold region and one or more spacer regions capable of hybridizing to the target gene, wherein the one or more spacer regions comprise one or more 2-aminoadenine nucleotides and (b) a Cas12a protein or a nucleic acid molecule encoding a Cas12a protein, wherein the one or more crRNA molecules hybridize to the target gene and the Cas12a cleaves the target gene, such that the target gene expression is reduced in the cell relative to a cell in which the one or more crRNAs and the Cas12a protein or nucleic acid molecule encoding a Cas12a protein are not introduced. In an aspect, the target gene is cleaved at two or more sites. Target gene expression can be reduced by 10, 20, 30, 40, 50, 60, 70, 80, 90, 95, 99% or more.

### Pharmaceutical Compositions

An aspect provides pharmaceutical compositions comprising Cas12a proteins, crRNA molecules, nucleic acid molecules (e.g., mRNA encoding Cas12a proteins), vectors, and/or the Cas12a systems described herein. A pharmaceutical composition can comprise one or more pharmaceutically acceptable excipients or carriers. In an aspect, a pharmaceutical composition comprises one or more Cas12a proteins, one or more crRNAs and one or more pharmaceutically acceptable excipients or carriers. The Cas12a proteins can be associated or hybridized to form RNPs and present in one or more pharmaceutically acceptable excipients or carriers. In an aspect, a pharmaceutical composition comprises one or more Cas12a proteins or nucleic acid molecules encoding one or more Cas12a proteins (e.g., a vector encoding one or more Cas12a proteins), one or more crRNAs, and one or more pharmaceutically acceptable excipients or carriers.

Pharmaceutical compositions suitable for injectable use include, for example, sterile aqueous solutions or dispersions and sterile powders for the extemporaneous preparation of sterile injectable solutions or dispersion. For intravenous administration, suitable excipients include, for example, physiological saline, bacteriostatic water, or phosphate buffered saline. An excipient can be, for example, a solvent or dispersion medium containing, for example, water, ethanol, polyol (for example, glycerol, propylene glycol, and liquid polyethylene glycol, and the like), and suitable mixtures thereof. Proper fluidity of an injectable pharmaceutical composition can be maintained, for example, by the use of a coating such as lecithin, by the maintenance of the required particle size, and by the use of surfactants, e.g., sodium dodecyl sulfate. Antibacterial and antifungal agents, for example, parabens, chlorobutanol, phenol, ascorbic acid, thimerosal, and the like can be included in a pharmaceutical excipient. Other ingredients can include isotonic agents, for example, sugars, polyalcohols such as mannitol, sorbitol, sodium chloride, aluminum monostearate, and gelatin.

Sterile powders for the preparation of sterile injectable solutions, can be made via vacuum drying and freeze-drying yielding a powder of the active ingredient plus any additional desired ingredient from a sterile-filtered solution thereof.

In some aspects, Cas12a proteins, nucleic acid molecules, crRNAs, vectors, or Cas12a systems can be administered by LPN methods, transfection, or infection with nucleic acid molecules encoding them, using methods described in, for example, McCaffrey et al., Nature (2002) 418:6893, Xia et al., Nature Biotechnol (2002) 20:1006-10, and Putnam, Am J Health Syst Pharm (1996) 53:151-60, erratum at Am J Health Syst Pharm (1996) 53:325.

### Methods of Treatment

Pharmaceutical compositions provided herein can be used to treat various disorders (or diseases, symptoms, or pathological conditions). In some aspects, methods for treating a disorder in an individual in need thereof are provided. In some aspects, the methods of treating involves administering a therapeutically effective dose of the pharmaceutical composition provided herein.

A disorder to be treated can be a genetic disorder caused by one or more abnormalities in the genome of an individual. A genetic disorder can be caused by a mutation in a single gene (monogenic) or multiple genes (polygenic) or by a chromosomal abnormality. In some aspects, the disorder is monogenic. In other aspects, the disorder is polygenic.

Methods of treatment can be in the form of a gene therapy. In some aspects, the methods of treating involves modifying one or more target nucleic acids in a cell by introducing into the cell a pharmaceutical composition comprising a Cas12a protein or a nucleic acid molecule encoding a Cas12a protein, nucleic acid molecules, crRNAs, vectors, Cas12a systems or combinations thereof as described herein.

### Kits

In some aspects, kits for carrying out any of the methods described herein are provided. A kit can include one or more components selected from one Cas12a proteins, one or more nucleic acid molecules encoding Cas12a proteins, one or more crRNAs, one or more vectors as described herein, or one or more Cas12a systems as described herein. In an aspect one or more Cas12a proteins are associated with or bound to corresponding crRNA. A kit as described herein can further include one or more additional reagents such as a buffer for introducing one or more Cas12a proteins, one or more nucleic acid molecules encoding Cas12a proteins, one or more crRNAs, one or more vectors as described herein, or one or more Cas12a systems as described herein into a cell, a dilution buffer, a reconstitution solution, a wash buffer, a control reagent, a control expression vector or polyribonucleotide, a reagent for in vitro production of one or more components of the one or more Cas12a proteins, one or more nucleic acid molecules encoding Cas12a proteins, one or more crRNAs, one or more vectors as described herein, or one or more Cas12a systems as described herein and the like. Components of a kit can be in separate containers or can be combined in a single container

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The aspects illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by aspects and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the aspects described in broad terms above.

### EXAMPLES

### Example 1: Methods

### Guide RNA Synthesis by In Vitro Transcription (IVT)

For Cas12a crRNA synthesis, DNA template oligos (contain SP6 promoter sequence, Table 1) for IVT were ordered from IDT (integrated DNA Technologies, Inc.). 5 μM complementary oligos were annealed in 1×NEB buffer 3.0 (New England Biolabs, Inc.) using the following program: 98° C. 5 min, 0.1° C./s cool down to 4° C. HiScribe™ SP6 RNA Synthesis Kit (NEB, E2070S) was used for IVT reactions according to the manufacturer's protocol.

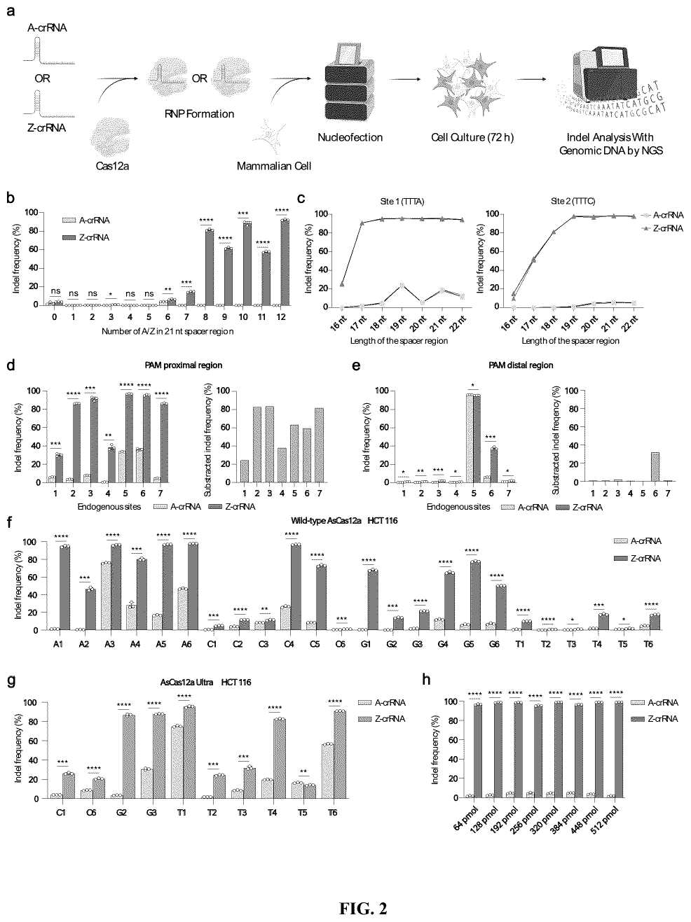

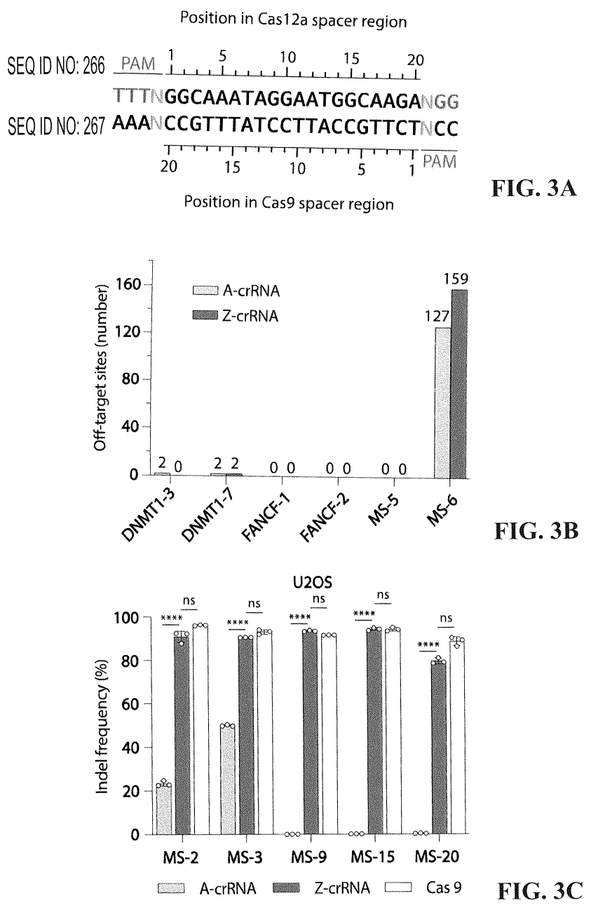

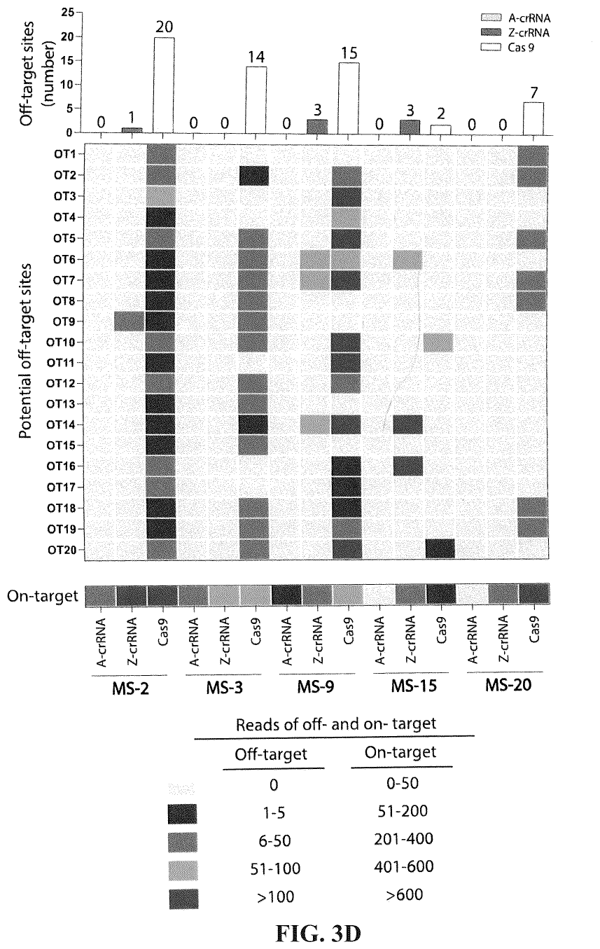

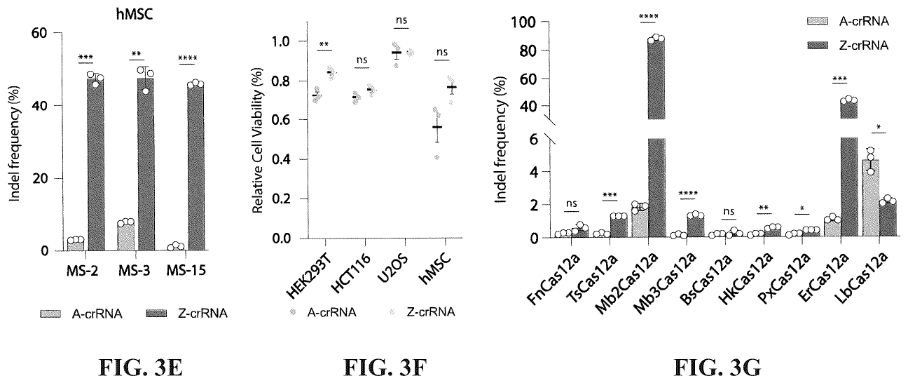

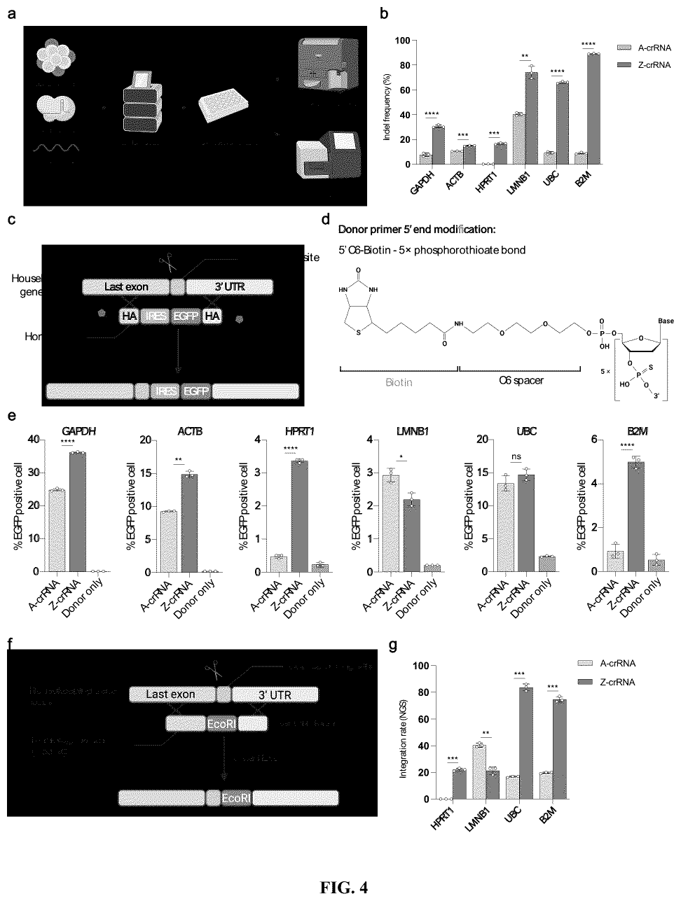

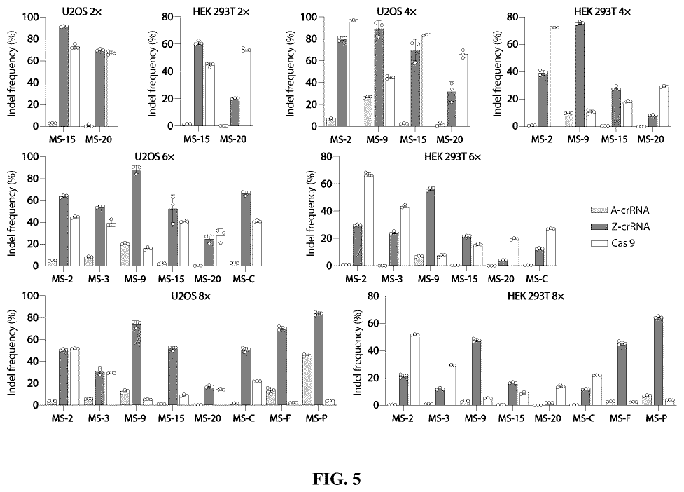

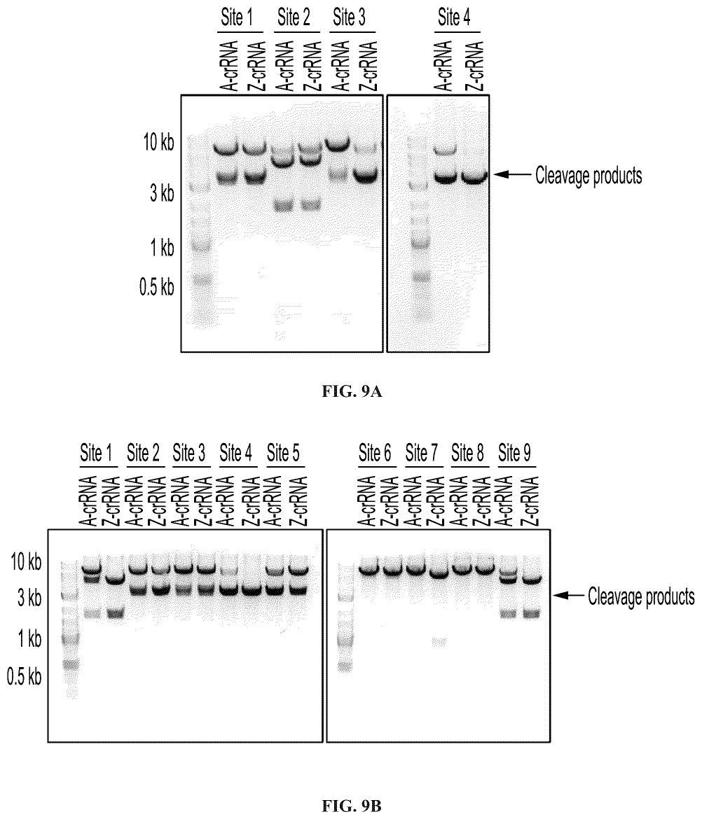

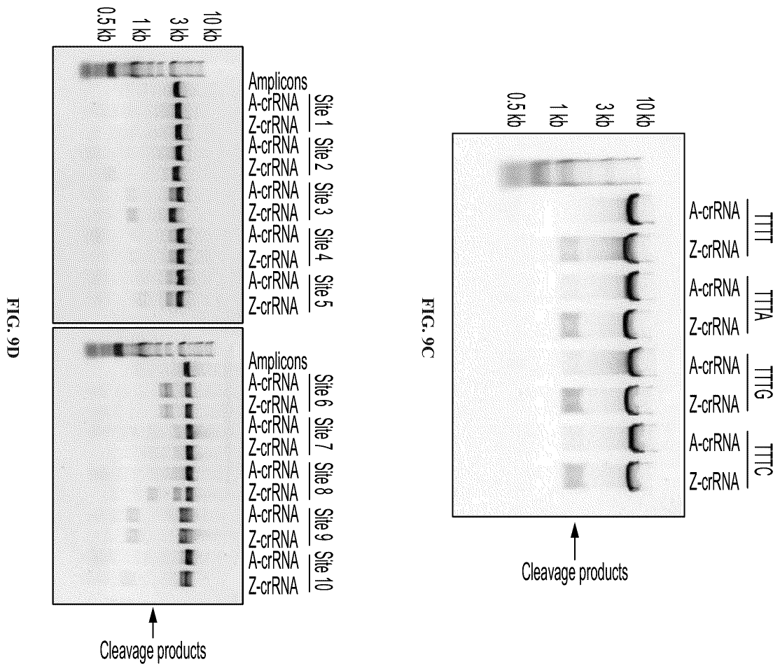

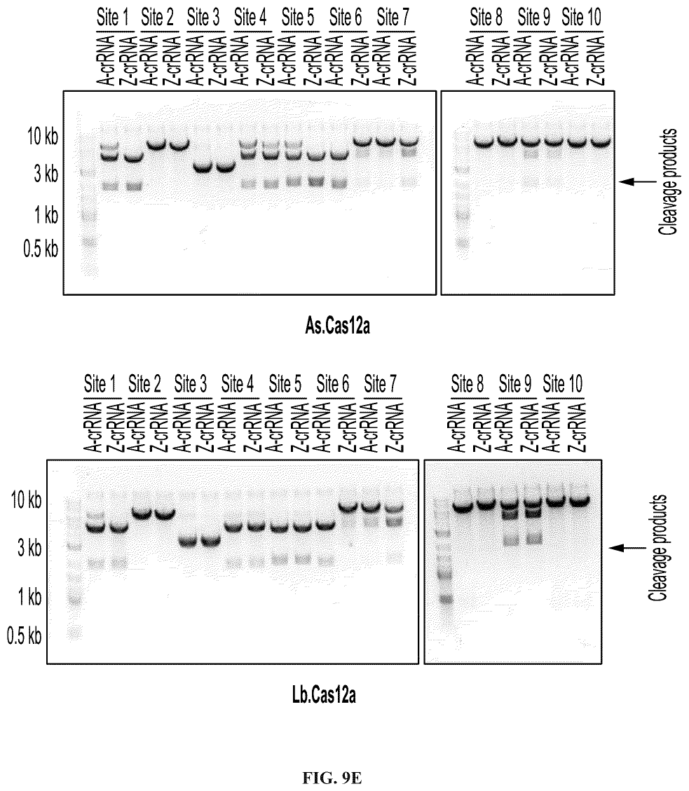

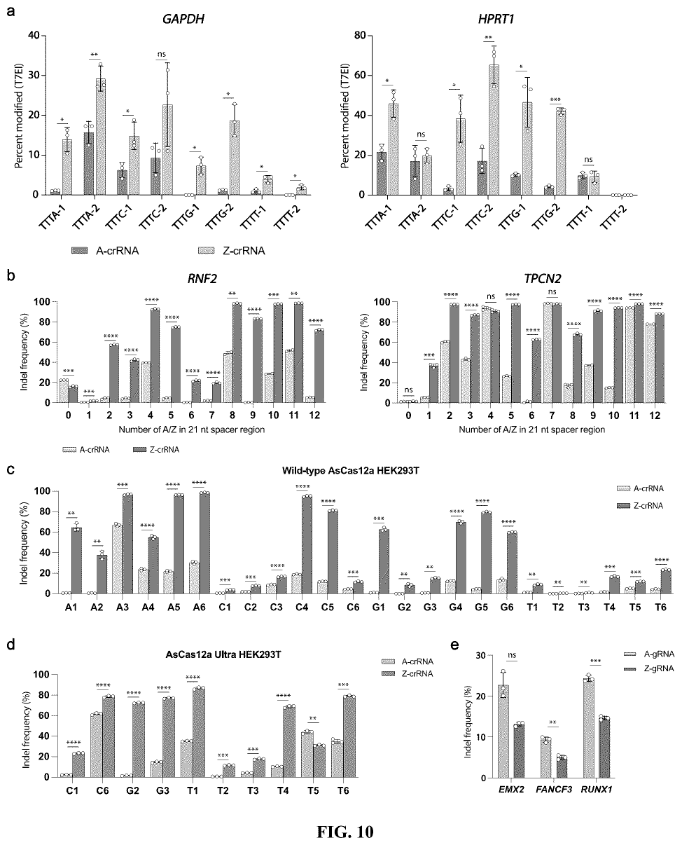

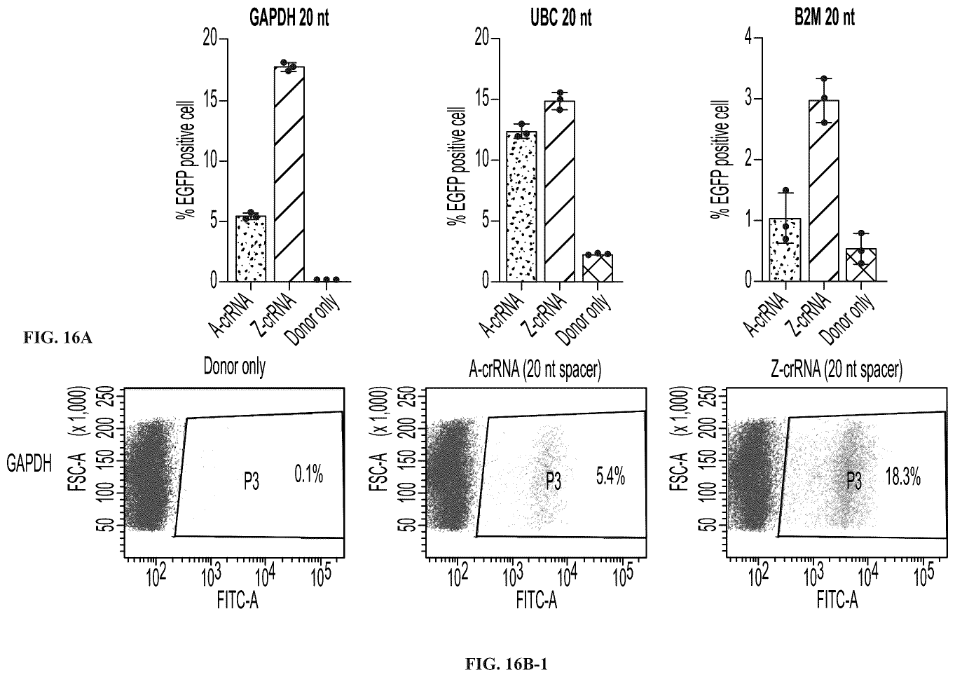

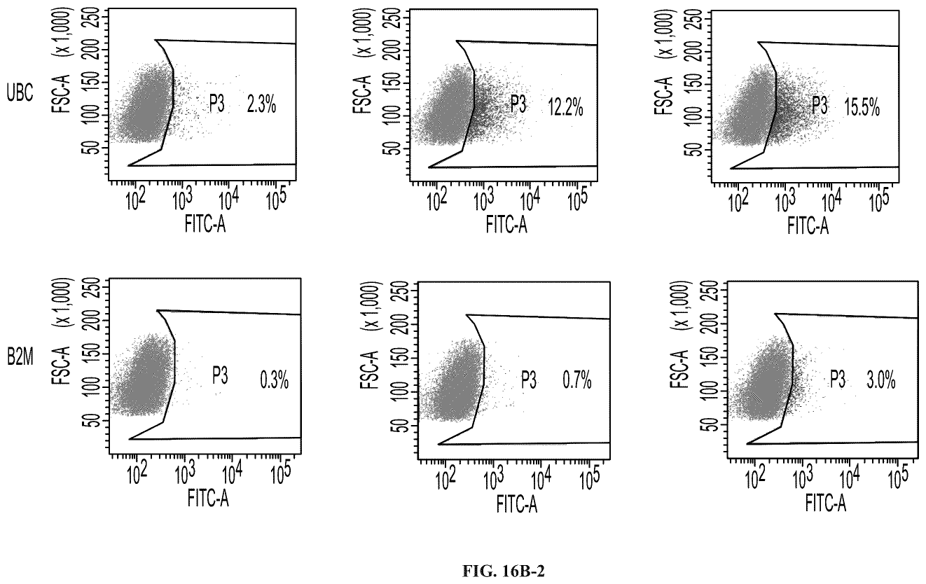

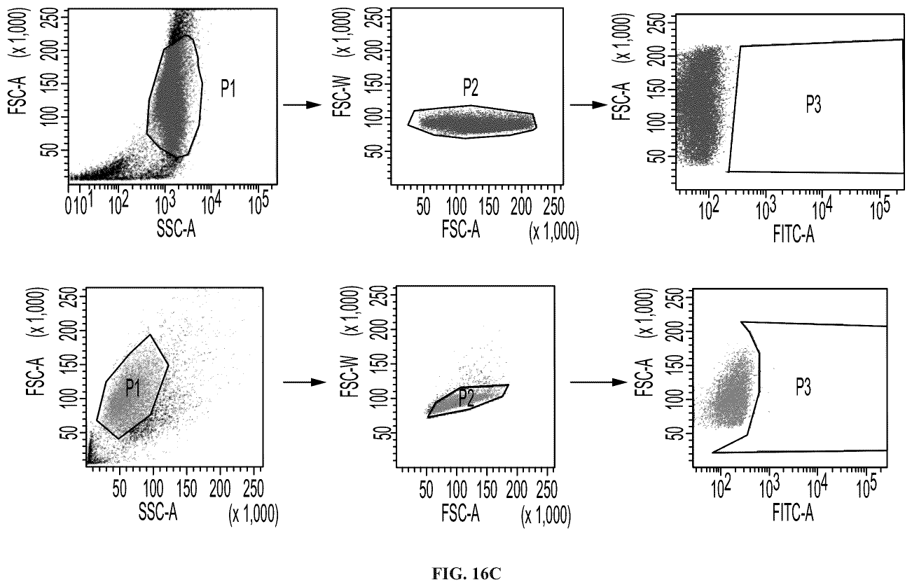

TABLE 1
Detailed sequence information of crRNAs (sgRNAs) used in this study

| | | | | | Chromo-some | |
|---|---|---|---|---|---|---|
| | | Cas12a/ | | | No. and/or | |
| Figure No. | Name | Cas9 | PAM | Spacer sequence 5′-3′ | gene name | Ref. |
| FIG. 1c | 1 NT substitution | Cas12a | TTTG | TTGGAGTTCGTTTTCTTCCTT | chr 2 | This study |
| | | | | (SEQ ID NO: 1) | EMX1 | |
| FIG. 1c | 2 NT substitution | Cas12a | TTTG | TTAGTTCATTTTTCCCTTTGT | chr 2 | This study |
| | | | | (SEQ ID NO: 2) | EMX1 | |
| FIG. 1c | 3 NT substitution | Cas12a | TTTG | TCATCTATTTTACCTTCTGTG | chr 2 | This study |
| | | | | (SEQ ID NO: 3) | EMX1 | |
| FIG. 1c | 4 NT substitution | Cas12a | TTTG | TGTATACGATCAGTTGTGGGG | chr 2 | This study |
| | | | | (SEQ ID NO: 4) | EMX1 | |
| FIG. 1c | 5 NT substitution | Cas12a | TTTG | TACAGGCATCACTTTAGTTTC | chr 2 | This study |
| | | | | (SEQ ID NO: 5) | EMX1 | |
| FIG. 1c | 6 NT substitution | Cas12a | TTTG | CAGATCCAGGCAAGTATCTTG | chr 2 | This study |
| | | | | (SEQ ID NO: 6) | EMX1 | |
| FIG. 1c | 7 NT substitution | Cas12a | TTTG | ACAATTATAAGATCTCTGTTT | chr 2 | This study |
| | | | | (SEQ ID NO: 7) | EMX1 | |
| FIG. 1c | 8 NT substitution | Cas12a | TTTG | AAGACAGGACACGTATTCACC | chr 2 | This study |
| | | | | (SEQ ID NO: 8) | EMX1 | |
| FIG. 1d, | Site-1 | Cas12a | TTTA | TTCTAAATGATAATAAATACT | N/A | This study |
| FIG. 9A | | | | (SEQ ID NO: 9) | | |
| FIG. 1d, | Site-2 | Cas12a | TTTC | TAAATACATTCAAATATGTAT | N/A | This study |
| FIG. 9A | | | | (SEQ ID NO: 10) | | |
| FIG. 1d, | Site-3 | Cas12a | TTTG | ATATCAAAAACTGATTTTCCC | N/A | This study |
| FIG. 9A | | | | (SEQ ID NO: 11) | | |
| FIG. 1d, | Site-4 | Cas12a | TTTG | TTAAAGAGAATTAAGAAAATA | N/A | This study |
| FIG. 9A | | | | (SEQ ID NO: 12) | | |
| FIG. 1d, | Site-5 | Cas12a | TTTT | ACAACACAGAAAGAGTTTGTA | N/A | This study |
| FIG. 9A | | | | (SEQ ID NO: 13) | | |
| FIG. 2b | 0 substitution | Cas12a | TTTG | TCTTCTCTCTGGGTCTGGGCC | chr 2 | This study |
| | | | | (SEQ ID NO: 14) | EMX1 | |
| FIG. 2b | 1 substitution | Cas12a | TTTG | TTGGAGTTCGTTTTCTTCCTT | chr 2 | This study |
| | | | | (SEQ ID NO: 15) | EMX1 | |
| FIG. 2b | 2 substitutions | Cas12a | TTTG | TTAGTTCATTTTTCCCTTTGT | chr 2 | This study |
| | | | | (SEQ ID NO: 16) | EMX1 | |
| FIG. 2b | 3 substitutions | Cas12a | TTTG | TCATCTATTTTACCTTCTGTG | chr 2 | This study |
| | | | | SEQ ID NO: 17 | EMX1 | |
| FIG. 2b | 4 substitutions | Cas12a | TTTG | TGTATACGATCAGTTGTGGGG | chr 2 | This study |
| | | | | SEQ ID NO: 18 | EMX1 | |
| FIG. 2b | 5 substitutions | Cas12a | TTTG | TACAGGCATCACTTTAGTTTC | chr 2 | This study |
| | | | | SEQ ID NO: 19 | EMX1 | |
| FIG. 2b | 6 substitutions | Cas12a | TTTG | CAGATCCAGGCAAGTATCTTG | chr 2 | This study |
| | | | | SEQ ID NO: 20 | EMX1 | |
| FIG. 2b | 7 substitutions | Cas12a | TTTG | ACAATTATAAGATCTCTGTTT | chr 2 | This study |
| | | | | SEQ ID NO: 21 | EMX1 | |
| FIG. 2b | 8 substitutions | Cas12a | TTTG | AAGACAGGACACGTATTCACC | chr 2 | This study |
| | | | | SEQ ID NO: 22 | EMX1 | |
| FIG. 2b | 9 substitutions | Cas12a | TTTG | GAAATAAGGTGTCCTAATGAA | chr 2 | This study |
| | | | | SEQ ID NO: 23 | EMX1 | |
| FIG. 2b | 10 substitutions | Cas12a | TTTG | AGCAAAGCAAATGTACAGGAC | chr 2 | This study |
| | | | | SEQ ID NO: 24 | EMX1 | |
| FIG. 2b | 11 substitutions | Cas12a | TTTG | TAAGGCAAGGAGACATAAAGA | chr 2 | This study |
| | | | | SEQ ID NO: 25 | EMX1 | |
| FIG. 2b | 12 substitutions | Cas12a | TTTG | GAAGAATAACTATATAGAACA | chr 2 | This study |
| | | | | SEQ ID NO: 26 | EMX1 | |
| FIG. 2c | Site-1 16 nt | Cas12a | TTTA | CCAAATTAAGCTGCCT | chr X | This study |
| | | | | SEQ ID NO: 27 | HPRT1 | |
| FIG. 2c | Site-1 17 nt | Cas12a | TTTA | CCAAATTAAGCTGCCTA | chr X | This study |
| | | | | SEQ ID NO: 28 | HPRT1 | |
| FIG. 2c | Site-1 18 nt | Cas12a | TTTA | CCAAATTAAGCTGCCTAA | chr X | This study |
| | | | | SEQ ID NO: 29 | HPRT1 | |
| FIG. 2c | Site-1 19 nt | Cas12a | TTTA | CCAAATTAAGCTGCCTAAT | chr X | This study |
| | | | | SEQ ID NO: 30 | HPRT1 | |
| FIG. 2c | Site-1 20 nt | Cas12a | TTTA | CCAAATTAAGCTGCCTAATG | chr X | This study |
| | | | | SEQ ID NO: 31 | HPRT1 | |
| FIG. 2c | Site-1 21 nt | Cas12a | TTTA | CCAAATTAAGCTGCCTAATGT | chr X | This study |
| | | | | SEQ ID NO: 32 | HPRT1 | |
| FIG. 2c | Site-1 22 nt | Cas12a | TTTA | CCAAATTAAGCTGCCTAATGTT | chr X | This study |
| | | | | SEQ ID NO: 33 | HPRT1 | |
| FIG. 2c | Site-2 16 nt | Cas12a | TTTC | CAGAATATATAAAGAA | chr X | This study |
| | | | | SEQ ID NO: 34 | HPRT1 | |
| FIG. 2c | Site-2 17 nt | Cas12a | TTTC | CAGAATATATAAAGAAA | chr X | This study |
| | | | | SEQ ID NO: 35 | HPRT1 | |
| FIG. 2c | Site-2 18 nt | Cas12a | TTTC | CAGAATATATAAAGAAAC | chr X | This study |
| | | | | SEQ ID NO: 36 | HPRT1 | |
| FIG. 2c | Site-2 19 nt | Cas12a | TTTC | CAGAATATATAAAGAAACA | chr X | This study |
| | | | | SEQ ID NO: 37 | HPRT1 | |
| FIG. 2c | Site-2 20 nt | Cas12a | TTTC | CAGAATATATAAAGAAACAT | chr X | This study |
| | | | | SEQ ID NO: 38 | HPRT1 | |
| FIG. 2c | Site-2 21 nt | Cas12a | TTTC | CAGAATATATAAAGAAACATT | chr X | This study |
| | | | | SEQ ID NO: 39 | HPRT1 | |
| FIG. 2c | Site-2 22 nt | Cas12a | TTTC | CAGAATATATAAAGAAACATTA | chr X | This study |
| | | | | SEQ ID NO: 40 | HPRT1 | |
| FIG. 2d | PAM proximal-1 | Cas12a | TTTC | ATCTATAAAAAGGGGGTGGG | chr 19 | This study |
| | | | | SEQ ID NO: 41 | DNMT1 | |
| FIG. 2d | PAM proximal-2 | Cas12a | TTTC | TAAGAATCAGCGTGTGGTTG | chr 19 | This study |
| | | | | SEQ ID NO: 42 | DNMT1 | |
| FIG. 2d | PAM proximal-3 | Cas12a | TTTA | AAAGACAGTTTTGCTCTGGG | chr 19 | This study |
| | | | | SEQ ID NO: 43 | DNMT1 | |
| FIG. 2d | PAM proximal-4 | Cas12a | TTTG | TTAAAAAGACTGTTTTGGCT | chr 19 | This study |
| | | | | SEQ ID NO: 44 | DNMT1 | |
| FIG. 2d | PAM proximal-5 | Cas12a | TTTA | TAAGAATAATACTGTGCTTC | chr 19 | This study |
| | | | | SEQ ID NO: 45 | DNMT1 | |
| FIG. 2d | PAM proximal-6 | Cas12a | TTTG | CAGTAAGCAAACTGGCTTCC | chr 2 | This study |
| | | | | SEQ ID NO: 46 | EMX1 | |
| FIG. 2d | PAM proximal-7 | Cas12a | TTTG | CCATAAATAAATGCTGCCTG | chr 2 | This study |
| | | | | SEQ ID NO: 47 | EMX1 | |
| FIG. 2e | PAM distal-1 | Cas12a | TTTG | CTGCTGTTCCTGGGCAAAAC | chr 2 | This study |
| | | | | SEQ ID NO: 48 | EMX1 | |
| FIG. 2e | PAM distal-2 | Cas12a | TTTG | TTTTGTTTTTTAATAAGATG | chr 19 | This study |
| | | | | SEQ ID NO: 49 | DNMT1 | |
| FIG. 2e | PAM distal-3 | Cas12a | TTTG | TGCTTTGTTGTGAAACTGAA | chr 19 | This study |
| | | | | SEQ ID NO: 50 | DNMT1 | |
| FIG. 2e | PAM distal-4 | Cas12a | TTTG | TCTGCTTTTTGTATTAAACC | chr 19 | This study |
| | | | | SEQ ID NO: 51 | DNMT1 | |
| FIG. 2e | PAM distal-5 | Cas12a | TTTC | TCCGTTCTGGGGGAAAAAAA | chr 19 | This study |
| | | | | SEQ ID NO: 52 | DNMT1 | |
| FIG. 2e | PAM distal-6 | Cas12a | TTTC | CTGTTCCTCTCAAACAAAGG | chr 2 | This study |
| | | | | SEQ ID NO: 53 | EMX1 | |
| FIG. 2e | PAM distal-7 | Cas12a | TTTC | TCTTCCTTGCACCCAAACAG | chr 2 | This study |
| | | | | SEQ ID NO: 54 | EMX1 | |
| FIG. 2f | a(TTTA) | Cas12a | TTTA | AAAAAGGATTTCCATCTATTA | chr 8 | 1 |
| | BOLL-44-AS | | | SEQ ID NO: 55 | | |
| FIG. 2f | b(TTTA) | Cas12a | TTTA | AAAACTACAACAAACTTACTT | chr 1 | 1 |
| | BRDT-89-AS | | | SEQ ID NO: 56 | | |
| FIG. 2f | c(TTTC) | Cas12a | TTTC | AAAGTTTCCAAACGACCCCAG | chr 1 | 1 |
| | ANKRD45-39-S | | | SEQ ID NO: 57 | | |
| FIG. 2f | d(TTTC) | Cas12a | TTTC | GAAAACCTAAAGAAGAGATAA | chr 1 | 1 |
| | ANKRD50-43-S | | | SEQ ID NO: 58 | | |
| FIG. 2f | e(TTTG) | Cas12a | TTTG | AATCACATTATGGGATTGCTA | chr 2 | 1 |
| | ARL6-26-S | | | SEQ ID NO: 59 | | |
| FIG. 2f | f(TTTG) | Cas12a | TTTG | TACAACGGCTCAAAGAACTGC | chr 4 | 1 |
| | ABHD6-80-S | | | SEQ ID NO: 60 | | |
| FIG. 2f | g(TTTT) | Cas12a | TTTT | AATAGGTAAGTACATCTTTTG | chr 3 | 1 |
| | AHCTF1-89-AS | | | SEQ ID NO: 61 | | |
| FIG. 2f | h(TTTT) | Cas12a | TTTT | ATCACTCCTGGTATATAGAAA | chr 3 | 1 |
| | ADAM7-105-S | | | SEQ ID NO: 62 | | |
| FIG. 2g | EUR-3 C2 | Cas12a | TTTC | CAGAATATATAAAGAAACATT | chr X | This study |
| | | | | SEQ ID NO: 63 | HPRT1 | |
| FIG. 3b | DNMT1 site 3 | Cas12a | TTTC | CTGATGGTCCATGTCTGTTA | chr 19 | 12 |
| | | | | SEQ ID NO: 64 | DNMT1 | |
| FIG. 3b | DNMT1 site 7 | Cas12a | TTTG | GCTCAGCAGGCACCTGCCTC | chr 19 | 12 |
| | | | | SEQ ID NO: 65 | DNMT1 | |
| FIG. 3b | FANCF site 1 | Cas12a | TTTG | GGCGGGGTCCAGTTCCGGGA | chr 11 | 12 |
| | | | | SEQ ID NO: 66 | FANCF | |
| FIG. 3b | FANCF site 2 | Cas12a | TTTG | GTCGGCATGGCCCCATTCGC | chr 11 | 12 |
| | | | | SEQ ID NO: 67 | FANCF | |
| FIG. 3b | Matched site 5 | Cas12a | TTTA | GGATGCCACTAAAAGGGAAA | chr 3 | 12 |
| | | | | SEQ ID NO: 68 | | |
| FIG. 3b | Matched site 6 | Cas12a/ | TTTG/ | GGGTGATCAGACCCAACAGC | chr 3 | 12 |
| | | Cas9 | AGG | SEQ ID NO: 69 | | |
| FIG. 3c, | Matched site 2 | Cas12a/ | TTTG/ | GGCAAATAGGAATGGCAAGA | chr 8 | 12 |
| 3d, 4d | | Cas9 | GGG | SEQ ID NO: 70 | | |
| FIG. 3c, | Matched site 3 | Cas12a/ | TTTG/ | GAGCTGCTTAAGCATTTCAA | chr 11 | 12 |
| 3d, 4d | | Cas9 | GGG | SEQ ID NO: 71 | | |
| FIG. 3c, | Matched site 9 | Cas12a/ | TTTA/ | GGCATGAATTATAATGCTGT | chr 3 | 12 |
| 3d, 4d | | Cas9 | TGG | SEQ ID NO: 72 | | |
| FIG. 3c, | Matched site 15 | Cas12a/ | TTTA/ | GAATCTCAAAAATAAAAGAC | chr 4 | 12 |
| 3d, 4d | | Cas9 | AGG | SEQ ID NO: 73 | | |
| FIG. 3c, | Matched site 20 | Cas12a/ | TTTG/ | GAGAAAATATGGGTTGAGGT | chr 19 | 12 |
| 3d, 4d | | Cas9 | GGG | SEQ ID NO: 74 | | |
| FIG. 4c | GAPDH | Cas12a | TTTA | TTGATGGTACATGACAAG | chr 12 | This study |
| | | | | SEQ ID NO: 75 | | |
| FIG. 4c | ACTB | Cas12a | TTTA | ATAGTCATTCCAAATATG | chr 7 | This study |
| | | | | SEQ ID NO: 76 | | |
| FIG. 4c | HPRT1 | Cas12a | TTTA | AAAGGGAACTGCTGACAA | chr X | This study |
| | | | | SEQ ID NO: 77 | | |
| FIG. 4c | LMNB1 | Cas12a | TTTG | TAATAAGCAATCAAGGTT | chr 5 | This study |
| | | | | SEQ ID NO: 78 | | |
| FIG. 4c | UBC | Cas12a | TTTC | AACAAATTTCATTGCACT | chr 12 | This study |
| | | | | SEQ ID NO: 79 | | |
| FIG. 4c | B2M | Cas12a | TTTA | GAAATATAATTGACAGGA | chr 15 | This study |
| | | | | SEQ ID NO: 80 | | |
| FIG. 4d | Matched site C | Cas12a/ | TTTA/ | GATTCATTCTCAGTGCCATG | chr 3 | 13 |
| | COL8A1 | Cas9 | GGG | SEQ ID NO: 81 | | |
| FIG. 4d | Matched site F | Cas12a/ | TTTA/ | AGAACACATACCCCTGGGCC | chr 5 | 13 |
| | FGF18 | Cas9 | GGG | SEQ ID NO: 82 | | |
| FIG. 4d | Matched site P | Cas12a/ | TTTA/ | CACATAGGCCATTCAGAAAC | chr 17 | 13 |
| | P2RX5-TAX1BP3 | Cas9 | GGG | SEQ ID NO: 83 | | |
| FIG. 9B | Site 1 | Cas12a | TTTT | CAATATTATTGAAGCATTTAT | N/A | This study |
| | | | | SEQ ID NO: 84 | | |
| FIG. 9B | Site 2 | Cas12a | TTTT | CGATTGATGAACACCTATAAT | N/A | This study |
| | | | | SEQ ID NO: 85 | | |
| FIG. 9B | Site 3 | Cas12a | TTTT | CTCATTTATAAGGTTAAATAA | N/A | This study |
| | | | | SEQ ID NO: 86 | | |
| FIG. 9B | Site 4 | Cas12a | TTTT | GTATATACAATATTTCTAGTT | N/A | This study |
| | | | | SEQ ID NO: 87 | | |
| FIG. 9B | Site 5 | Cas12a | TTTT | GATATCAAAATTATACATGTC | N/A | This study |
| | | | | SEQ ID NO: 88 | | |
| FIG. 9B | Site 6 | Cas12a | TTTT | ATTAGGAAAGGACAGTGGGAG | N/A | This study |
| | | | | SEQ ID NO: 89 | | |
| FIG. 9B, | Site 7 | Cas12a | TTTT | AACCAATAGGCCGAAATCGGC | N/A | This study |
| 3c | | | | SEQ ID NO: 90 | | |
| FIG. 9B | Site 8 | Cas12a | TTTT | GCAAAAAGCTCCCGGGAGCTT | N/A | This study |
| | | | | SEQ ID NO: 91 | | |
| FIG. 9B | Site 9 | Cas12a | TTTT | CTAAATACATTCAAATATGTA | N/A | This study |
| | | | | SEQ ID NO: 92 | | |
| FIG. 9D | Site 1 | Cas12a | TTTT | GGCGCCAAACAGCCCGGGCAC | N/A | This study |
| | | | | SEQ ID NO: 93 | | |
| FIG. 9D | Site 2 | Cas12a | TTTT | GATCCCCAAATACAGCAAGCT | N/A | This study |
| | | | | SEQ ID NO: 94 | | |
| FIG. 9D | Site 3 | Cas12a | TTTT | GGCAGGAACTTTAACCGTGCA | N/A | This study |
| | | | | SEQ ID NO: 95 | | |
| FIG. 9D | Site 4 | Cas12a | TTTT | CCTGTAAATCTGTGCCTGTGA | N/A | This study |
| | | | | SEQ ID NO: 96 | | |
| FIG. 9D | Site 5 | Cas12a | TTTT | CAGGAATGAACTGATGGCGTT | N/A | This study |
| | | | | SEQ ID NO: 97 | | |
| FIG. 9D | Site 6 | Cas12a | TTTT | GAGACAGGGTCTCACTCTGTC | N/A | This study |
| | | | | SEQ ID NO: 98 | | |
| FIG. 9D | Site 7 | Cas12a | TTTT | GGGAGAATACTGGCACAGATG | N/A | This study |
| | | | | SEQ ID NO: 99 | | |
| FIG. 9D | Site 8 | Cas12a | TTTT | AAATCAAGTTTTATTTTGGGA | N/A | This study |
| | | | | SEQ ID NO: 100 | | |
| FIG. 9D | Site 9 | Cas12a | TTTT | AAGAGACAGGATCTCACTGTG | N/A | This study |
| | | | | SEQ ID NO: 101 | | |
| FIG. 9D | Site 10 | Cas12a | TTTT | AAGGATTAACTTGGGCATGGT | N/A | This study |
| | | | | SEQ ID NO: 102 | | |
| FIG. 9E | Site 1 | Cas12a | TTTC | GAGATTTATTTTCTTAATTCT | N/A | This study |
| | | | | SEQ ID NO: 103 | | |
| FIG. 9E | Site 2 | Cas12a | TTTG | TTAAATCAGCTCATTTTTTAA | N/A | This study |
| | | | | SEQ ID NO: 104 | | |
| FIG. 9E | Site 3 | Cas12a | TTTA | TTTTTCTAAATACATTCAAAT | N/A | This study |
| | | | | SEQ ID NO: 105 | | |
| FIG. 9E | Site 4 | Cas12a | TTTA | TTTTCTTAATTCTCTTTAACA | N/A | This study |
| | | | | SEQ ID NO: 106 | | |
| FIG. 9E | Site 5 | Cas12a | TTTC | ACAAATAAAGCATTTTTTTCA | N/A | This study |
| | | | | SEQ ID NO: 107 | | |
| FIG. 9E | Site 6 | Cas12a | TTTA | TGATTTTTTGTATATACAATA | N/A | This study |
| | | | | SEQ ID NO: 108 | | |
| FIG. 9E | Site 7 | Cas12a | TTTA | TTATTTTCGAGATTTATTTTC | N/A | This study |
| | | | | SEQ ID NO: 109 | | |
| FIG. 9E | Site 8 | Cas12a | TTTG | ATTTATAAGGGATTTTGCCGA | N/A | This study |
| | | | | SEQ ID NO: 110 | | |
| FIG. 9E | Site 9 | Cas12a | TTTG | TGAAATTTGTGATGCTATTGC | N/A | This study |
| | | | | SEQ ID NO: 111 | | |
| FIG. 9E | Site 10 | Cas12a | TTTC | CTCATTTTATTAGGAAAGGAC | N/A | This study |
| | | | | SEQ ID NO: 112 | | |
| FIG. 10 | GAPDH TTTA-1 | Cas12a | TTTA | ATAATAATGAAACTGGCGAGT | chr 12 | This study |
| | | | | SEQ ID NO: 113 | GAPDH | |
| FIG. 10 | GAPDH TTTA-2 | Cas12a | TTTA | GCTGATACTTAAACAGAGACC | chr 12 | This study |
| | | | | SEQ ID NO: 114 | GAPDH | |
| FIG. 10 | GAPDH TTTC-1 | Cas12a | TTTC | ATTATTATTAAAGAATCCATT | chr 12 | This study |
| | | | | SEQ ID NO: 115 | GAPDH | |
| FIG. 10 | GAPDH TTTC-2 | Cas12a | TTTC | CAAGAATCAGGGACACTGTAG | chr 12 | This study |
| | | | | SEQ ID NO: 116 | GAPDH | |
| FIG. 10 | GAPDH TTTG-1 | Cas12a | TTTG | TAATTTTAGTAGAGACGGAGT | chr 12 | This study |
| | | | | SEQ ID NO: 117 | GAPDH | |
| FIG. 10 | GAPDH TTTG-2 | Cas12a | TTTG | AATGTCAGCTCAACACAGCCT | chr 12 | This study |
| | | | | SEQ ID NO: 118 | GAPDH | |
| FIG. 10 | GAPDH TTTT-1 | Cas12a | TTTT | AGTAGAGACGGAGTITCACCA | chr 12 | This study |
| | | | | SEQ ID NO: 119 | GAPDH | |
| FIG. 10 | GAPDH TTTT-2 | Cas12a | TTTT | CCAAGAATCAGGGACACTGTA | chr 12 | This study |
| | | | | SEQ ID NO: 120 | GAPDH | |
| FIG. 10 | HPRT1 TTTA-1 | Cas12a | TTTA | CCAAATTAAGCTGCCTAATGT | chr X | This study |
| | | | | SEQ ID NO: 121 | HPRT1 | |
| FIG. 10 | HPRT1 TTTA-2 | Cas12a | TTTA | GGATTAGTACGGATCAGCCAG | chr X | This study |
| | | | | SEQ ID NO: 122 | HPRT1 | |
| FIG. 10 | HPRT1 TTTC-1 | Cas12a | TTTC | CATTTTACAGTTTTACCAAAT | chr X | This study |
| | | | | SEQ ID NO: 123 | HPRT1 | |
| FIG. 10 | HPRT1 TTTC-2 | Cas12a | TTTC | CAGAATATATAAAGAAACATT | chr X | This study |
| | | | | SEQ ID NO: 124 | HPRT1 | |
| FIG. 10 | HPRT1 TTTG-1 | Cas12a | TTTG | CAGCATCAATAACATTGATGT | chr X | This study |
| | | | | SEQ ID NO: 125 | HPRT1 | |
| FIG. 10 | HPRT1 TTTG-2 | Cas12a | TTTG | AAACAGTGAGTTAAAATCTGG | chr X | This study |
| | | | | SEQ ID NO: 126 | HPRT1 | |
| FIG. 10 | HPRT1 TTTT-1 | Cas12a | TTTT | AGAGGCTGAGGCAGGAAGACT | chr X | This study |
| | | | | SEQ ID NO: 127 | HPRT1 | |
| FIG. 10 | HPRT1 TTTT-2 | Cas12a | TTTT | AAATTGTTTGCAGCATCAATA | chr X | This study |
| | | | | SEQ ID NO: 128 | HPRT1 | |
| FIG. 16A- | GAPDH | Cas12a | TTTA | TTGATGGTACATGACAAGGT | chr 12 | This study |
| 16C | | | | SEQ ID NO: 129 | | |
| FIG. 16A- | UBC | Cas12a | TTTC | AACAAATTTCATTGCACTTT | chr 12 | This study |
| 16C | | | | SEQ ID NO: 130 | | |
| FIG. 16A- | B2M | Cas12a | TTTA | GAAATATAATTGACAGGATT | chr 15 | This study |
| 16C | | | | SEQ ID NO: 131 | | |

For Cas12a crRNA synthesis, the following sequence should be added at the 5′ of the spacer sequence mentioned above.
AATTTAGGTGACACTATAGTAATTTCTACTCTTGTAGAT + Spacer SP6 promoter; Direct repeat of crRNA (SEQ ID NO: 132)
For Cas9 sgRNA synthesis, we followed the manufacture′s protocol (Precision gRNA Synthesis Kit (Thermo Scientific, A29377))

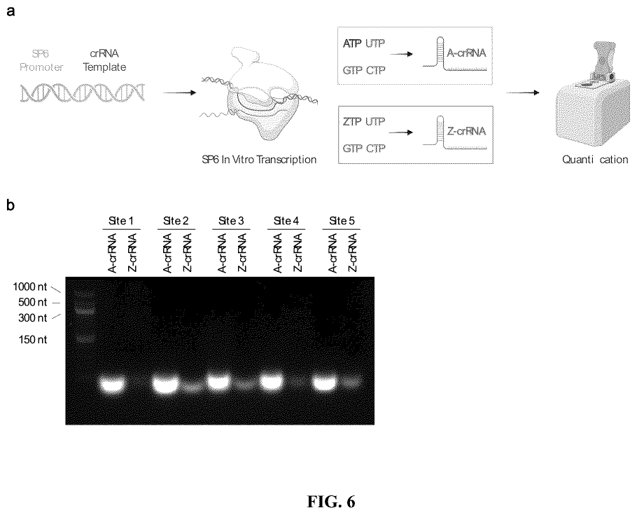

To generate Z-incorporated crRNA, ZTP (TriLink Biotechnologies, N-1001, 2-Amino-ATP) was used to replace ATP in the reaction (FIG. 6a). Reactions were incubated at 37° C. for 10 hours to reach higher concentration. For Cas9 sgRNA synthesis, Precision gRNA Synthesis Kit (Thermo Scientific, A29377) was used to create the template DNA for IVT. DNA oligos were ordered from IDT as per kit's instruction. To remove DNA template, 25 μL of nuclease-free water with 2 μL of DNase I (NEB, M0303S) were added to each 25 μL reaction, and the reactions were incubated at 37° C. for 15 minutes. Synthesized RNAs were purified using RNA Clean & Concentrator-5 kit (Zymo, R1016), and the concentrations were quantified by the NanoDrop 2000/2000c (Thermo Scientific) and Qubit 4 fluorometer. RNAs were stored at −80° C. until use.

### In Vitro Cleavage Assay

The synthesized crRNAs were diluted to 10 μM with nuclease-free water. Diluted crRNAs were pre-incubated with AsCas12a proteins (IDT, 1081069) or LbCas12a proteins (IDT, 10007922) to form the ribonucleoproteins (RNPs) in the 1×NEB 2.0 buffer using a 1:1.2 ratio of Cas12a:crRNA. The final concentration of RNP was designed to 1 μM. After 15 min incubation at room temperature, the RNPs were mixed with 1 μL 10× Reaction Buffer (NEB 2.0), 1 μg purified plasmid (Table 3), 10 U NdeI restriction enzyme, and added with nuclease-free water up to 10 μL. The reaction was incubated at 37° C. for 30 min. The digestion products were analyzed by running 1% agarose gel. For kinetics study, 10 nM dsDNA substrate was incubated with various amount of A/Z-AsCas12a RNPs (20 nM, 40 nM, 100 nM, and 500 nM). Cleavage reactions were sampled at distinct time points (10 s, 20 s, 60 s, 100 s, and 600 s), and substrate cleavage was assessed through capillary electrophoresis (Agilent).

### Annealing Temperature (Ta) Measurement

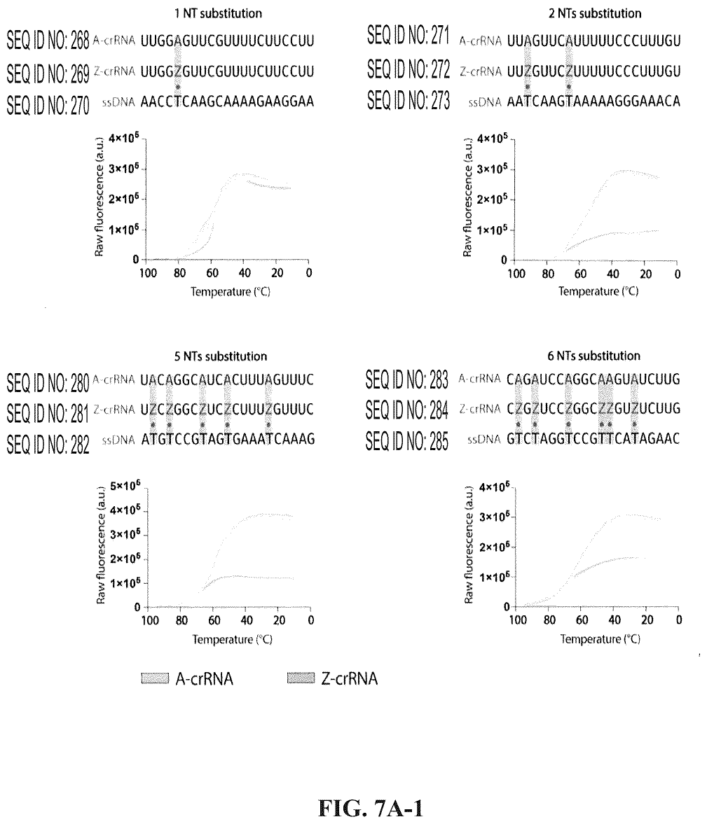

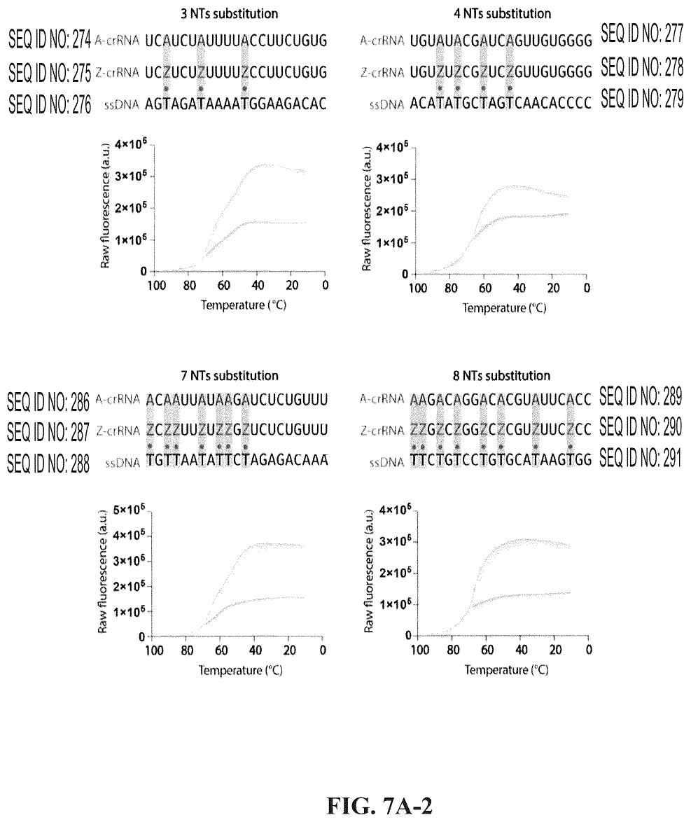

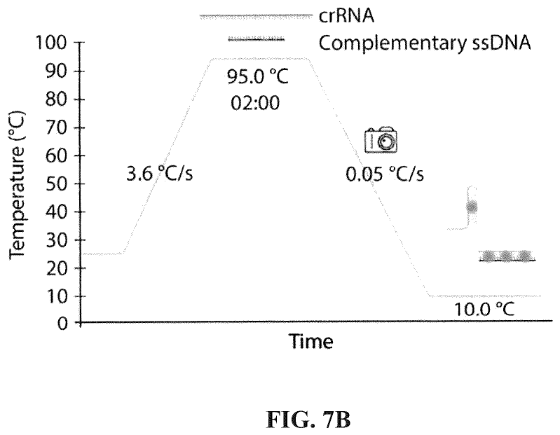

The synthesized crRNAs with various A/Z base content and their complementary ssDNAs were diluted to 10 μM. 1 μL of each crRNA and its complementary ssDNA were added in a 10 μL solution with 2× EvaGreen (Biotium, 31000-T), 10 mM phosphate (pH 7.4), and 100 mM NaCl. QuantStudio™ 3 Real-Time PCR System was used to record the raw fluorescence during the annealing step with the following program: 95° C. 2 min, cool down to 10° C. with the ramp of 0.05° C./s, fluorescence was measured every 0.05 s (FIG. 7b). Ta values were calculated as follows:

$$crRNA - ssDNA binary complex Ta = \frac{\overline{{T}_{{A}_{min}}} - \overline{{T}_{{A}_{max}}}}{2}$$

where Ta represents the temperature of annealing (Ta), and TA represents the raw fluorescence associated temperature.

### Microscale Thermophoresis (MST) Assays

50 nt DNA oligonucleotides and their complementary strands were synthetized at the 250 nmole scale (IDT, HPLC 95% pure). For dsDNA formation, the oligonucleotides were dissolved in duplex buffer at a concentration of 100 μM. Equal volumes of complementary strands were then combined, heated to 95° C. for 5 minutes, and subsequently cooled to 4° C. To remove any ssDNA, the mixture was treated with Exonuclease (New England Biolabs). Purification of the dsDNA was achieved using the Monarch® PCR & DNA Cleanup Kit (5 μg, New England Biolabs). The concentration of the dsDNA was determined using an Invitrogen™ Qubit™ 4 Fluorometer.

The binding affinity between AsCas12a and DNA was measured using Monolith NT.115 (Nanotemper Technologies). AsCas12a was fluorescently labelled using the Monolith Protein Labeling Kit RED-NHS 2nd Generation (Amine Reactive, product number MO-L011). A volume of 90 μL AsCas12a sample (10 μM) in the labelling buffer was mixed with 10 μL dye solution (300 μM) for 30 min at room temperature in the dark. Next, the AsCas12a sample was loaded to column B (Nanotemper MO-L011) and eluted with 450 μL of assay buffer (50 mM Tris-HCl, pH 7.4, 150 mM NaCl, 5 mM EDTA, 5 mg/mL BSA, 0.05% Tween-20). The labelled AsCas12a sample was incubated with crRNA at a ratio of 1:1.2 to form the AsCas12a-A-crRNA RNP or AsCas12a-Z-crRNA RNP. To perform the MST assay, we first combined 10 μL of the labeled sample with 10 μL of DNA. This DNA was present in 16 varying serial dilutions in the assay buffer, allowing the reaction to proceed for 5 minutes to permit binding. The samples were then loaded into Monolith NT.115 capillary (Nanotemper Technologies) and measured by using 60% RED as the excitation power and a medium MST power setting. The binding measurement was repeated three times. Data analysis was performed using Nanotemper affinity analysis software.

### Mammalian Cell Culture and Nucleofection

HCT116 cells (ATCC, CCL-247) were cultured in McCoy's 5A medium supplemented with 10% FBS. HEK293T (ATCC, CRL-3216) and U2OS (ATCC, HTB-96) cells were cultured in Advanced DMEM supplemented with 10% FBS and 1×GlutaMax (ThermoFisher, 35050061). Primary human Mesenchymal Stem Cells (hMSC) was obtained from ATCC (PSC-500-012) and cultured in Mesenchymal Stem Cell Basal Medium (PSC-500-030) supplemented with Bone Marrow-Mesenchymal Stem Cell Growth Kit Components (PSc-500-041). All cells were cultured at 37° C. in a 5% C02 incubator. To form the RNP, nuclease and crRNA (sgRNA) were diluted with PBS to a total volume of 10 μL followed by the incubation at room temperature for 10-20 min. For U2OS and HCT116 cell lines, 1×10⁶ cells were transfected using SE Cell Line Nucleofector Kit with RNP containing 320 pmol crRNA and 192 pmol AsCas12a Nuclease (IDT, 1081069), AsCas12a Ultra Nuclease (IDT, 10001273), or LbCas12a Nuclease (IDT, 10007923) using DN-100 program and EN-113 program respectively on a Lonza 4D-Nucleofector according to manufacturer's instructions. 1×10⁶ HEK293T cells were transfected using SF Cell Line nucleofector Kit with RNP complex containing 320 pmol crRNA and 192 pmol AsCas12a Nuclease or AsCas12a Ultra Nuclease using DS-150 program. All nucleofections were supplemented with 300 pmol Cas12a Electroporation Enhancer (IDT, 1076301). Cas12a orthologs validation was conducted on HEK293T cells, as previously described, involving FnCas12a, TsCas12a, Mb2Cas12a, Mb3Cas12a, BsCas12a, HkCas12a, PxCas12a, and ErCas12a. For hMSCs, 0.5×10⁶ cells were transfected using P1 Primary Cell Nucleofector Kit with RNP containing 320 pmol crRNA and 192 pmol AsCas12a Nuclease (IDT, 1081069) using FF-104 on a Lonza 4D-Nucleofector according to manufacturer's instructions. For Cas9 based genome editing experiments, U2OS cells were transfected using SE Cell Line Nucleofector Kit with RNP containing 320 pmol sgRNA and 192 pmol SpCas9 Nuclease (IDT, 1081059) using DN-100 program on a Lonza 4D-Nucleofector according to manufacturer's instructions. All nucleofections were supplemented with 300 pmol Cas9 EIectroporation Enhancer (IDT, 1075916). Genomic DNA was extracted 72 hours after nucleofection using the Quick-DNA Miniprep Kit (Zymo, R1055). Genomic DNAs were stored at −20° C. until use.

### Cell Viability Assay

1×10⁶ HEK293T/HCT116/U2OS/hMSC cells were transfected with A-crRNA-RNP and Z-crRNA-RNP by nucleofection respectively. 48 hrs after nucleofection, culture media was carefully aspirated away and 200 ul PBS was added per well followed by equal volume of the CellTiter-Glo reagent (Promega, G7570). The reagents were mixed for 2 minutes on an orbital shaker to induce cell lysis and then the plate was incubated at room temperature for 10 minutes before measurement. Luminescence signal was detected with the SpectraMax M5 plate reader (Molecular Devices) with an integration time of 1000 ms. The absolute luminescence signal of each well was then normalized to the average signal of control group (cells without nucleofection) to obtain the relative cell viability.

### GUIDE-Seq

U2OS cell line was used for GUIDE-Seq experiments. 1×10⁶ cells were transfected using SE Cell Line Nucleofector Kit with RNP containing 320 pmol crRNA and 192 pmol Cas12a Nuclease (or 320 pmol sgRNA and 192 pmol Cas9 Nuclease) using DN-100 program. RNP and 100 pmol of end-protected double-stranded oligodeoxynucleotide (dsODN) containing an NdeI restriction site were co-transfected into U2OS cells. Genomic DNA was extracted 72 hours after nucleofection using a Quick-DNA Miniprep Kit. Targeted editing sites were amplified by specific primers sets accordingly using KOD Xtreme Hot Start DNA Polymerase (Sigma-Aldrich, 71975-3). NdeI digestion was performed to determine the integration efficiency to ensure the GUIDE-Seq experiment is valid (integration rate>10%). 20 μL digestion reaction contains 2 μL 10×NEB CutSmart buffer, 20 U NdeI, and 200 ng PCR product. The reaction was incubated at 37° C. for 1 hour. Restriction-fragment length polymorphism (RFLP) assay was performed as described elsewhere⁴⁴. For the creation of sequencing libraries, we followed the published protocol⁵⁶ with minor modifications: the sheared genomic DNAs were end-repaired by using NEBNext© Ultra™ II End Repair/dA-Tailing Module (NEB, E7546S); The barcoded Y-adapter was ligated to the repaired genomic DNA by utilizing NEBNext® Ultra™ II Ligation Module (NEB, E7595S). High-throughput sequencing libraries were generated after tag-specific amplification, and the libraries were sequenced using an Illumina MiSeq sequencer. Data were analyzed using open-source software⁴⁴. Un-demultiplex FASTQ sequence data (paired end reads along with dual-index sample barcodes) were processed using a GUIDE-Seq Python-based workflow (github.com/aryeelab/guideseq, commit: 190053c3).

### T7EI Assays

T7 endonuclease I (T7EI) mutation detection kit (IDT, 1075932) was used to determine Cas12a editing efficiencies. The targeted loci were amplified from genomic DNA using KOD Xtreme Hot Start DNA Polymerase with 20 ng of genomic DNA as template. PR products were denatured and followed by annealing with the addition of T7EI reaction buffer as perCthe manufacture's protocol. The annealed PR products were digested with Ti endonuclease I at 37 AC for 1 hour. Fragment analyzer (Agilent) was used to estimate the modification percentages.

### Targeted Deep Sequencing by NGS

On-target sites were amplified from genomic DNA using KOD Xtreme Hot Start DNA Polymerase with primers containing Illumina adaptors (Table 2) overhang nucleotide sequences (Illumina Nextera XT Index Kit v2 Set A, FC-131-2001). KAPA HiFi HotStart ReadyMix (Roche, KK2602) was used to generate dual-indexed sequencing libraries. The libraries were sequenced by Illumina MiSeq-Nano. OutKnocker 2.0 beta (outknocker.org/outknocker2.htm) was used to analyze the indel frequency (with 2% allele threshold).

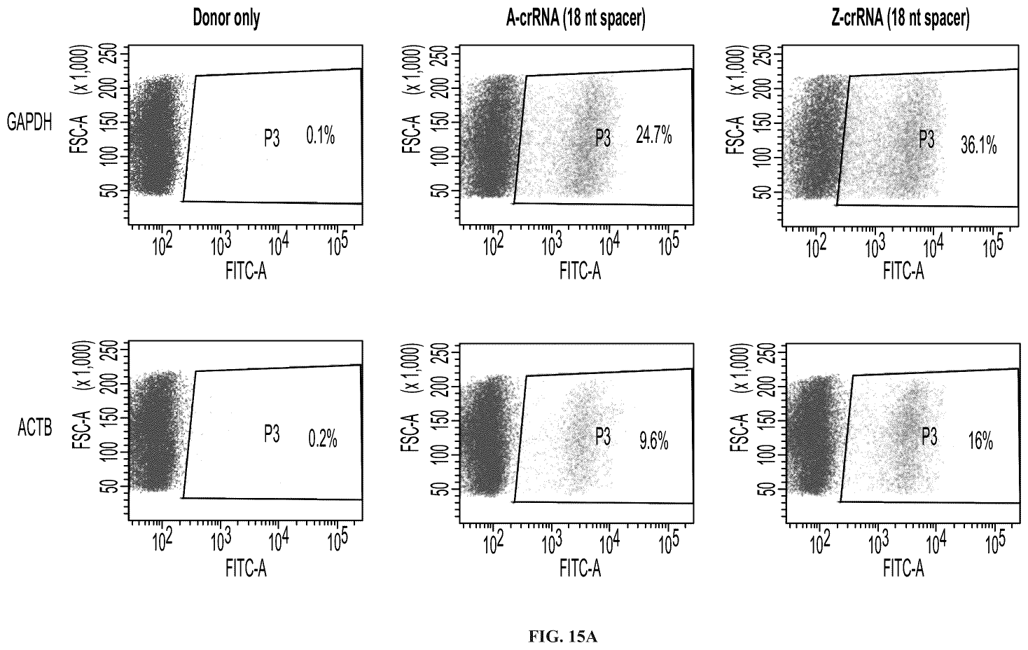

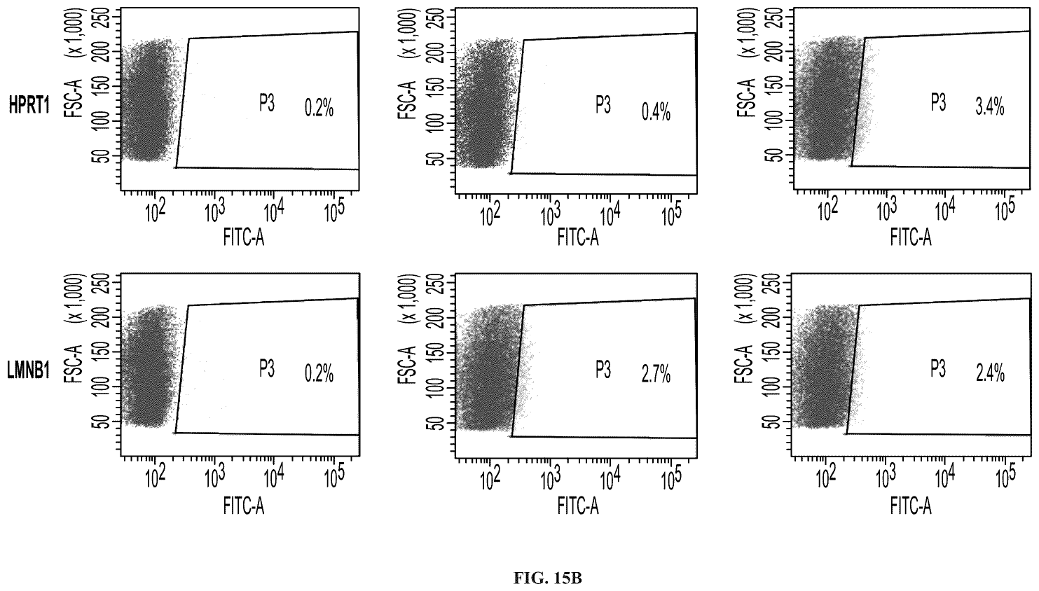

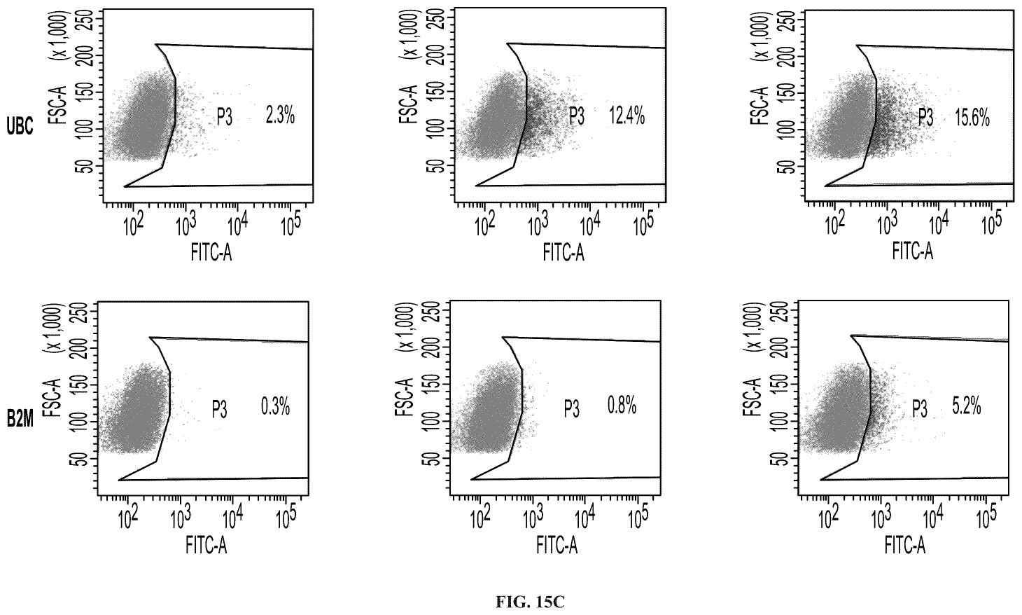

TABLE 2
Sequence information of the oligos used in this study

| Figure No. | Primer name | Sequence 5′-3′ | Use |
|---|---|---|---|
| FIG. 1c | 1 NT substitution | AAGGAAGAAAACGAACTCCAA SEQ ID NO: 135 | Annealing to the |
| | | | crRNA |
| FIG. 1c | 2 NTs substitution | ACAAAGGGAAAAATGAACTAA SEQ ID NO: 136 | Annealing to the |
| | | | crRNA |
| FIG. 1c | 3 NTs substitution | CACAGAAGGTAAAATAGATGA SEQ ID NO: 137 | Annealing to the |
| | | | crRNA |
| FIG. 1c | 4 NTs substitution | CCCCACAACTGATCGTATACA SEQ ID NO: 138 | Annealing to the |
| | | | crRNA |
| FIG. 1c | 5 NTs substitution | GAAACTAAAGTGATGCCTGTA SEQ ID NO: 139 | Annealing to the |
| | | | crRNA |
| FIG. 1c | 6 NTs substitution | CAAGATACTTGCCTGGATCTG SEQ ID NO: 140 | Annealing to the |
| | | | crRNA |
| FIG. 1c | 7 NTs substitution | AAACAGAGATCTTATAATTGT SEQ ID NO: 141 | Annealing to the |
| | | | crRNA |
| FIG. 1c | 8 NTs substitution | GGTGAATACGTGTCCTGTCTT SEQ ID NO: 142 | Annealing to the |
| | | | crRNA |
| FIG. 2b | Deep-Seq-[A]-0-F | TCGTCGGCAGCGTCAGATGTGTATAAGAGACAGGTGTTTCCA | NGS |
| | | CCATTCATCTCA SEQ ID NO: 143 | |
| FIG. 2b | Deep-Seq-[A]-0-R | GTCTCGTGGGCTCGGAGATGTGTATAAGAGACAGGTGGAGG | NGS |
| | | AGGTAGTATACAGA SEQ ID NO: 144 | |
| FIG. 2b | Deep-Seq-[A]-1-F | TCGTCGGCAGCGTCAGATGTGTATAAGAGACAGTCGGCTTGC | NGS |
| | | TAATAAAGATCC SEQ ID NO: 145 | |
| FIG. 2b | Deep-Seq-[A]-1-R | GTCTCGTGGGCTCGGAGATGTGTATAAGAGACAGTGTGGTCT | NGS |
| | | GGTCTACACTAAC SEQ ID NO: 146 | |
| FIG. 2b | Deep-Seq-[A]-2-F | TCGTCGGCAGCGTCAGATGTGTATAAGAGACAGGAGTGGTGA | NGS |
| | | AAAAGAAGGGATC SEQ ID NO: 147 | |
| FIG. 2b | Deep-Seq-[A]-2-R | GTCTCGTGGGCTCGGAGATGTGTATAAGAGACAGAGAAGAA | NGS |
| | | GGGTTGGCTTGA SEQ ID NO: 148 | |
| FIG. 2b | Deep-Seq-[A]-3-F | TCGTCGGCAGCGTCAGATGTGTATAAGAGACAGCTTCTGTCTG | NGS |
| | | CTCTCATTAAACTG SEQ ID NO: 149 | |
| FIG. 2b | Deep-Seq-[A]-3-R | GTCTCGTGGGCTCGGAGATGTGTATAAGAGACAGAGACAAAG | NGS |
| | | CCAGAGCCAATA SEQ ID NO: 150 | |
| FIG. 2b | Deep-Seq-[A]-4-F | TCGTCGGCAGCGTCAGATGTGTATAAGAGACAGGAAGAGCCT | NGS |
| | | CCAGTCTCTG SEQ ID NO: 151 | |
| FIG. 2b | Deep-Seq-[A]-4-R | GTCTCGTGGGCTCGGAGATGTGTATAAGAGACAGCTCTAAGC | NGS |
| | | CTGCCTCTGTG SEQ ID NO: 152 | |
| FIG. 2b | Deep-Seq-[A]-5-F | TCGTCGGCAGCGTCAGATGTGTATAAGAGACAGCCGGAGAAA | NGS |
| | | AGAGAGTTGCA SEQ ID NO: 153 | |
| FIG. 2b | Deep-Seq-[A]-5-R | GTCTCGTGGGCTCGGAGATGTGTATAAGAGACAGACTACAGA | NGS |
| | | CCGTCACATTAGC SEQ ID NO: 154 | |
| FIG. 2b | Deep-Seq-[A]-6-F | TCGTCGGCAGCGTCAGATGTGTATAAGAGACAGGATACATGG | NGS |
| | | AATGAGGGAAGGG SEQ ID NO: 155 | |
| FIG. 2b | Deep-Seq-[A]-6-R | GTCTCGTGGGCTCGGAGATGTGTATAAGAGACAGCTCTCTCCA | NGS |
| | | TCCTCTGAAATGTTC SEQ ID NO: 156 | |
| FIG. 2b | Deep-Seq-[A]-7-F | TCGTCGGCAGCGTCAGATGTGTATAAGAGACAGTGAGAGGCA | NGS |
| | | AAGATGCTTTGA SEQ ID NO: 157 | |
| FIG. 2b | Deep-Seq-[A]-7-R | GTCTCGTGGGCTCGGAGATGTGTATAAGAGACAGTTCTTTCTA | NGS |
| | | CTTTTTAAATGTGGCCA SEQ ID NO: 158 | |
| FIG. 2b | Deep-Seq-[A]-8-F | TCGTCGGCAGCGTCAGATGTGTATAAGAGACAGCCCACATGA | NGS |
| | | TCAAATGATAGGAAATG SEQ ID NO: 159 | |
| FIG. 2b | Deep-Seq-[A]-8-R | GTCTCGTGGGCTCGGAGATGTGTATAAGAGACAGGCTCCAGC | NGS |
| | | CGATATTTCAGA SEQ ID NO: 160 | |
| FIG. 2b | Deep-Seq-[A]-9-F | TCGTCGGCAGCGTCAGATGTGTATAAGAGACAGTCCAGGCAA | NGS |
| | | GTATCTTGTTCAG SEQ ID NO: 161 | |
| FIG. 2b | Deep-Seq-[A]-9-R | GTCTCGTGGGCTCGGAGATGTGTATAAGAGACAGCTAGCTGT | NGS |
| | | GTAATTTCCAGATATGC SEQ ID NO: 162 | |
| FIG. 2b | Deep-Seq-[A]-10-F | TCGTCGGCAGCGTCAGATGTGTATAAGAGACAGTTTTTTGTAG | NGS |
| | | AGACAGGGTCTCG SEQ ID NO: 163 | |
| FIG. 2b | Deep-Seq-[A]-10-R | GTCTCGTGGGCTCGGAGATGTGTATAAGAGACAGGAGCAAGT | NGS |
| | | TGTACCTGTCTAGC SEQ ID NO: 164 | |
| FIG. 2b | Deep-Seq-[A]-11-F | TCGTCGGCAGCGTCAGATGTGTATAAGAGACAGAAGGCTTGG | NGS |
| | | ATAGAGGGCA SEQ ID NO: 165 | |
| FIG. 2b | Deep-Seq-[A]-11-R | GTCTCGTGGGCTCGGAGATGTGTATAAGAGACAGCAGGCAAT | NGS |
| | | GTTTCTCCAACAGA SEQ ID NO: 166 | |
| FIG. 2b | Deep-Seq-[A]-12-F | TCGTCGGCAGCGTCAGATGTGTATAAGAGACAGGTTGAAGGT | NGS |
| | | ACTCAGATGCTGAG SEQ ID NO: 167 | |
| FIG. 2b | Deep-Seq-[A]-12-R | GTCTCGTGGGCTCGGAGATGTGTATAAGAGACAGATAGCTGT | NGS |
| | | ATCTTACCGCATGTT SEQ ID NO: 168 | |
| FIG. 2c | Site 1 (TTTA)-F | TCGTCGGCAGCGTCAGATGTGTATAAGAGACAGTGAAGTAGC | NGS |
| | | ATTTCTACCCTGTT SEQ ID NO: 169 | |
| FIG. 2c | Site 1 (TTTA)-R | GTCTCGTGGGCTCGGAGATGTGTATAAGAGACAGATTGGTCA | NGS |
| | | AGATCAGCATGTAAAA SEQ ID NO: 170 | |
| FIG. 2c | Site 2 (TTTC)-F | TCGTCGGCAGCGTCAGATGTGTATAAGAGACAGTGGCTGATC | NGS |
| | | CGTACTAATCCT SEQ ID NO: 171 | |
| FIG. 2c | Site 2 (TTTC)-R | GTCTCGTGGGCTCGGAGATGTGTATAAGAGACAGCCAGCCGT | NGS |
| | | ATGTTTTCTAATAAGCA SEQ ID NO: 172 | |
| FIG. 2d | NGS-Left-1-F | TCGTCGGCAGCGTCAGATGTGTATAAGAGACAGTAGGGGTAC | NGS |
| | | AGACTGCTGGG SEQ ID NO: 173 | |
| FIG. 2d | NGS-Left-1-R | GTCTCGTGGGCTCGGAGATGTGTATAAGAGACAGCTGCAGTG | NGS |
| | | GTCTGGAACCAA SEQ ID NO: 174 | |
| FIG. 2d | NGS-Left-2-F | TCGTCGGCAGCGTCAGATGTGTATAAGAGACAGAGAGGATCG | NGS |
| | | AGGCTCTTCCAGG SEQ ID NO: 175 | |
| FIG. 2d | NGS-Left-2-R | GTCTCGTGGGCTCGGAGATGTGTATAAGAGACAGACAACCAG | NGS |
| | | AGGGGGTAACGC SEQ ID NO: 176 | |
| FIG. 2d | NGS-Left-3-F | TCGTCGGCAGCGTCAGATGTGTATAAGAGACAGCTGAGTCTG | NGS |
| | | CACAGAGCGGG SEQ ID NO: 177 | |
| FIG. 2d | NGS-Left-3-R | GTCTCGTGGGCTCGGAGATGTGTATAAGAGACAGCACTTAGG | NGS |
| | | CAGACATCCTGTGCT SEQ ID NO: 178 | |
| FIG. 2d | NGS-Left-4-F | TCGTCGGCAGCGTCAGATGTGTATAAGAGACAGGGGATTACA | NGS |
| | | GGCGCGCG SEQ ID NO: 179 | |
| FIG. 2d | NGS-Left-4-R | GTCTCGTGGGCTCGGAGATGTGTATAAGAGACAGACAAGTCA | NGS |
| | | ACCAAACGCTGTC SEQ ID NO: 180 | |
| FIG. 2d | NGS-Left-5-F | TCGTCGGCAGCGTCAGATGTGTATAAGAGACAGAAAGATGGG | NGS |
| | | ATTTTGTCATGTTGC SEQ ID NO: 181 | |
| FIG. 2d | NGS-Left-5-R | GTCTCGTGGGCTCGGAGATGTGTATAAGAGACAGGGCAGGC | NGS |
| | | AATGCTGTCT SEQ ID NO: 182 | |
| FIG. 2d | NGS-Left-6-F | TCGTCGGCAGCGTCAGATGTGTATAAGAGACAGATGTTTCTCG | NGS |
| | | GCAACCTTGG SEQ ID NO: 183 | |
| FIG. 2d | NGS-Left-6-R | GTCTCGTGGGCTCGGAGATGTGTATAAGAGACAGCGGGAAA | NGS |
| | | ACTGTCCTCGGTAT SEQ ID NO: 184 | |
| FIG. 2d | NGS-Left-7-F | TCGTCGGCAGCGTCAGATGTGTATAAGAGACAGCGTGTAGTG | NGS |
| | | TTTTTGTAACTGATCGT SEQ ID NO: 185 | |
| FIG. 2d | NGS-Left-7-R | GTCTCGTGGGCTCGGAGATGTGTATAAGAGACAGTAGGCTGA | NGS |
| | | GGGTCGCTGA SEQ ID NO: 186 | |
| FIG. 2e | NGS-Right-1-F | TCGTCGGCAGCGTCAGATGTGTATAAGAGACAGTGTCGGGGC | NGS |
| | | GCTCATAGT SEQ ID NO: 187 | |
| FIG. 2e | NGS-Right-1-R | GTCTCGTGGGCTCGGAGATGTGTATAAGAGACAGCCCTGGTT | NGS |
| | | AAGCCCCTCTG SEQ ID NO: 188 | |
| FIG. 2e | NGS-Right-2-F | TCGTCGGCAGCGTCAGATGTGTATAAGAGACAGAAGTCAGAC | NGS |
| | | CAGCTCTTTCCC SEQ ID NO: 189 | |
| FIG. 2e | NGS-Right-2-R | GTCTCGTGGGCTCGGAGATGTGTATAAGAGACAGGCCTGTTG | NGS |
| | | TAAGCTCCACC SEQ ID NO: 190 | |
| FIG. 2e | NGS-Right-3-F | TCGTCGGCAGCGTCAGATGTGTATAAGAGACAGCCCTGCAAC | NGS |
| | | TTAGGAAGAAAAGAGA SEQ ID NO: 191 | |
| FIG. 2e | NGS-Right-3-R | GTCTCGTGGGCTCGGAGATGTGTATAAGAGACAGCAGCCTTC | NGS |
| | | ACTTCAGGGTGGT SEQ ID NO: 192 | |
| FIG. 2e | NGS-Right-4-F | TCGTCGGCAGCGTCAGATGTGTATAAGAGACAGTGACTGACG | NGS |
| | | CTGATCGCACAT SEQ ID NO: 193 | |
| FIG. 2e | NGS-Right-4-R | GTCTCGTGGGCTCGGAGATGTGTATAAGAGACAGCCTCCCCA | NGS |
| | | GGTTCAGAGGACA SEQ ID NO: 194 | |
| FIG. 2e | NGS-Right-5-F | TCGTCGGCAGCGTCAGATGTGTATAAGAGACAGTAGGACCAA | NGS |
| | | GGTGCGTCAGG SEQ ID NO: 195 | |
| FIG. 2e | NGS-Right-5-R | GTCTCGTGGGCTCGGAGATGTGTATAAGAGACAGGCCGCCAC | NGS |
| | | CGGCTTTATTC SEQ ID NO: 196 | |
| FIG. 2e | NGS-Right-6-F | TCGTCGGCAGCGTCAGATGTGTATAAGAGACAGTTGGCTTTG | NGS |
| | | CTGGGGCTA SEQ ID NO: 197 | |
| FIG. 2e | NGS-Right-6-R | GTCTCGTGGGCTCGGAGATGTGTATAAGAGACAGCCCATGGG | NGS |
| | | TCTAACATTCACAGAA SEQ ID NO: 198 | |
| FIG. 2e | NGS-Right-7-F | TCGTCGGCAGCGTCAGATGTGTATAAGAGACAGCCTCTAGGG | NGS |
| | | AGGTTTCTGTGA SEQ ID NO: 199 | |
| FIG. 2e | NGS-Right-7-R | GTCTCGTGGGCTCGGAGATGTGTATAAGAGACAGGGGATGCT | NGS |
| | | GAAGCAAAGGGTA SEQ ID NO: 200 | |
| FIG. 2f | a(TTTA)-F | TCGTCGGCAGCGTCAGATGTGTATAAGAGACAGGGTTGATGT | NGS |
| | | GTGTTATTATTTGTAATTAT SEQ ID NO: 201 | |
| FIG. 2f | a(TTTA)-R | GTCTCGTGGGCTCGGAGATGTGTATAAGAGACAGCTAACAAC | NGS |
| | | TTCAACTGGATATCCTTATA SEQ ID NO: 202 | |
| FIG. 2f | b(TTTA)-F | TCGTCGGCAGCGTCAGATGTGTATAAGAGACAGCGCAACAGA | NGS |
| | | AAAAGTATTTAAGCAG SEQ ID NO: 203 | |
| FIG. 2f | b(TTTA)-R | GTCTCGTGGGCTCGGAGATGTGTATAAGAGACAGTTGCTAGA | NGS |
| | | CGCTGAAGACTAATTTT SEQ ID NO: 204 | |
| FIG. 2f | c(TTTC)-F | TCGTCGGCAGCGTCAGATGTGTATAAGAGACAGCTGAGAATA | NGS |
| | | TCTAGCAGCAACAT SEQ ID NO: 205 | |
| FIG. 2f | c(TTTC)-R | GTCTCGTGGGCTCGGAGATGTGTATAAGAGACAGGTCTGTTT | NGS |
| | | GATCTCACCATCTT SEQ ID NO: 206 | |
| FIG. 2f | d(TTTC)-F | TCGTCGGCAGCGTCAGATGTGTATAAGAGACAGCGCAAAGCT | NGS |
| | | TCTTCTTGATCTAAAC SEQ ID NO: 207 | |
| FIG. 2f | d(TTTC)-R | GTCTCGTGGGCTCGGAGATGTGTATAAGAGACAGGTTGACAT | NGS |
| | | TCTCTTTGAAGATATGGT SEQ ID NO: 208 | |
| FIG. 2f | e(TTTG)-F | TCGTCGGCAGCGTCAGATGTGTATAAGAGACAGCCAATATTTT | NGS |
| | | CCATAACTTAAGGTGC SEQ ID NO: 209 | |
| FIG. 2f | e(TTTG)-R | GTCTCGTGGGCTCGGAGATGTGTATAAGAGACAGGCATCTAA | NGS |
| | | CAAAGATACTTACATTTGAA SEQ ID NO: 210 | |
| FIG. 2f | f(TTTG)-F | TCGTCGGCAGCGTCAGATGTGTATAAGAGACAGTATTAACTCT | NGS |
| | | GGGCTGCTGT SEQ ID NO: 211 | |
| FIG. 2f | f(TTTG)-R | GTCTCGTGGGCTCGGAGATGTGTATAAGAGACAGGACATAGG | NGS |
| | | AGCAGAGCTGAAG SEQ ID NO: 212 | |
| FIG. 2f | g(TTTT)-F | TCGTCGGCAGCGTCAGATGTGTATAAGAGACAGGCAGAACCC | NGS |
| | | ACTAATACAAAGGA SEQ ID NO: 213 | |
| FIG. 2f | g(TTTT)-R | GTCTCGTGGGCTCGGAGATGTGTATAAGAGACAGCACAGACA | NGS |
| | | AGCCCTCAGATATATT SEQ ID NO: 214 | |
| FIG. 2f | h(TTTT)-F | TCGTCGGCAGCGTCAGATGTGTATAAGAGACAGTCATTGTTTT | NGS |
| | | TACCAAGGATCCAT SEQ ID NO: 215 | |
| FIG. 2f | h(TTTT)-R | GTCTCGTGGGCTCGGAGATGTGTATAAGAGACAGTAAATCAT | NGS |
| | | AGGCTACAGCTGAAA SEQ ID NO: 216 | |
| FIG. 2g | EUR-3 C2-F | TCGTCGGCAGCGTCAGATGTGTATAAGAGACAGTGGCTGATC | NGS |
| | | CGTACTAATCCT SEQ ID NO: 217 | |
| FIG. 2g | EUR-3 C2-R | GTCTCGTGGGCTCGGAGATGTGTATAAGAGACAGCCAGCCGT | NGS |
| | | ATGTTTTCTAATAAGCA SEQ ID NO: 218 | |
| FIG. 3c, | Matched site 2-F | TCGTCGGCAGCGTCAGATGTGTATAAGAGACAGAGCTGGCCT | NGS |
| 4d | | CTAAGACCCCCTT SEQ ID NO: 219 | |
| FIG. 3c, | Matched site 2-R | GTCTCGTGGGCTCGGAGATGTGTATAAGAGACAGCAGCTCTG | NGS |
| 4d | | TCTCTCACCTGGCG SEQ ID NO: 220 | |
| FIG. 3c, | Matched site 3-F | TCGTCGGCAGCGTCAGATGTGTATAAGAGACAGGGAGAGGA | NGS |
| 4d | | TTTGGCTGGTAAC SEQ ID NO: 221 | |
| FIG. 3c, | Matched site 3-R | GTCTCGTGGGCTCGGAGATGTGTATAAGAGACAGCCCTACCT | NGS |
| 4d | | GAGGTCACAAGG SEQ ID NO: 222 | |
| FIG. 3c, | Matched site 9-F | TCGTCGGCAGCGTCAGATGTGTATAAGAGACAGGGCTGTGAT | NGS |
| 4d | | GTGCCTTATGGAGA SEQ ID NO: 223 | |
| FIG. 3c, | Matched site 9-R | GTCTCGTGGGCTCGGAGATGTGTATAAGAGACAGACAGAATA | NGS |
| 4d | | CTGAACCCTTGCTGCCA SEQ ID NO: 224 | |
| FIG. 3c, | Matched site 15-F | TCGTCGGCAGCGTCAGATGTGTATAAGAGACAGTCTTGAGAG | NGS |
| 4d | | AGCATTGGTAGTTTG SEQ ID NO: 225 | |
| FIG. 3c, | Matched site 15-R | GTCTCGTGGGCTCGGAGATGTGTATAAGAGACAGCTCTCATCT | NGS |
| 4d | | TAATCCCTCACAATCC SE226Q ID NO: 226 | |
| FIG. 3c, | Matched site 20-F | TCGTCGGCAGCGTCAGATGTGTATAAGAGACAGGCACGATTC | NGS |
| 4d | | TCAGCCTTCAAG SEQ ID NO: 227 | |
| FIG. 3c, | Matched site 20-R | GTCTCGTGGGCTCGGAGATGTGTATAAGAGACAGCCCGGCCA | NGS |
| 4d | | AACCTTTTTGG SEQ ID NO: 228 | |
| FIG. 4c, | GAPDH-LHA | /5BIOSG/C*A*G*T*T*GCCATGTAGACCCCTTGAAGAGGGGA | Donor generation |
| FIG. 15A- | | GGGGCCTAGGGAGCCGCACTTATATATGTTCACTTCCCGGAGC | |
| 15C, 16 | | SEQ ID NO: 229 | |
| FIG. 4c, | GAPDH-RHA | /5BIOSG/G*A*C*A*A*GTAACTGGTTGAGCACAGGGTACTTT | Donor generation |
| FIG. 15A- | | ATTGATGGTACATGACAAGACCACACTGGACTAGTGATCT | |
| 15C, 16 | | SEQ ID NO: 230 | |
| FIG. 4c | ACTB-LHA | /5BIOSG/G*A*G*G*A*CTTTGATTGCACATTGTTGTTTTTTT | Donor generation |
| | | AATAGTCATTCCAAATATGTTATATATGTTCACTTCCCGGAGC | |
| | | SEQ ID NO: 231 | |
| FIG. 4c | ACTB-RHA | /5BIOSG/A*A*G*T*G*GGGTGGCTTTTAGGATGGCAAGGGA | Donor generation |
| | | CTTCCTGTAACAACGCATCTACCACACTGGACTAGTGATCT | |
| | | SEQ ID NO: 232 | |
| FIG. 4c | HPRT1-LHA | /5BIOSG/G*T*A*A*T*GTTGACTGTATTTTCCAACTTGTTCA | Donor generation |
| | | AATTATTACCAGTGAATCTTTATATATGTTCACTTCCCGGAGC | |
| | | SEQ ID NO: 233 | |
| FIG. 4c | HPRT1-RHA | /5BIOSG/T*A*A*A*T*TTTTGGGAATTTATTGATTTGCATTT | Donor generation |
| | | AAAAGGGAACTGCTGACAAACCACACTGGACTAGTGATCT SEQ | |
| | | ID NO: 234 | |
| FIG. 4c | LMNB1-LHA | /5BIOSG/A*A*T*A*T*ATTGAACTITTGTACTGAATTTTTTT | Donor generation |
| | | GTAATAAGCAATCAAGGTTTTATATATGTTCACTTCCCGGAGC | |
| | | SEQ ID NO: 235 | |
| FIG. 4c | LMNB1-RHA | /5BIOSG/A*T*T*A*G*GTTAATATTGCCTTCTTACAAAATTT | Donor generation |
| | | CTATTTTAAAAAAAATTATACCACACTGGACTAGTGATCT SEQ | |
| | | ID NO: 236 | |
| FIG. 4c, | UBC-LHA | /5BIOSG/G*G*G*G*G*TGTCTAAGTTTCCCCTTTTAAGGTTT | Donor generation |
| FIG. 15A- | | CAACAAATTTCATTGCACTTTATATATGTTCACTTCCCGGAGC | |
| 15C, 16 | | SEQ ID NO: 237 | |
| FIG. 4c, | UBC-RHA | /5BIOSG/A*A*A*A*A*AAAAAAAAACACCAATTGGGAATGCA | Donor generation |
| FIG. 15A- | | ACAACTTTATTGAAAGGAAACCACACTGGACTAGTGATCT SEQ | |
| 15C, 16 | | ID NO: 238 | |
| FIG. 4c, | B2M-LHA | /5BIOSG/C*A*T*A*C*TCTGCTTAGAATTTGGGGGAAAATTT | Donor generation |
| FIG. 15A- | | AGAAATATAATTGACAGGATTATATATGTTCACTTCCCGGAGC | |
| 15C, 16 | | SEQ ID NO: 239 | |
| FIG. 4c, | B2M-RHA | /5BIOSG/T*G*A*A*T*CTTATATGACAAAATGTTTCATTCAT | Donor generation |
| FIG. 15A- | | TATAACAAATTTCCAATAAACCACACTGGACTAGTGATCT SEQ | |
| 15C, 16 | | ID NO: 240 | |
| FIG. 4d | Matched site C-F | TCGTCGGCAGCGTCAGATGTGTATAAGAGACAGAAGGGAGA | NGS |
| | | GGTCAAGGTTGG SEQ ID NO: 241 | |
| FIG. 4d | Matched site C-R | GTCTCGTGGGCTCGGAGATGTGTATAAGAGACAGTGCAATTG | NGS |
| | | CCTTAAACACACCT SEQ ID NO: 242 | |
| FIG. 4d | Matched site F-F | TCGTCGGCAGCGTCAGATGTGTATAAGAGACAGGTATTTCCTC | NGS |
| | | AGGGAAGGACGAAG SEQ ID NO: 243 | |
| FIG. 4d | Matched site F-R | GTCTCGTGGGCTCGGAGATGTGTATAAGAGACAGAGTAGCTG | NGS |
| | | GGACCACAGGA SEQ ID NO: 244 | |
| FIG. 4d | Matched site P-F | TCGTCGGCAGCGTCAGATGTGTATAAGAGACAGGGCCCAGGG | NGS |
| | | AGCTCTTAATAC SEQ ID NO: 245 | |
| FIG. 4d | Matched site P-R | GTCTCGTGGGCTCGGAGATGTGTATAAGAGACAGTTCCTGCT | NGS |
| | | GTAACGCTTAGGC SEQ ID NO: 246 | |
| FIG. 9C | T to A-F | CCTATTGGTTTAAAAATGAGCTGATTTAAC | Site-directed |
| | | SEQ ID NO: 247 | mutagenesis |
| FIG. 9C | T to A-R | CCGAAATCGGCAAAATCC SEQ ID NO: 248 | Site-directed |
| | | | mutagenesis |
| FIG. 9C | Tto G-F | CCTATTGGTTCAAAAATGAGCTGATTTAAC | Site-directed |
| | | SEQ ID NO: 249 | mutagenesis |
| FIG. 9C | Tto G-R | CCGAAATCGGCAAAATCC SEQ ID NO: 250 | Site-directed |
| | | | mutagenesis |
| FIG. 9C | T to C-F | CCTATTGGTTGAAAAATGAGCTG SEQ ID NO: 251 | Site-directed |
| | | | mutagenesis |
| FIG. 9C | T to C-R | CCGAAATCGGCAAAATCC SEQ ID NO: 252 | Site-directed |
| | | | mutagenesis |
| FIG. 9D | DMNT1-F | CTGCTGAAGCCTCCGAGATG SEQ ID NO: 253 | dsDNA substrate |
| | | | generation |
| FIG. 9D | DMNT1-R | AAGCAGCAGACCTTAGCAGG SEQ ID NO: 254 | dsDNA substrate |
| | | | generation |
| FIG. 10 | GAPDH-F | TGCTTACCTAGTGGAGAC SEQ ID NO: 255 | T7EI |
| FIG. 10 | GAPDH-R | TGGTCACTGCAGAATGAC SEQ ID NO: 256 | T7EI |
| FIG. 10 | HPRT1-F | ACACAGCAATACCCTATC SEQ ID NO: 257 | T7EI |
| FIG. 10 | HPRT1-R | CCAGCCGTATGTTTTCTA SEQ ID NO: 258 | T7EI |

### HDR Mediated Reporter Gene Knock-In

IRES-EGFP containing dsDNA donor was amplified from the plasmid (Table 3) using PrimeSTAR Max polymerase (Takara, R045A) with homology arm-containing primers.

TABLE 3
Sequences of plasmids and gene fragments used in this study

pCYA-P5 (Amp+)
Used in FIG. 1d, FIG. 9A, 9B, 9C, 9E
CTCATGACCAAAATCCCTTAACGTGAGTTACGCGCGCGTCGTTCCACTGAGCGTCAGACCCCGTAGAAAAGATC
AAAGGATCTTCTTGAGATCCTTTTTTTCTGCGCGTAATCTGCTGCTTGCAAACAAAAAAACCACCGCTACCAGCG
GTGGTTTGTTTGCCGGATCAAGAGCTACCAACTCTTTTTCCGAAGGTAACTGGCTTCAGCAGAGCGCAGATACC
AAATACTGTTCTTCTAGTGTAGCCGTAGTTAGCCCACCACTTCAAGAACTCTGTAGCACCGCCTACATACCTCGC
TCTGCTAATCCTGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTGGAGCGAACGA
CCTACACCGAACTGAGATACCTACAGCGTGAGCTATGAGAAAGCGCCACGCTTCCCGAAGGGAGAAAGGCGG
ACAGGTATCCGGTAAGCGGCAGGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGAGTGAGCTGATACCGCTCGCCGCAGC
CGAACGACCGAGCGCAGCGAGTCAGTGAGCGAGGAAGCGGAAGGCGAGAGTAGGGAACTGCCAGGCATCAA
ACTAAGCAGAAGGCCCCTGACGGATGGCCTTTTTGCGTTTCTACAAACTCTTTCTGTGTTGTAAAACGACGGCCA
GTCTTAAGCTCGGGCCCCCTGGGCGGTTCTGATAACGAGTAATCGTTAATCCGCAAATAACGTAAAAACCCGCT
TCGGCGGGTTTTTTTATGGGGGGAGTTTAGGGAAAGAGCATTTGTCAGAATATTTAAGGGCGCCTGTCACTTTG
CTTGATATATGAGAATTATTTAACCTTATAAATGAGAAAAAAGCAACGCACTTTAAATAAGATACGTTGCTTTTT
CGATTGATGAACACCTATAATTAAACTATTCATCTATTATTTATGATTTTTTGTATATACAATATTTCTAGTTTGTT
AAAGAGAATTAAGAAAATAAATCTCGAAAATAATAAAGGGAAAATCAGTTTTTGATATCAAAATTATACATGTC
AACGATAATACAAAATATAATACAAACTATAAGATGTTATCAGTATTTATTATCATTTAGAATAAATTTTGTGTCG
CCCTTAATTGTGAGCGGATAACAATTACGAGCTTCATGCACAGTGGCGTTGACATTGATTATTGACTAGTTATTA
ATAGTAATCAATTACGGGGTCATTAGTTCATAGCCCATATATGGAGTTCCGCGTTACATACCCGGGGCCACCCC
ACGGATCCCCCCGAATGCCTTGGAATTAGAGTACCTGTACGGTGGCGGATCGGGATCCGGAAGCGGAGAGGG
CAGAGGAAGTCTTCTAACATGCGGTGACGTGGAGGAGAATCCCGGCCCTATGGTGAGCAAGGGCGAGGAGCT
GTTCACCGGGGTGGTGCCCATCCTGGTCGAGCTGGACGGCGACGTAAACGGCCACAAGTTCAGCGTGTCCGGC
GAGGGCGAGGGCGATGCCACCTACGGCAAGCTGACCCTGAAGTTCATCTGCACCACCGGCAAGCTGCCCGTGC
CCTGGCCCACCCTCGTGACCACCCTGACCTACGGCGTGCAGTGCTTCAGCCGCTACCCCGACCACATGAAGCAG
CACGACTTCTTCAAGTCCGCCATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAGGGCATCGA
CTTCAAGGAGGACGGCAACATCCTGGGGCACAAGCTGGAGTACAACTACAACAGCCACAACGTCTATATCATG
GCCGACAAGCAGAAGAACGGCATCAAGGTGAACTTCAAGATCCGCCACAACATCGAGGACGGCAGCGTGCAG
CTCGCCGACCACTACCAGCAGAACACCCCCATCGGCGACGGCCCCGTGCTGCTGCCCGACAACCACTACCTGAG
CACCCAGTCCGCCCTGAGCAAAGACCCCAACGAGAAGCGCGATCACATGGTCCTGCTGGAGTTCGTGACCGCC
GCCGGGATCACTCTCGGCATGGACGAGCTGTACAAGTAAGGAATTAGATCACTAGTCCAGTGTGGTGGAATTA
TAACTTCGTATAGCATACATTATACGAAGTTATAATTCTGCAGATATCCAGCACAGTGGCGGCCACCTAGGTCTT
GAAAGGAGTGGGAATTGGCTCCGGTGCCCGTCAGTGGGCAGAGCGCACATCGCCCACAGTCCCCGAGAAGTT
GGGGGGAGGGGTCGGCAATTGATCCGGTGCCTAGAGAAGGTGGCGCGGGGTAAACTGGGAAAGTGATGTCG
TGTACTGGCTCCGCCTTTTTCCCGAGGGTGGTGGAGAACCGTATATAAGTGCAGTAGTCGCCGTTAACGTTCTT
TTTCGCAACGGGTTTGCCGCCAGAACACAGGATGACCGAGTACAAGCCCACGGTGCGCCTCGCCACCCGCGAC
GACGTCCCCCGGGCCGTACGCACCCTCGCCGCCGCGTTCGCCGACTACCCCGCCACGCGCCACACCGTCGACCC
GGACCGCCACATCGAGCGGGTCACCGAGCTGCAAGAACTCTTCCTCACGCGCGTCGGGCTCGACATCGGCAAG
GTGTGGGTCGCGGACGACGGCGCCGCGGTGGCGGTCTGGACCACGCCGGAGAGCGTCGAAGCGGGGGCGGT
GTTCGCCGAGATCGGCCCGCGCATGGCCGAGTTGAGCGGTTCCCGGCTGGCCGCGCAGCAACAGATGGAAGG
CCTCCTGGCGCCGCACCGGCCCAAGGAGCCCGCGTGGTTCCTGGCCACCGTCGGCGTCTCGCCCGACCACCAG
GGCAAGGGTCTGGGCAGCGCCGTCGTGCTCCCCGGAGTGGAGGCGGCCGAGCGCGCCGGGGTGCCCGCCTTC
CTGGAGACCTCCGCGCCCCGCAACCTCCCCTTCTACGAGCGGCTCGGCTTCACCGTCACCGCCGACGTCGAGGT
GCCCGAAGGACCGCGCACCTGGTGCATGACCCGCAAGCCCGGTGCCTGAATGAATAAGGCCGCTCGAATAACT
TCGTATAGCATACATTATACGAAGTTATTCGAGTCTAGAGGGCCCGTTAGATCTACAGCTTCCTGCCAACTTGAC
ACTGCGTGGGGCTAGCAAAATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGAGGAAATTGCATCACAACACT
CAACCCTATCTCGGTCTATTCTTTTGATTTATAAGGGATTTTGCCGATTTCGGCCTATTGGTTAAAAAATGAGCTG
ATTTAACAAAAATTTAACGCGAATTAATTCTGTGGAATGTGTGTCAGTTAGGGTGTGGAAAGTCCCCAGGCTCC
CCAGCAGGCAGAAGTATGCAAAGCATGCATCTCAATTAGTCAGCAACCAGGTGTGGAAAGTCCCCAGGCTCCC
CAGCAGGCAGAAGTATGCAAAGCATGCATCTCAATTAGTCAGCAACCATAGTCCCGCCCCTAACTCCGCCCATC
CCGCCCCTAACTCCGCCCAGTTCCGCCCATTCTCCGCCCCATGGCTGACTAATTTTTTTTATTTATGCAGAGGCCG
AGGCCGCCTCTGCCTCTGAGCTATTCCAGAAGTAGTGAGGAGGCTTTTTTGGAGGCCTAGGCTTTTGCAAAAAG
CTCCCGGGAGCTTGTATATCCATTTTCGGATCTGATCAGCACGTGATGAAAAAGCCTGAACTCACCGCGACGTC
TGTCGAGAAGTTTCTGATCGAAAAGTTCGACAGCGTTTCCGACCTGATGCAGCTCTCGGAGGGCGAAGAATCTC
GTGCTTTCAGCTTCGATGTAGGAGGGCGTGGATATGTCCTGCGGGTAAATAGCTGCGCCGATGGTTTCTACAAA
GATCGTTATGTTTATCGGCACTTTGCATCGGCCGCGCTCCCGATTCCGGAAGTGCTTGACATTGGGGAATTCAG
CGAGAGCCTGACCTATTGCATCTCCCGCCGTGCACAGGGTGTCACGTTGCAAGACCTGCCTGAAACCGAACTGC
CCGCTGTTCTGCAGCCGGTCGCGGAGGCCATGGATGCGATCGCTGCGGCCGATCTTAGCCAGACGAGCGGGTT
CGGCCCATTCGGACCGCAAGGAATCGGTCAATACACTACATGGCGTGATTTCATATGCGCGATTGCTGATCCCC
ATGTGTATCACTGGCAAACTGTGATGGACGACACCGTCAGTGCGTCCGTCGCGCAGGCTCTCGATGAGCTGAT
GCTTTGGGCCGAGGACTGCCCCGAAGTCCGGCACCTCGTGCACGCGGATTTCGGCTCCAACAATGTCCTGACG
GACAATGGCCGCATAACAGCGGTCATTGACTGGAGCGAGGCGATGTTCGGGGATTCCCAATACGAGGTCGCCA
ACATCTTCTTCTGGAGGCCGTGGTTGGCTTGTATGGAGCAGCAGACGCGCTACTTCGAGCGGAGGCATCCGGA
GCTTGCAGGATCGCCGCGGCTCCGGGCGTATATGCTCCGCATTGGTCTTGACCAACTCTATCAGAGCTTGGTTG
ACGGCAATTTCGATGATGCAGCTTGGGCGCAGGGTCGATGCGACGCAATCGTCCGATCCGGAGCCGGGACTGT
CGGGCGTACACAAATCGCCCGCAGAAGCGCGGCCGTCTGGACCGATGGCTGTGTAGAAGTACTCGCCGATAGT
GGAAACCGACGCCCCAGCACTCGTCCGAGGGCAAAGGAATAGCACGTGCTACGAGATTTCGATTCCACCGCCG
CCTTCTATGAAAGGTTGGGCTTCGGAATCGTTTTCCGGGACGCCGGCTGGATGATCCTCCAGCGCGGGGATCTC
ATGCTGGAGTTCTTCGCCCACCCCAACTTGTTTATTGCAGCTTATAATGGTTACAAATAAAGCAATAGCATCACA
AATTTCACAAATAAAGCATTTTTTTCACTGCATTCTAGTTGTGGTTTGTCCAAACTCATCAATGTATCTTATCATGT
CTGTATACCGTCGACCTCTAGCTAGAGCTTGGCGTAATCATGGTCATTACCAATGCTTAATCAGTGAGGCACCTA
TCTCAGCGATCTGTCTATTTCGTTCATCCATAGTTGCCTGACTCCCCGTCGTGTAGATAACTACGATACGGGAGG
GCTTACCATCTGGCCCCAGCGCTGCGATGATACCGCGAGAACCACGCTCACCGGCTCCGGATTTATCAGCAATA
AACCAGCCAGCCGGAAGGGCCGAGCGCAGAAGTGGTCCTGCAACTTTATCCGCCTCCATCCAGTCTATTAATTG
TTGCCGGGAAGCTAGAGTAAGTAGTTCGCCAGTTAATAGTTTGCGCAACGTTGTTGCCATCGCTACAGGCATCG
TGGTGTCACGCTCGTCGTTTGGTATGGCTTCATTCAGCTCCGGTTCCCAACGATCAAGGCGAGTTACATGATCCC
CCATGTTGTGCAAAAAAGCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGATGCTTTTCTGTGACTGGT
GAGTACTCAACCAAGTCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTCGGGGCGAAAACTCTCA
AGGATCTTACCGCTGTTGAGATCCAGTTCGATGTAACCCACTCGTGCACCCAACTGATCTTCAGCATCTTTTACTT
TCACCAGCGTTTCTGGGTGAGCAAAAACAGGAAGGCAAAATGCCGCAAAAAAGGGAATAAGGGCGACACGGA
AATGTTGAATACTCATATTCTTCCTTTTTCAATATTATTGAAGCATTTATCAGGGTTATTGTCTCATGAGCGGATA
CATATTTGAATGTATTTAGAAAAATAAACAAATAGGGGTCAGTGTTACAACCAATTAACCAATTCTGAACATTAT
CGCGAGCCCATTTATACCTGAATATGGCTCATAACACCCCTTG SEQ ID NO: 133
Knock-in donor
Used in FIG. 4c, FIG. 15A-15C, 16
TTATATATGTTCACTTCCCGGAGCGGGATCAATTCCGCCCCCCCCCTAACGTTACTGGCCGAAGCCGCTTGGAAT
AAGGCCGGTGTGCGTTTGTCTATATGTTATTTTCCACCATATTGCCGTCTTTTGGCAATGTGAGGGCCCGGAAAC
CTGGCCCTGTCTTCTTGACGAGCATTCCTAGGGGTCTTTCCCCTCTCGCCAAAGGAATGCAAGGTCTGTTGAATG
TCGTGAAGGAAGCAGTTCCTCTGGAAGCTTCTTGAAGACAAACAACGTCTGTAGCGACCCTTTGCAGGCAGCG
GAACCCCCCACCTGGCGACAGGTGCCTCTGCGGCCAAAAGCCACGTGTATAAGATACACCTGCAAAGGCGGCA
CAACCCCAGTGCCACGTTGTGAGTTGGATAGTTGTGGAAAGAGTCAAATGGCTCTCCTCAAGCGTATTCAACAA
GGGGCTGAAGGATGCCCAGAAGGTACCCCATTGTATGGGATCTGATCTGGGGCCTCGGTGCACATGCTTTACA
TGTGTTTAGTCGAGGTTAAAAAAACGTCTAGGCCCCCCGAACCACGGGGACGTGGTTTTCCTTTGAAAAACACG
ATAATATGGCCACAATGGTGAGCAAGGGCGAGGAGCTGTTCACCGGGGTGGTGCCCATCCTGGTCGAGCTGG
ACGGCGACGTAAACGGCCACAAGTTCAGCGTGTCCGGCGAGGGCGAGGGCGATGCCACCTACGGCAAGCTGA
CCCTGAAGTTCATCTGCACCACCGGCAAGCTGCCCGTGCCCTGGCCCACCCTCGTGACCACCCTGACCTACGGC
GTGCAGTGCTTCAGCCGCTACCCCGACCACATGAAGCAGCACGACTTCTTCAAGTCCGCCATGCCCGAAGGCTA
CGTCCAGGAGCGCACCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACATCCTGGGGCACAAG
CTGGAGTACAACTACAACAGCCACAACGTCTATATCATGGCCGACAAGCAGAAGAACGGCATCAAGGTGAACT
TCAAGATCCGCCACAACATCGAGGACGGCAGCGTGCAGCTCGCCGACCACTACCAGCAGAACACCCCCATCGG
CGACGGCCCCGTGCTGCTGCCCGACAACCACTACCTGAGCACCCAGTCCGCCCTGAGCAAAGACCCCAACGAG
AAGCGCGATCACATGGTCCTGCTGGAGTTCGTGACCGCCGCCGGGATCACTCTCGGCATGGACGAGCTGTACA
AGTAAGGAATTAGATCACTAGTCCAGTGTGGT SEQ ID NO: 134

The 50 bp homology arms are incorporated with primers listed in Table 2. PCR products were initially gel-purified using GeneJET gel extraction kit (Thermofisher, K0691) followed by another round of PCR to scale up the amount of the donor. Lyophilization was used to condense donors to get a concentration around 2 μg/μL. 10 μg of each donor was used in EGFP knock-in nucleofection. 1×10⁶ HEK293T cells were transfected using SF Cell Line Nucleofector Kit with Cas12a RNP using DS-150 program on a Lonza 4D-Nucleofector according to the manufacturer's instruction. All nucleofections were supplemented with 300 pmol Cas12a Electroporation Enhancer, and medium was supplemented with HDR enhancer (IDT, 10007910). Flow cytometry was performed after 4 days to assess the EGFP positive cells rate. Cells were trypsinized and resuspended in ˜500 μL PBS for analysis with BD LSRFortessa Cell Analyzer (BD Biosciences). At least 30,000 events were recorded for each sample. Data were analyzed using the BD FACSDiva software (Version 7.0).

### Multiplex Genome Editing

Multiple crRNAs (total 320 μmol) were used in RNP formation. 1×10⁶ U2OS cells were transfected using SE Cell Line Nucleofector Kit with RNP containing 320 pmol crRNAs (or sgRNAs) and 192 pmol AsCas12a (or SpCas9) Nuclease using DN-100 program on a Lonza 4D-Nucleofector according to manufacturer's instructions. 1×10⁶ HEK293T cells were transfected using SF Cell Line nucleofector Kit with RNP using DS-150 program. All nucleofections were supplemented with 300 pmol Cas12a (or Cas9) Electroporation Enhancer. Genomic DNA was extracted approximately 72 hours after nucleofection using a Quick-DNA Miniprep Kit. Genomic DNAs were stored at −20° C. until use.

### Statistics and Reproducibility

All experiments were performed at least three biologically independent experiments, the results are shown as mean s.e.m. For datasets with two groups comparisons were made between groups by one-tailed Welch's t-tests. GraphPad Prism was used for plotting and graphing.

### Example 2 Z-crRNA Exhibits Increased Binding Affinity Towards Target DNA

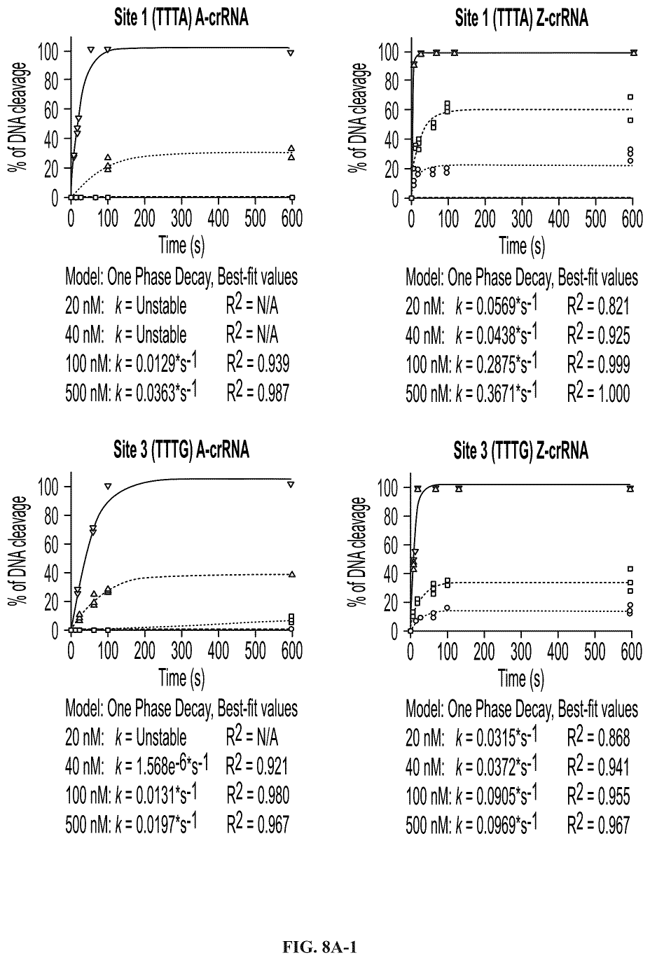

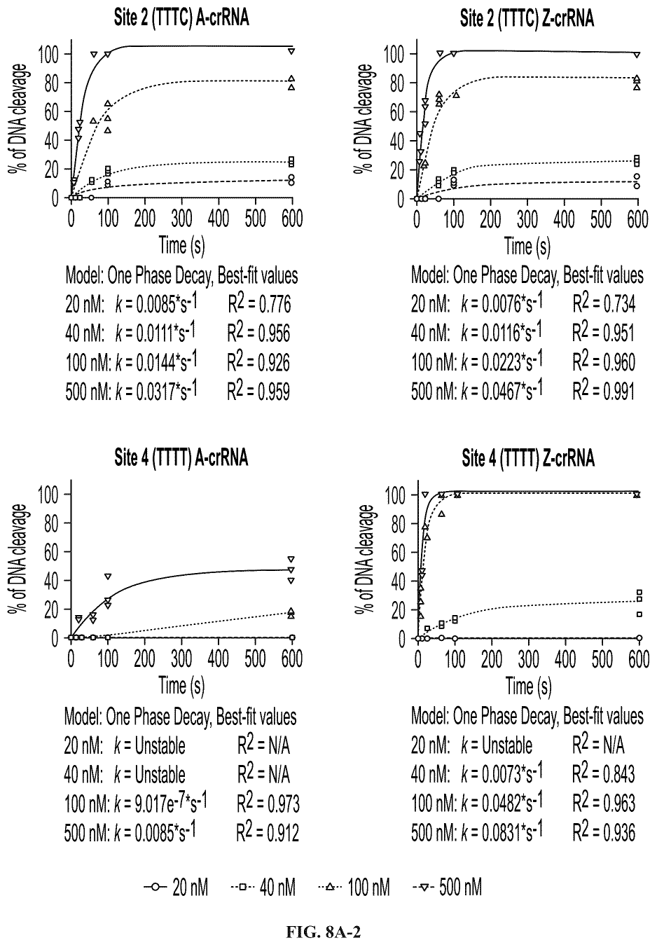

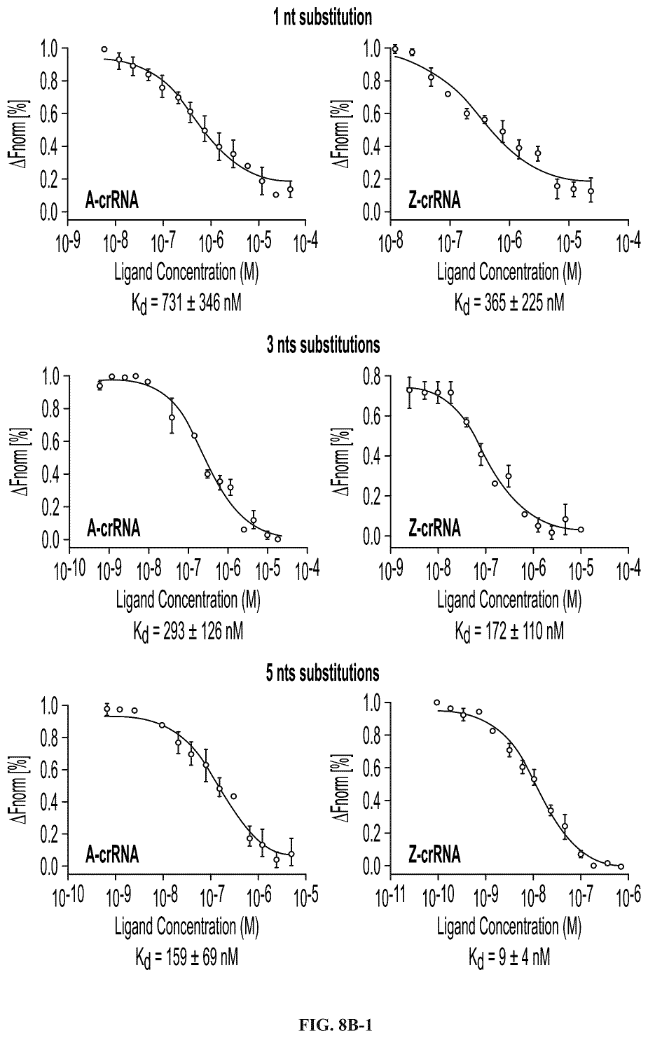

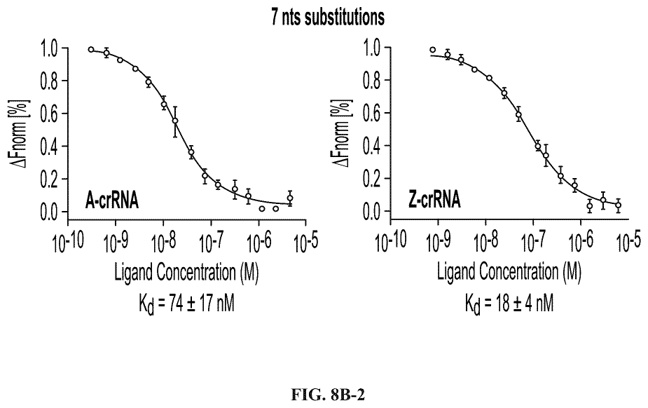

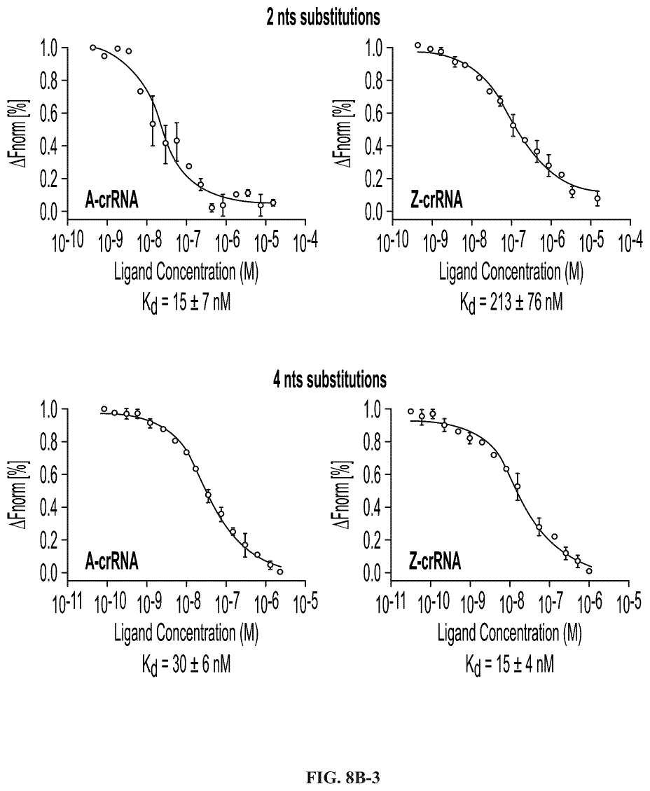

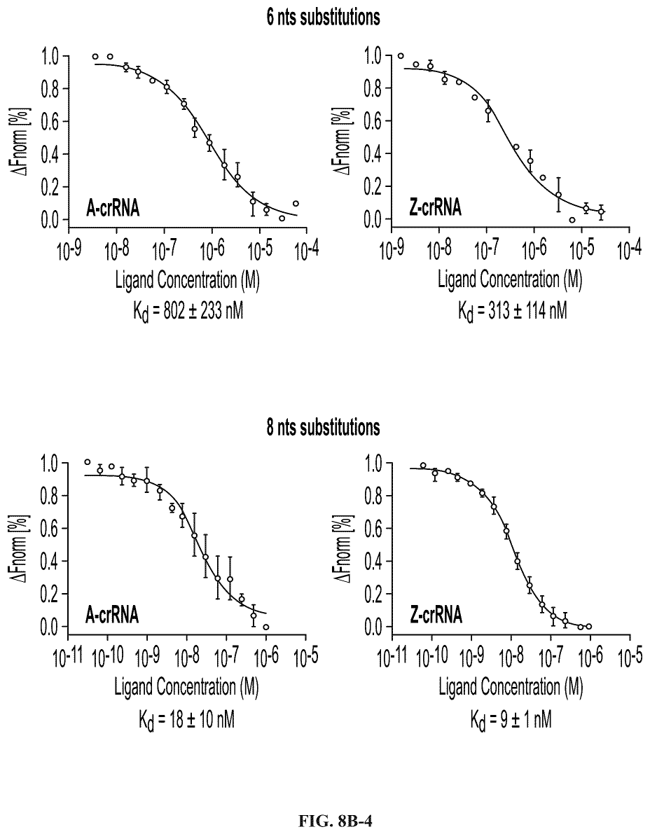

We investigated whether the extra hydrogen bonds from the Z:T pairs would lead to higher binding affinity between crRNA and its target complementary single stranded DNA (ssDNA). We measured the thermostability of the binary complex of in vitro synthesized crRNA (FIG. 1b, FIG. 6) and its corresponding ssDNA. Fluorescence signals from eight binary complexes (which contain incremental numbers of A or Z base in the crRNA spacer region, FIG. 7a) were collected during the annealing step⁴² (FIG. 7b). The findings indicated that the Z-crRNA has an increased propensity to form a binary complex with its complementary ssDNA at high temperatures, which was further augmented with a greater number of Z-base substitutions, i.e. Z-crRNA-ssDNA complex had a higher temperature of annealing (Ta value) than A-crRNA-ssDNA complex (FIG. 1c, FIG. 7a), suggesting that the binding affinity of Z-crRNA-ssDNA is stronger than that of the A-crRNA-ssDNA. Interestingly, the maximum raw fluorescence of Z-crRNA-ssDNA was lower than that of A-crRNA-ssDNA, which is consistent with the gel electrophoresis result (FIG. 6b) that dye molecules hardly intercalated into Z-crRNA. To explore the interaction between the AsCas12a nuclease-crRNA complex and its associated dsDNA substrate, we utilized the microscale thermophoresis (MST) assay. This enabled us to determine the dissociation constant (K_{d}) values for eight target sites, each featuring varying A-to-Z base substitutions. The outcomes demonstrated that introducing base Z within the crRNA could increase the binding affinity between the crRNA and its complementary ssDNA or the ribonucleoprotein (RNP) and dsDNA substrate as the Z-crRNA exhibited a lower K_{d} value at most sites with the exception being the site featuring two substitutions (FIG. 1f, FIG. 8B-1 to 8B-4).

### Z-crRNA Facilitates Cas12a Cleavage Activity

To investigate whether Z-crRNA is able to mediate the cleavage by Cas12a, we performed an in vitro DNA cleavage assay using linearized plasmids and AsCas12a. Indeed, Z-crRNA can mediate the targeted DNA cleavage efficiently and outperform the A-crRNA at three out of four sites at the initial testing, particularly those with TTTT PAM (FIG. 1d, FIG. 9A). The latter is unexpected because the TTTT PAM is known to have a relatively low cleavage efficiency²⁴. To thoroughly assess the efficacy of Z-crRNA in in vitro cleavage assays, we undertook a kinetics study on four sites encompassing various PAM sequences. Our observations indicated a notable enhancement in cleavage efficiency with the use of Z-crRNA, evident in both the plateau and k value. Z-crRNA consistently demonstrated superior performance compared to A-crRNA across all tested sites (FIG. 1e, FIG. 8A-1 to 8A-2). To confirm that zCRISPR-Cas12a can increase the in vitro cleavage efficiency at sites with TTTT PAM, we selected nine additional sites with TTTT PAM for evaluation. Five of them showed improved cleavage efficiency while the rest maintained a similar cleavage efficiency as the A-crRNA (FIG. 9B). Taken together, these results demonstrate that Z-crRNA can be recognized by the AsCas12a nuclease for target DNA cleavage with improved efficiency.

### Z-crRNA Enables Enhanced Cas12a-Mediated Genome Editing

Next, we sought to investigate the Z-crRNA performance in cellulo. We selected eight target sites located on the GAPDH and HPRT1 genes for evaluation. A-crRNA and Z-crRNA were incubated with AsCas12a nuclease to create the RNPs respectively, which were subsequently introduced into HCT116 cells through nucleofection (FIG. 2a). The T7EI assay was used to evaluate the effectiveness of on-target editing, and it was discovered that the use of Z-crRNA resulted in a significant improvement in editing efficiency, with the most improved site experiencing an increase from 17% to 65% (FIG. 10a).

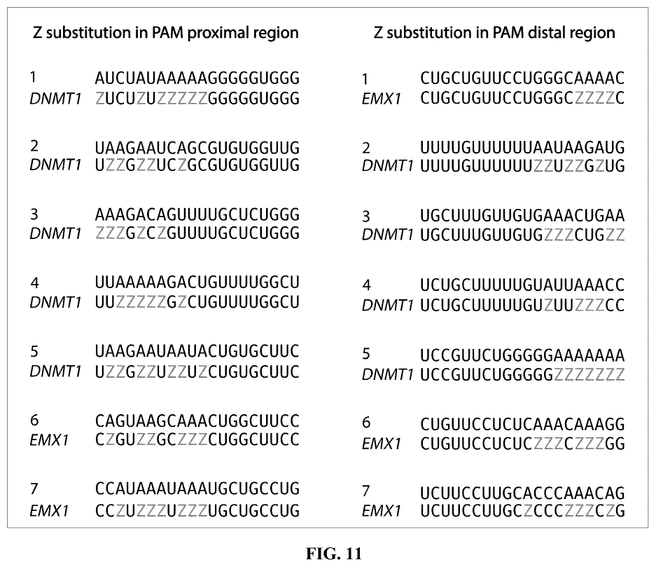

Inspired by these results, we further explored the Z-crRNA design rules for achieving better performance. We investigated whether a higher percentage of the Z base in the crRNA could lead to higher editing efficiency. Thirteen endogenous sites, targeting the EMX1 gene with an incremental number of Z substitutions in the spacer region, were selected to investigate the on-target editing efficiency. The on-target indel frequency was measured by next generation sequencing (NGS), and the results indicate that a larger number of Z base substitutions in the spacer region resulted in better performance (FIG. 2b). Specifically, at the selected sites of this particular gene, when the number of Z substitutions in a crRNA was increased to more than eight out of the 21 nucleotides in the spacer region, Z substitution improved the on-target editing efficiency to up to 93%, compared with nearly zero editing efficiency observed in using A-crRNA. While the utilization of Z-crRNA yielded a substantial improvement in on-target efficiency, the editing efficiency of A-crRNA in this specific gene was unexpectedly low. Furthermore, the enhanced on-target efficiency with Z-crRNA was only evident when the number of substitutions exceeded seven. To prevent any bias in target site selection, we extended our investigation to two additional genes, TPCN2 and RNF2. Notably, the overall editing efficiency on these genes did not exhibit the same degree of limitation in using A-crRNA as observed in the EMX1 gene. Instead, a consistent pattern emerged, indicating that the performance of Z-crRNA is influenced by the number of substitutions. Notably, we observed that introducing only one or two base Z substitutions in these two genes was sufficient to enhance the on-target efficiency (FIG. 10b). In addition, we investigated whether the position of the Z substitution may affect the in cellulo cleavage efficiency of the Cas12a nuclease, given that the PAM proximal region (also known as the seed region) plays a more critical role in target sequence recognition than the PAM distal region⁴³. We selected seven sites where the Z substitutions were located in either PAM proximal or distal region from EMX1 and DNMT1 genes for validation (FIG. 11). On-target indel frequency was assessed at each of the seven sites, and the indel frequency resulting from A-crRNA-mediated editing was subtracted from that observed with Z-crRNA-mediated editing to show the impact of Z substitution. The results are consistent with our hypothesis that Z substitution in the PAM proximal region is more beneficial for editing efficiency improvement than in the PAM distal region (FIG. 2d, 2e).

zCRISPR-Cas12a Shows Improved Editing at Low-Editing-Efficiency Sites

Since the length of crRNA may play an essential role in target DNA recognition, we investigated the impact of the length of the spacer region on the editing efficiency. We truncated the spacer region from 22 nt to 16 nt one by one, starting at the 3′ end of the crRNA. We found that the Z substitution on crRNA efficiently promoted the on-target editing even when the spacer region was as short as 17 nt (FIG. 2c). Based on the aforementioned Z-crRNA design rules, we applied this strategy to 24 previously reported low-editing-efficiency sites²⁴ and performed in cellulo genome editing in both HEK 293T and HCT 116 cell lines. NGS analysis results demonstrate that the zCRISPR-Cas12a was able to elevate the editing efficiency at all characterized low-editing-efficiency sites (FIG. 2f, FIG. 10c). Notably, the largest improvement in editing efficiency relative to the canonical CRISPR-Cas12a was observed at A1 site in HCT116 cell line, which showed an increase from 1.3% to 93.9% (FIG. 2f). While statistically significant improvements were observed across all testing sites, certain sites demonstrated a more modest overall enhancement. To bolster the on-target efficiency in these specific locations, we combined our Z-crRNA strategy with a previously reported engineered variant of AsCas12a, known as AsCas12a Ultra. This approach yielded further improvements on most sites. Notably, the outcome highlighted the compatibility of our Z-crRNA with this enhanced nuclease variant, confirming its upgraded functionality (FIG. 2g, FIG. 10d). Moreover, Z-crRNA could maintain comparable editing efficiency even at RNP concentration eight times lower than what was suggested by the manufacturer (Integrated DNA Technologies, Inc.)²⁴ (FIG. 2h). Taken together, these results indicate that Z-crRNA indeed significantly improved the Cas12a-based genome editing efficiency in mammalian cells.

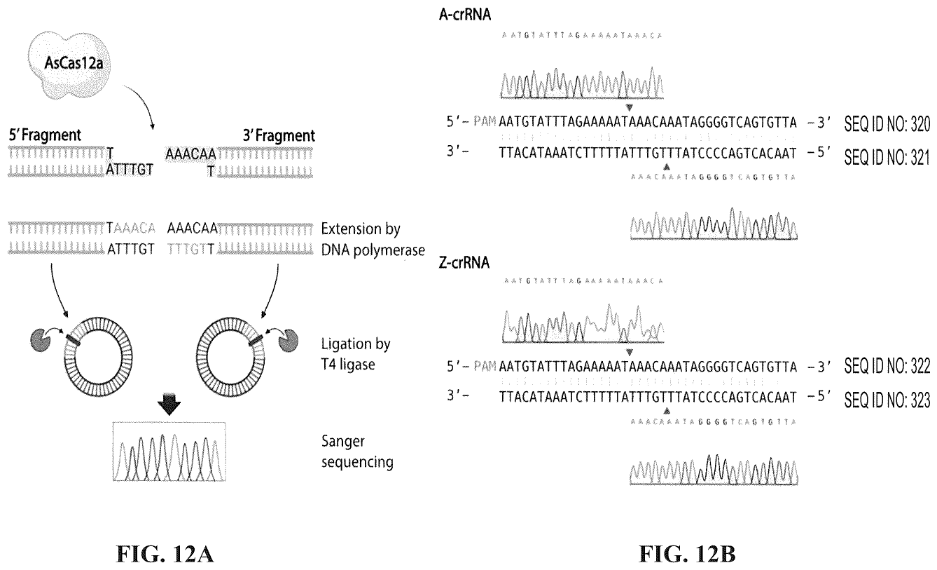

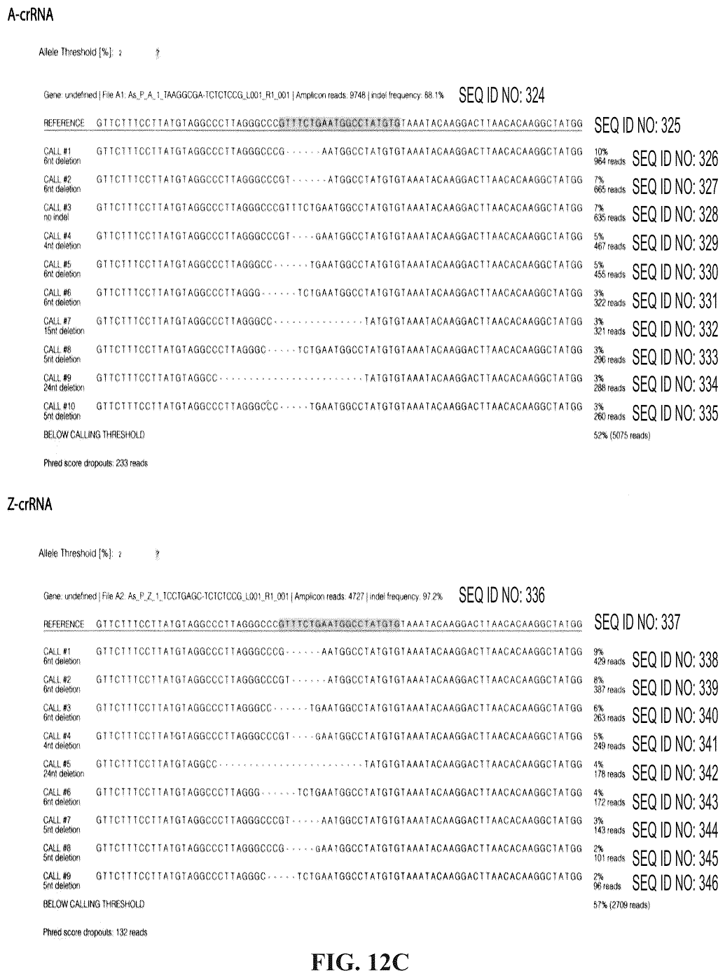

To ensure consistent cleavage position patterns when employing Z-crRNA for on-target cleavage, we conducted in vitro cleavage assays using both A-Cas12a RNP and Z-Cas12a RNP on a dsDNA substrate. The resulting fragments were subcloned into a plasmid respectively for subsequent Sanger sequencing. Remarkably, the results demonstrated that the cleavage position pattern remained unaltered when utilizing Z-crRNA, mirroring the same pattern observed with A-crRNA (FIG. 12a, 12b). Additionally, we extended our investigation to the indel profile in a cell-based assay using Z-crRNA. Through NGS analysis, we determined that there were no notable distinctions between A-crRNA and Z-crRNA, affirming that the incorporation of Z-crRNA does not affect the indel profile in mammalian cells (FIG. 12c).

### Utilizing Z-crRNA Preserves the Low Off-Target Merit of Cas12a

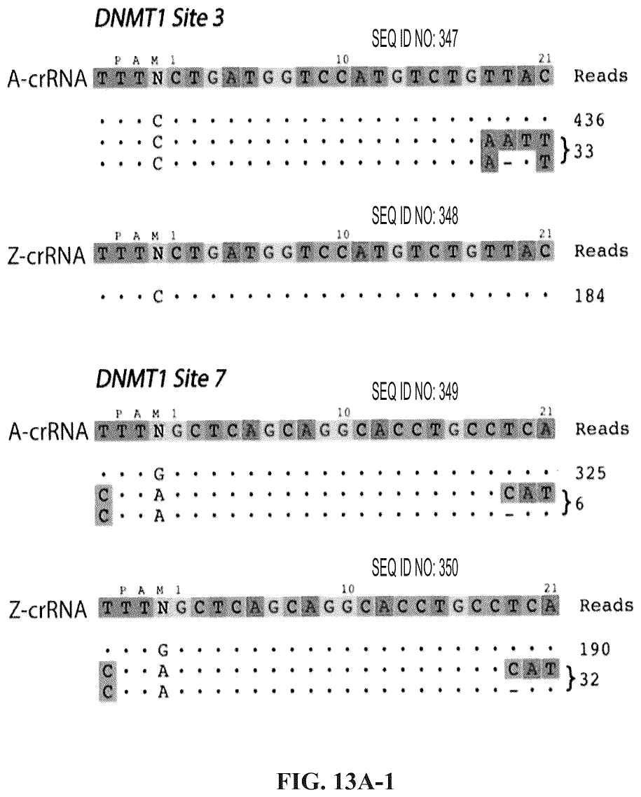

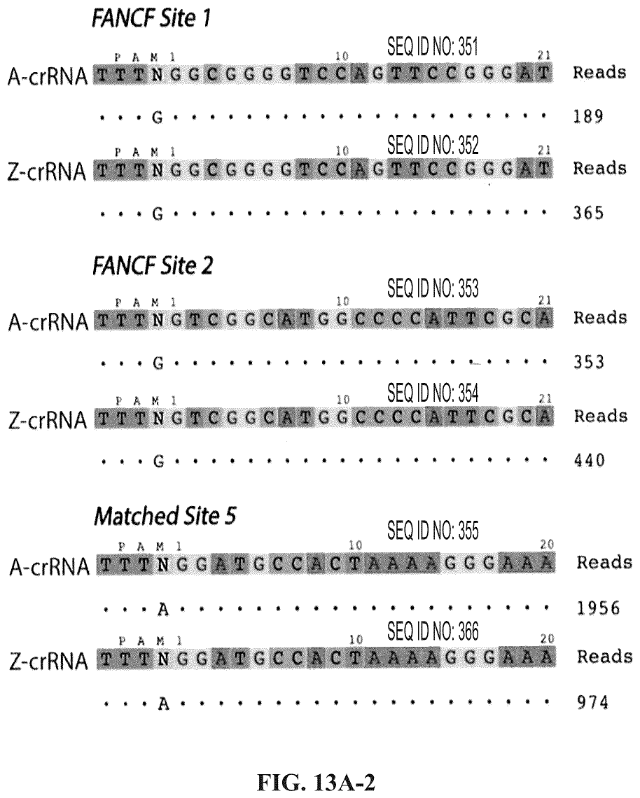

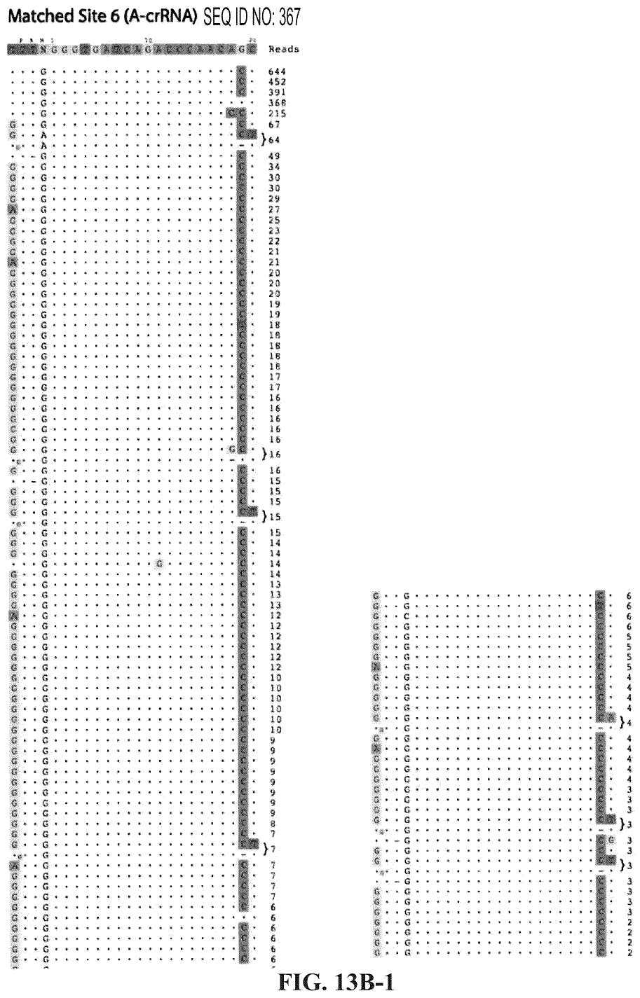

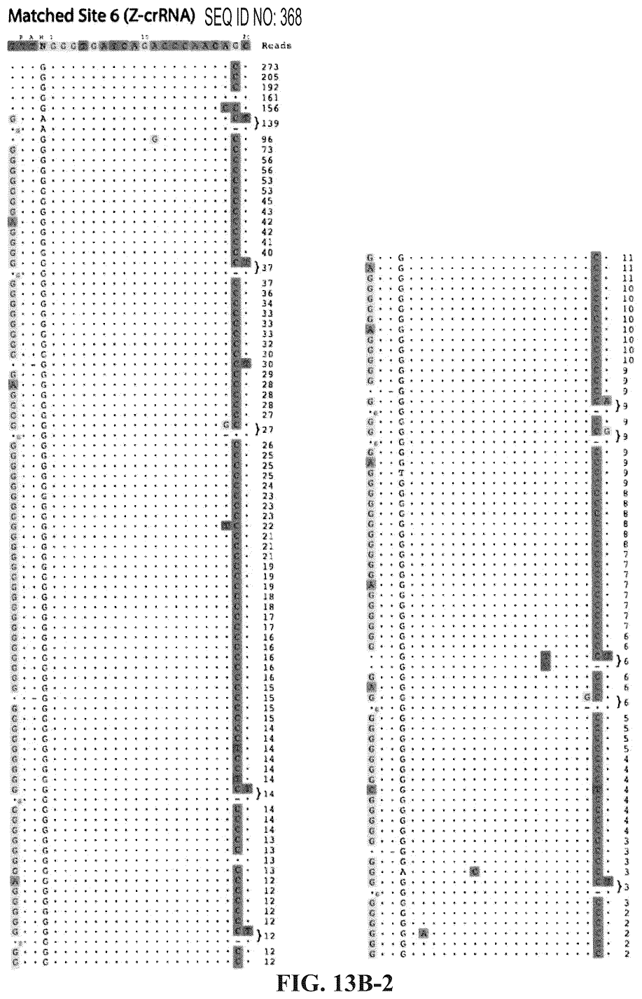

To investigate whether the increased on-target editing efficiency by using Z-crRNA may sacrifice Cas12a intrinsically low off-target property, we harnessed GUIDE-Seq^{12, 44} to assess the off-target cleavage frequency of the A-crRNA and Z-crRNA mediated AsCas12a systems. We selected four different previously reported target sites in DNMT1 and FANCF genes as well as two matched sites¹² as editing targets. The matched sites are the overlapping sequences in the spacer region for both Cas12a and Cas9 nucleases (FIG. 3a). The results confirmed that there was no extra off-target cleavage caused by Z substitutions in crRNA (FIG. 3b, FIG. 13), and the off-target sites detected were consistent with previously reported data¹². Given the high specificity of the Cas12a in mammalian cells, detecting off-target cleavage events for most tested crRNAs can be challenging⁴⁵. We employed the matched site 6, a site previously reported to have numerous off-target editing events, as our positive control to validate the quality of GUIDE-Seq. The effectiveness of the experimental performance was confirmed by the detection of a considerable number of off-target sites throughout the genome, as revealed by our results (FIG. 3b, FIG. 13).

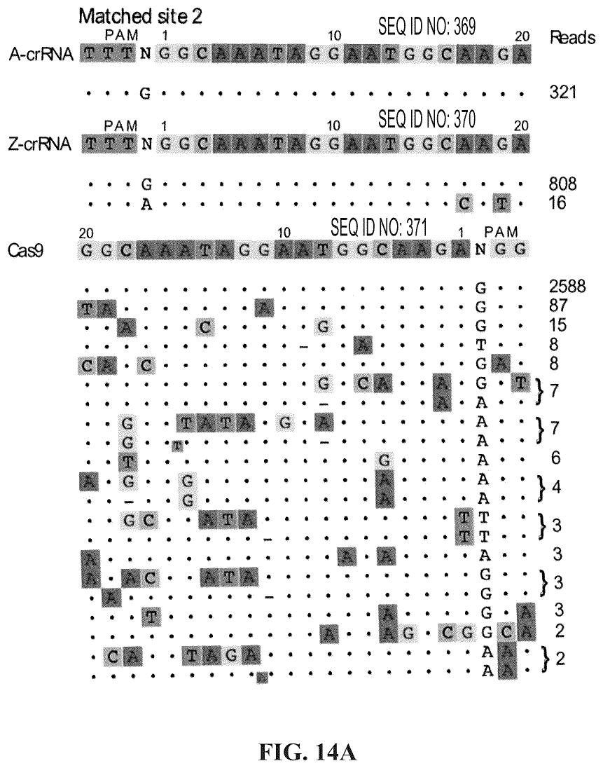

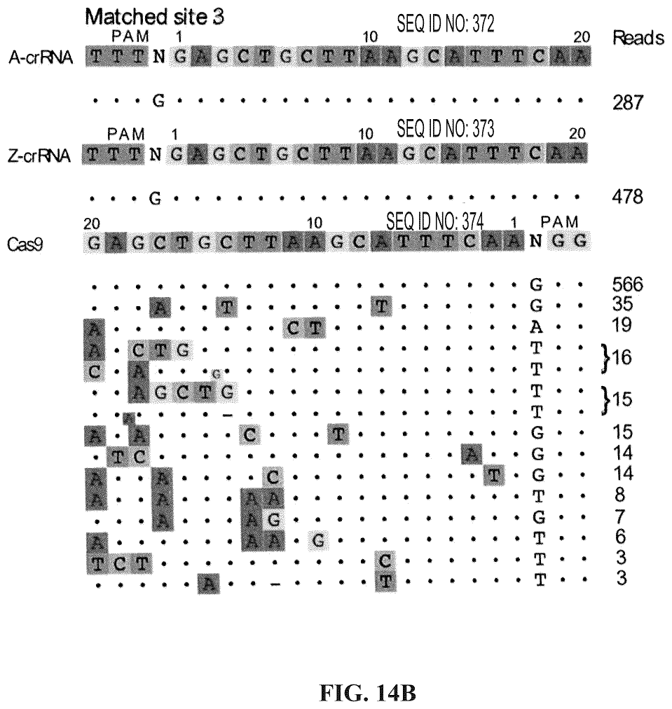

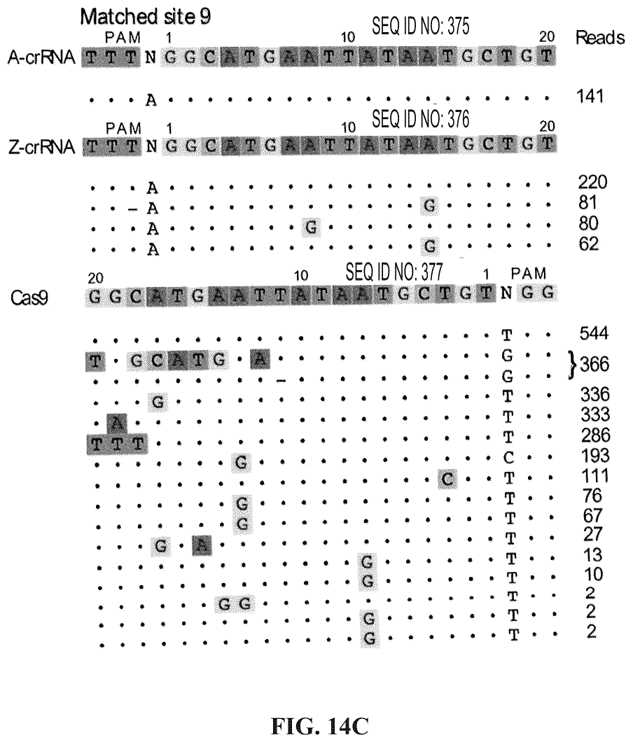

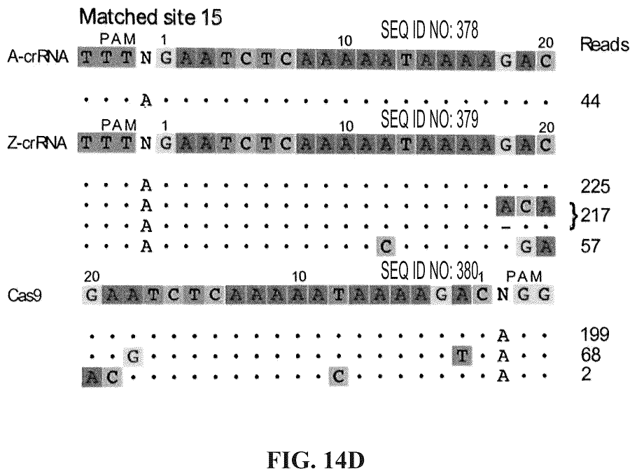

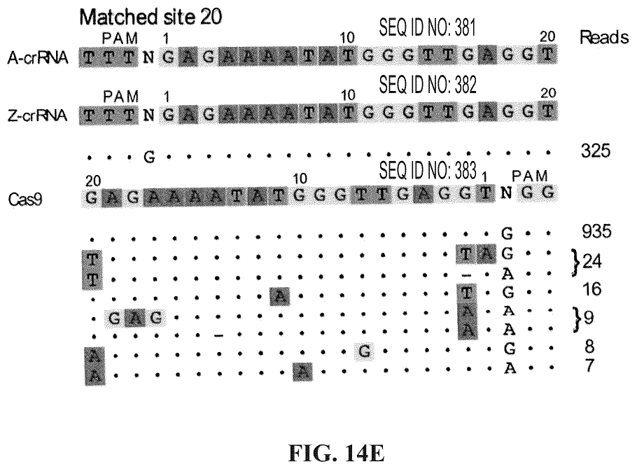

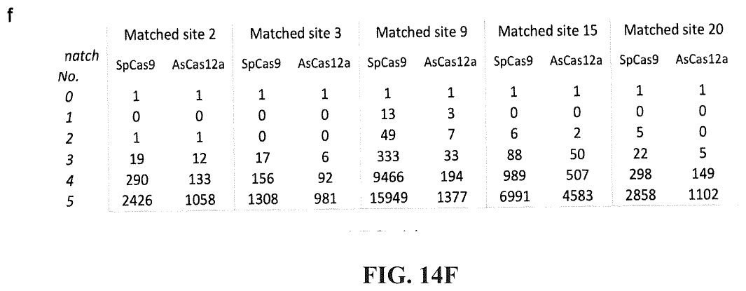

In addition, we performed a side-by-side comparison between zCRISPR-Cas12a and CRISPR-Cas9. Five matched sites, which showed low to nearly zero editing efficiency with AsCas12a in a previous study¹², were selected to compare both on-target editing efficiency and off-target effect of AsCas12a and SpCas9. AsCas12a-A-crRNA RNP, AsCas12a-Z-crRNA RNP, and SpCas9-sgRNA RNP were individually transfected into U2OS cells. The on-target editing efficiency and off-target effect were analyzed by NGS and GUIDE-Seq, respectively. As shown in FIG. 3c, the overall indel frequency of AsCas12a was boosted by using Z-crRNA from extremely low to up to 95%, which was comparable to that of SpCas9. Then, we compared the off-target effect between AsCas12a and SpCas9. To remove any bias resulting from site selection, we initially assessed the number of predicted off-target sites for both Cas9 and Cas12a using Cas-OFFinder. The prediction results confirmed that both Cas9 and Cas12a exhibited a comparable number of off-target sites on those five matched sites (FIG. 14f). As anticipated, the Z substitution in crRNA retained Cas12a's low off-target effect in the genome (FIG. 3d, FIG. 14a-e). Although a few off-target sites have been identified by using Z-crRNA, they are rather limited in comparison to the off-target sites associated with Cas9. Noticeably, some identified off-target sites of Z-crRNA mediated editing from matched site 2 and 15 are the sites with the mismatches at the PAM distal region. Based on our previous data (FIG. 2c, FIG. 14a, d), we found that a 17 nt long spacer region in Z-crRNA was adequate to achieve efficient editing in the mammalian cell genome. Consequently, zCRISPR-Cas12a's inability to differentiate such off-target sites is understandable. Moreover, the reason why A-crRNA did not result in off-target effect could be due to its intrinsically low cleavage activity with those A-crRNAs. In contrast, SpCas9 caused numerous unintended edits across the whole genome with all selected sites (FIG. 3d, FIG. 14). This result suggests that Cas9 editing is more prone to target multiple off-target sites in the genome compared to Cas12a, leading to a decrease in editing specificity. Overall, the data indicate that zCRISPR-Cas12a has the potential to significantly increase the efficiency of genome editing on target sites, and the likelihood of off-target effects may vary based on the unique sequence characteristics of the crRNA.

### Evaluation of the Z-crRNA Strategy Efficacy Across Various Cell Types and Cas12a Orthologs

Next, we investigated the performance of zCRISPR-Cas12a in primary cells using human Mesenchymal Stem Cells (hMSCs). The three sites MS-2, MS-3, and MS-15 (refer to the side-by-side comparison experiment) were selected for the validation. As primary cells are known to be more challenging to transfect, the editing efficiency is generally not as high as observed in the U2OS cell line. Nevertheless, we found that using Z-crRNA still significantly enhanced the editing efficiency in hMSCs (FIG. 3e), and the trend observed was consistent with the efficiency in U2OS cells (FIG. 3c). Additionally, we conducted cell viability analysis on four types of cells used in this study. The results demonstrated that Z-crRNA exhibited either comparable or lower cytotoxicity when compared to A-crRNA (FIG. 3f).

Our findings with AsCas12a indicate a substantial enhancement in on-target efficiency with Z-crRNA. To evaluate the broad applicability of the Z-crRNA strategy, we investigated other Cas12a orthologs known for their mammalian cell genome editing activity^{46, 47}. These orthologs include Francisella novicida U112 (FnCas12a), Thiomicrospira sp. XS5 (TsCas12a), Moraxella bovoculi AAX08_00205 (Mb2Cas12a), Moraxella bovoculi AAX11_00205 (Mb3Cas12a), Butyrivibrio sp. NC3005 (BsCas12a), Helcococcus kunzii ATCC 51366 (HkCas12a), Pseudobutyrivibrio xylanivorans DSM 10317 (PxCas12a), Eubacterium rectale (ErCas12a), and Lachnospiraceae bacterium ND2006 (LbCas12a). In this extensive evaluation, ErCas12a and Mb2Cas12a displayed large improvements of the on-target editing efficiency when using Z-crRNA, matching the efficacy observed with AsCas12a. TsCas12a, Mb3Cas12a, HkCas12a, and PxCas12a also exhibited statistically significant improvements by applying Z-crRNA (FIG. 3g). The relatively lower editing efficiency observed with TsCas12a, HkCas12a, and BsCas12a may be attributed to their limited enzymatic activity, which has been reported in previous in vitro cleavage activity studies⁴⁸. In the case of LbCas12a, which exhibited reduced editing efficiency with Z-crRNA, an examination of its crRNA structure revealed two consecutive “A” residues in the loop structure. Our speculation is that substituting Z in this loop may influence the secondary structure of crRNA, consequently affecting the editing efficiency. Since LbCas12a is compatible with various crRNA direct repeat sequences⁴⁸, further investigation can be conducted using different direct repeat sequences to prevent disruption of the loop structure. To investigate the applicability of Z-crRNA to other CRISPR tools, we employed the Z-crRNA strategy with Cas9 for genome editing. Due to potential impacts on secondary structure from base Z substitutions in tracrRNA, we annealed Z-crRNA and A-tracrRNA to create a chimeric gRNA. This gRNA was then assembled with Cas9 protein for genome editing on EMX2, FANCF3, and RUNX1 genes in HEK293T cells. The results revealed that the use of Z-sgRNA led to a decrease in Cas9 editing efficiency (FIG. 10e). Overall, six out of the nine tested Cas12a orthologs demonstrated enhanced on-target editing efficiency when employing Z-crRNA.

### Z-crRNA Enables Enhanced HDR-Mediated Gene Integration

Since Cas12a triggers DNA cleavage at 3′ end of the spacer, the cleavage site is far away from the PAM while the original target sequence is maintained. This unique feature is beneficial to an extra opportunity to cut at the target site after indel formation, thus increasing the chance of homology-directed repair (HDR) mediated gene integration. To explore if Z-crRNA could also be harnessed to enhance HDR mediated knock-in efficiency, we used a reporter system to measure the knock-in efficiency mediated by Cas12a. The donor was designed to contain an upstream internal ribosomal entry site (IRES) and the EGFP gene flanked by 50-base-pair homology arms (HAs)⁴⁹ (FIG. 4c). In addition, the linear donor was modified with 5′-biotinylation and phosphorothioate bonds⁵⁰ (FIG. 4d). We separately co-electroporated A-crRNA- or Z-crRNA-Cas12a RNP with a double-stranded DNA donor into HEK 293T cells to integrate the gene fragment between the last exon and the 3′ untranslated region (UTR) of six housekeeping genes. Knock-in efficiency was measured 4 days post nucleofection by flow cytometry, and the percentage of EGFP positive cell population was used to determine the integration rate (FIG. 4a). The HDR mediated gene knock-in efficiency was enhanced by up to 7.21-fold by using Z-crRNA for four out of six targets, including GAPDH, ACTB, HPRT1, and B2M (FIG. 4e, FIG. 15A-15C). However, the overall improvement of the integration rate of genes HPRT1, LMNB1, UBC, and B2M is limited. We speculated that the previously observed low knock-in efficiency might be attributed to variations in the expression levels of individual housekeeping genes. Therefore, we assessed the on-target efficiency across the mentioned six sites by using both CRISPR-Cas12a and zCRISPR-Cas12a. The NGS data unveiled a substantial enhancement in on-target efficiency with Z-crRNA, particularly for the sites that exhibited modest improvements in the reporter experiments (FIG. 4b). To overcome the constraints of the reporter system, we evaluated the efficiency of homology-directed repair (HDR)-mediated knock-in by utilizing single-stranded oligo DNA (ssODN) donors²⁴. By integrating an EcoRI restriction site in the cutting site, we were able to assess the knock-in efficiency through enzyme digestion check followed by NGS analysis (FIG. 4a, 4f). The results revealed a significant overall enhancement in integration efficiency, with the most remarkable improvement observed at the UBC site, where the knock-in efficiency surged from 16% to 86% (FIG. 4g). Collectively, these results confirm that the zCRISPR-Cas12a can significantly improve HDR mediated knock-in efficiency compared to CRISPR-Cas12a in mammalian cells.

### The Use of Z-crRNA Enhanced the Capability for Multiplex Genome Editing

Simultaneous perturbation of multiple genes can aid in uncovering and controlling the gene interactions and networks that drive cellular functions¹⁵. However, current genome engineering technologies face difficulties in executing simultaneous multiplex perturbations, both in terms of the number and kind of modifications⁵¹. To investigate whether the Z-crRNA can facilitate multiplex genome editing in mammalian cells, we tested the multiplexing capacity of zCRISPR-Cas12a by targeting up to eight endogenous sites simultaneously. We assessed multiplex gene editing in both U2OS and HEK293T cells, employing the RNP delivery method for both Cas12a and Cas9, as the Z-crRNA cannot be encoded genetically. As shown in FIG. 4d, zCRISPR-Cas12a demonstrated a significant improvement in multiplex gene editing as compared to CRISPR-Cas12a. It is noteworthy that as the number of guide RNAs increases, the Cas9 editing efficiency significantly decreases at site 15 and 20, while zCRISPR-Cas12a showed even better performance than CRISPR-Cas9 at five out of eight sites for octuple-gene editing in U2OS cell line (FIG. 5). The zCRISPR-Cas12a editing efficiency at site 9 exhibited a high level of effectiveness, with the efficiency up to of 94.3%, 90.6%, and 75.9% for quadruple, sextuple, and octuple-gene editing, respectively, in U2OS cell line. In contrast, Cas9 demonstrated lower efficiency, with rates of only 46.2%, 17.5%, and 6.8% for the same conditions. For the remaining sites tested in multiplex genome editing, zCRISPR-Cas12a performed comparable to or better than Cas9. Overall, the results indicate that zCRISPR-Cas12a has a greater capability for efficient multiplex genome editing in mammalian cells, as evidenced by the superior outcomes observed.

### DISCUSSION

In summary, our results provide an important proof-of-concept that the on-target potency of CRISPR nucleases can be augmented through enhancing the binding affinity between guide RNA and its DNA target. This enhanced editing capability is attributed to the stronger binding affinity offered by Z:T three-hydrogen-bonds pairing. By incorporating the noncanonical base Z into crRNA, the resultant zCRISPR-Cas12a showcased drastically improved on-target editing efficiency without compromising its low off-target effect virtue in mammalian cells, which is more advantageous than the most widely used CRISPR-Cas9 due to its comparable on-target editing efficiency, significantly lower off-target effect, and higher multiplexing capacity. However, the heightened binding affinity between Z-crRNA and target DNA could potentially induce extra off-target edits by Cas12a in the genome. To mitigate this risk, the high-fidelity Cas12a variants can be employed to significantly reduce the likelihood of off-target occurrences²⁶. As our data demonstrated the great compatibility of Z-crRNA for Cas12a variant, we believe Z-crRNA can further enhance the on-target efficiency of high-fidelity Cas12a variants while maintaining its upgraded specificity. Nevertheless, the Z-gRNA strategy was not able to enhance the editing efficiency of SpCas9, likely attributable to the intricate structure of the gRNA. Obtaining additional structural information could provide a deeper understanding of the underlying mechanism, aiding in the design of Z-crRNA for enhanced genome editing activity. We expect that our crRNA engineering strategy can be extended to other Cas proteins and CRISPR-based genome editing tools such as base editor, prime editor, and CRISPRa/i systems⁵²⁻⁵⁵.

### REFERENCES

1. Anzalone, A. V., Koblan, L. W. & Liu, D. R. Genome editing with CRISPR-Cas nucleases, base editors, transposases and prime editors. Nat Biotechnol 38, 824-844 (2020).
2. Dominguez, A. A., Lim, W. A. & Qi, L. S. Beyond editing: repurposing CRISPR-Cas9 for precision genome regulation and interrogation. Nat Rev Mol Cell Biol 17, 5-15 (2016).
3. Hsu, P. D., Lander, E. S. & Zhang, F. Development and applications of CRISPR-Cas9 for genome engineering. Cell 157, 1262-1278 (2014).
4. Wright, A. V., Nunez, J. K. & Doudna, J. A. Biology and Applications of CRISPR Systems: Harnessing Nature's Toolbox for Genome Engineering. Cell 164, 29-44 (2016).
5. Wang, J. Y. & Doudna, J. A. CRISPR technology: A decade of genome editing is only the beginning. Science 379, eadd8643 (2023).
6. Jinek, M. et al. A programmable dual-RNA-guided DNA endonuclease in adaptive bacterial immunity. Science 337, 816-821 (2012).
7. Mali, P. et al. RNA-guided human genome engineering via Cas9. Science 339, 823-826 (2013).
8. Kim, D. et al. Digenome-seq: genome-wide profiling of CRISPR-Cas9 off-target effects in human cells. Nat Methods 12, 237-243, 231 p following 243 (2015).
9. Nakade, S., Yamamoto, T. & Sakuma, T. Cas9, Cpf1 and C2c1/2/3-What's next? Bioengineered 8, 265-273 (2017).
10. Wang, H., La Russa, M. & Qi, L. S. CRISPR/Cas9 in Genome Editing and Beyond. Annu Rev Biochem 85, 227-264 (2016).
11. Kim, D. et al. Genome-wide analysis reveals specificities of Cpf1 endonucleases in human cells. Nat Biotechnol 34, 863-868 (2016).
12. Kleinstiver, B. P. et al. Genome-wide specificities of CRISPR-Cas Cpf1 nucleases in human cells. Nat Biotechnol 34, 869-874 (2016).
13. Strohkendl, I., Saifuddin, F. A., Rybarski, J. R., Finkelstein, I. J. & Russell, R. Kinetic Basis for DNA Target Specificity of CRISPR-Cas12a. Mol Cell 71, 816-824 e813 (2018).
14. Zhang, L. et al. Systematic in vitro profiling of off-target affinity, cleavage and efficiency for CRISPR enzymes. Nucleic Acids Res 48, 5037-5053 (2020).
15. Campa, C. C., Weisbach, N. R., Santinha, A. J., Incarnato, D. & Platt, R. J.

Multiplexed genome engineering by Cas12a and CRISPR arrays encoded on single transcripts. Nat Methods 16, 887-893 (2019).
16. Yan, W. X. et al. BLISS is a versatile and quantitative method for genome-wide profiling of DNA double-strand breaks. Nat Commun 8, 15058 (2017).
17. Allen, A. G. et al. A highly efficient transgene knock-in technology in clinically relevant cell types. Nat Biotechnol (2023).
18. Dai, X. et al. Massively parallel knock-in engineering of human T cells. Nat Biotechnol (2023).
19. Zetsche, B. et al. Cpf1 is a single RNA-guided endonuclease of a class 2 CRISPR-Cas system. Cell 163, 759-771 (2015).
20. Bin Moon, S. et al. Highly efficient genome editing by CRISPR-Cpf1 using CRISPR RNA with a uridinylate-rich 3′-overhang. Nat Commun 9, 3651 (2018).
21. Gier, R. A. et al. High-performance CRISPR-Cas12a genome editing for combinatorial genetic screening. Nat Commun 11, 3455 (2020).
22. Ling, X. et al. Improving the efficiency of CRISPR-Cas12a-based genome editing with site-specific covalent Cas12a-crRNA conjugates. Mol Cell 81, 4747-4756 e4747 (2021).
23. Safari, F., Zare, K., Negahdaripour, M., Barekati-Mowahed, M. & Ghasemi, Y. CRISPR Cpf1 proteins: structure, function and implications for genome editing. Cell Biosci 9, 36 (2019).
24. Zhang, L. et al. AsCas12a ultra nuclease facilitates the rapid generation of therapeutic cell medicines. Nat Commun 12, 3908 (2021).
25. Jones, S. K., Jr. et al. Massively parallel kinetic profiling of natural and engineered CRISPR nucleases. Nat Biotechnol 39, 84-93 (2021).
26. Kleinstiver, B. P. et al. Engineered CRISPR-Cas12a variants with increased activities and improved targeting ranges for gene, epigenetic and base editing. Nat Biotechnol 37, 276-282 (2019).
27. Liu, P. et al. Enhanced Cas12a editing in mammalian cells and zebrafish. Nucleic Acids Res 47, 4169-4180 (2019).
28. Gao, L. et al. Engineered Cpf1 variants with altered PAM specificities. Nat Biotechnol 35, 789-792 (2017).
29. Kocak, D. D. et al. Increasing the specificity of CRISPR systems with engineered RNA secondary structures. Nat Biotechnol 37, 657-666 (2019).
30. Li, B. et al. Engineering CRISPR-Cpf1 crRNAs and mRNAs to maximize genome editing efficiency. Nat Biomed Eng 1 (2017).
31. Cromwell, C. R. et al. Incorporation of bridged nucleic acids into CRISPR RNAs improves Cas9 endonuclease specificity. Nat Commun 9, 1448 (2018).
32. Rueda, F. O. et al. Mapping the sugar dependency for rational generation of a DNA-RNA hybrid-guided Cas9 endonuclease. Nat Commun 8, 1610 (2017).
33. Hendel, A. et al. Chemically modified guide RNAs enhance CRISPR-Cas genome editing in human primary cells. Nat Biotechnol 33, 985-989 (2015).
34. Ryan, D. E. et al. Improving CRISPR-Cas specificity with chemical modifications in single-guide RNAs. Nucleic Acids Res 46, 792-803 (2018).
35. Krysler, A. R., Cromwell, C. R., Tu, T., Jovel, J. & Hubbard, B. P. Guide RNAs containing universal bases enable Cas9/Cas12a recognition of polymorphic sequences. Nat Commun 13, 1617 (2022).
36. Yang, H. et al. CRISPR-Cas9 recognition of enzymatically synthesized base-modified nucleic acids. Nucleic Acids Res 51, 1501-1511 (2023).
37. Gao, S. et al. Harnessing non-Watson-Crick's base pairing to enhance CRISPR effectors cleavage activities and enable gene editing in mammalian cells. Proc Natl Acad Sci USA 121, e2308415120 (2024).
38. Grome, M. W. & Isaacs, F. J. ZTCG: Viruses expand the genetic alphabet. Science 372, 460-461 (2021).
39. Pezo, V. et al. Noncanonical DNA polymerization by aminoadenine-based siphoviruses. Science 372, 520-524 (2021).
40. Sleiman, D. et al. A third purine biosynthetic pathway encoded by aminoadenine-based viral DNA genomes. Science 372, 516-520 (2021).
41. Zhou, Y. et al. A widespread pathway for substitution of adenine by diaminopurine in phage genomes. Science 372, 512-516 (2021).
42. Wang, J., Pan, X. & Liang, X. Assessment for Melting Temperature Measurement of Nucleic Acid by HRM. J Anal Methods Chem 2016, U.S. Pat. No. 5,318,935 (2016).
43. Rozners, E. Chemical Modifications of CRISPR RNAs to Improve Gene-Editing Activity and Specificity. J Am Chem Soc 144, 12584-12594 (2022).
44. Tsai, S. Q. et al. GUIDE-seq enables genome-wide profiling of off-target cleavage by CRISPR-Cas nucleases. Nat Biotechnol 33, 187-197 (2015).
45. Chen, P. et al. A Cas12a ortholog with stringent PAM recognition followed by low off-target editing rates for genome editing. Genome Biol 21, 78 (2020).
46. Zetsche, B., Abudayyeh, O. O., Gootenberg, J. S., Scott, D. A. & Zhang, F. A Survey of Genome Editing Activity for 16 Cas12a Orthologs. Keio J Med 69, 59-65 (2020).
47. Teng, F. et al. Enhanced mammalian genome editing by new Cas12a orthologs with optimized crRNA scaffolds. Genome Biol 20, 15 (2019).
48. Long T. Nguyen, N. C. M., Piyush K. Jain A Combinatorial Approach towards Adaptability of 22 Functional Cas12a Orthologs for Nucleic Acid Detection in Clinical Samples. bioRxiv (2021).
49. Yu, Y. et al. An efficient gene knock-in strategy using 5′-modified double-stranded DNA donors with short homology arms. Nat Chem Biol 16, 387-390 (2020).
50. Medert, R. et al. Efficient single copy integration via homology-directed repair (scHDR) by 5′modification of large DNA donor fragments in mice. Nucleic Acids Res (2022).
51. McCarty, N. S., Graham, A. E., Studena, L. & Ledesma-Amaro, R. Multiplexed CRISPR technologies for gene editing and transcriptional regulation. Nat Commun 11, 1281 (2020).
52. Li, X. et al. Base editing with a Cpf1-cytidine deaminase fusion. Nat Biotechnol 36, 324-327 (2018).
53. Jensen, T. I. et al. Targeted regulation of transcription in primary cells using CRISPRa and CRISPRi. Genome Res 31, 2120-2130 (2021).
54. Anzalone, A. V. et al. Search-and-replace genome editing without double-strand breaks or donor DNA. Nature 576, 149-157 (2019).
55. Liang, R. et al. Prime editing using CRISPR-Cas12a and circular RNAs in human cells. Nat Biotechnol (2024).
56. Malinin, N. L. et al. Defining genome-wide CRISPR-Cas genome-editing nuclease activity with GUIDE-seq. Nat Protoc 16, 5592-5615 (2021).
57. Xin, C. et al. Comprehensive assessment of miniature CRISPR-Cas12f nucleases for gene disruption. Nat Commun 13, 5623 (2022).

### Example 3 Characterization of the zCRISPR-Cas12a System In Vitro

To synthesize the Z containing crRNA (Z-crRNA), we replaced adenosine-5′-triphosphate (ATP) with 2-adenosine-5′-triphosphate (ZTP) in the in vitro transcription (IVT) reaction and used SP6 RNA polymerase to incorporate Z into crRNA (FIG. 6a). Agarose gel electrophoresis showed the resultant Z-crRNA had a correct size compared to the canonical A-based crRNA (A-crRNA). However, its band intensity was much lower than that of A-crRNA despite with the same amount of RNA post purification (FIG. 6b). This is likely due to the low dye intercalation rate of Z containing crRNA.

Given that PAM sequence may affect the Cas12a cleavage efficiency, we sought to investigate the performance of zCRISPR-Cas12a on all types of PAMs. A site (site 7 in FIG. 9B) with significant improvement in Cas12a cleavage efficiency by using Z-crRNA was selected for validation. The original TTTT PAM of the target site was mutagenized to TTTA, TTTG, and TTTC PAMs on the plasmid, respectively. In vitro cleavage assays were applied to test the cleavage efficiency. We observed enhanced cleavage efficiency using zCRISPR-Cas12a on all four types of PAMs (FIG. 9C). We also noticed that the Cas12a cleavage mediated by A-crRNA was more efficient on TTTA, TTTG, and TTTC PAMs, which is consistent with previous report¹.

To further investigate the applicability of zCRISPR-Cas12a system, we tested its efficacy on the endogenous gene fragment. DNA fragments were amplified from the DNMT1 gene in the human genome and ten sites with TTTT PAM were investigated by in vitro cleavage assay. The results indicate that the cleavage efficiency was improved by zCRISPR-Cas12a at five sites, and the rest of the sites had a similar cleavage efficiency as CRISPR-Cas12a (FIG. 9D). Additionally, we investigated whether zCRISPR-Cas12a is generally applicable for different Cas12a isoforms, AsCas12a and LbCas12a. We applied the same in vitro cleavage assay on ten sites with two types of Cas12a. The result indicates that zCRISPR-Cas12a works for different Cas12a proteins (FIG. 9E).

### DISCUSSION

Although Cas12a has the potential to be a superior alternative to Cas9 due to its intrinsically higher precision and multiplexability in genome editing, its performance for on-target editing is unsatisfactory, which has hampered its broad application. To overcome this obstacle, several strategies based on protein engineering¹⁻⁴ and crRNA engineering⁵⁻⁸ were developed to enhance its editing efficiency, but with limited success. For example, some previously reported Cas12a variants, enAsCas12a³ and iCas12a⁹, only showed 2-5-fold improvements in genome editing efficiency, and the enhancement of editing efficiency on some low-efficiency-sites was low. Similarly, crRNA engineering strategies also achieved limited improvement of on-target editing efficiency^{5,8,10}. Moreover, most of these protein and crRNA engineering approaches are time-consuming and labor-intensive. A simple and generally applicable strategy is highly desirable.

Chemical modifications of the base in guide RNA are rare, and most modifications of bases in guide RNA showed limited enhancement or had an adverse impact on cleavage efficiency^{12,13}. It is important to note that base Z stands out from other artificially chemical modified bases as it is a naturally occurring base.

We systematically characterized the performance of zCRISPR-Cas12a in mammalian cell genome editing and deciphered the general rules for Z-crRNA design to achieve higher on-target editing efficiency. We applied our strategy to several previously reported low-efficiency-sites for validation, the results demonstrated that the on-target editing efficiency of those sites was improved up to a hundred-fold compared to A-crRNA mediated Cas12a. Our side-by-side comparison experiment showed that zCRISPR-Cas12a achieved an on-target editing efficiency comparable to that of the CRISPR-Cas9 system but with much lower off-target in mammalian cells. Moreover, three cell lines were harnessed to validate the efficacy of zCRISPR-Cas12a in this study, all of them exhibited the consistent result that zCRISPR-Cas12a can dramatically enhance the on-target editing efficiency. Additionally, the upgraded Cas12a system can be utilized not only for editing a single site but also for achieving accurate gene knock-in and editing multiple genes simultaneously. However, there are limitations of using Z-crRNA, as base Z cannot be genetically encoded and can only be incorporated into crRNA in vitro. To expand the delivery options, potential approaches include co-delivering CRISPR-Cas mRNA and Z-crRNA ex vivo or in vivo using lipid nanoparticle (LNP)-based RNA delivery methods¹⁴ or LNP-mediated RNP delivery¹⁵. Given Cas12a has ability to process its own crRNA¹⁶, co-delivery of Cas12a mRNA and crRNA arrays into mammalian cells for multiplexed gene editing is feasible.

### REFERENCES

1 Zhang, L. et al. AsCas12a ultra nuclease facilitates the rapid generation of therapeutic cell medicines. Nat Commun 12, 3908, doi:10.1038/s41467-021-24017-8 (2021).
2 Jones, S. K., Jr. et al. Massively parallel kinetic profiling of natural and engineered CRISPR nucleases. Nat Biotechnol 39, 84-93, doi:10.1038/s41587-020-0646-5 (2021).
3 Kleinstiver, B. P. et al. Engineered CRISPR-Cas12a variants with increased activities and improved targeting ranges for gene, epigenetic and base editing. Nat Biotechnol 37, 276-282, doi:10.1038/s41587-018-0011-0 (2019).
4 Liu, P. et al. Enhanced Cas12a editing in mammalian cells and zebrafish. Nucleic Acids Res 47, 4169-4180, doi:10.1093/nar/gkz184 (2019).
Bin Moon, S. et al. Highly efficient genome editing by CRISPR-Cpf1 using CRISPR RNA with a uridinylate-rich 3′-overhang. Nat Commun 9, 3651, doi:10.1038/s41467-018-06129-w (2018).
6 Kocak, D. D. et al. Increasing the specificity of CRISPR systems with engineered RNA secondary structures. Nat Biotechnol 37, 657-666, doi:10.1038/s41587-019-0095-1 (2019).
7 Li, B. et al. Engineering CRISPR-Cpf1 crRNAs and mRNAs to maximize genome editing efficiency. Nat Biomed Eng 1, doi:10.1038/s41551-017-0066 (2017).
8 Ling, X. et al. Improving the efficiency of CRISPR-Cas12a-based genome editing with site-specific covalent Cas12a-crRNA conjugates. Mol Cell 81, 4747-4756 e4747, doi:10.1016/j.molcel.2021.09.021 (2021).
9 Ma, E. et al. Improved genome editing by an engineered CRISPR-Cas12a. Nucleic Acids Res 50, 12689-12701, doi:10.1093/nar/gkac1192 (2022).
Kim, H. et al. Highly specific chimeric DNA-RNA-guided genome editing with enhanced CRISPR-Cas12a system. Mol Ther Nucleic Acids 28, 353-362, doi:10.1016/j.omtn.2022.03.021 (2022).
11 Allen, D., Rosenberg, M. & Hendel, A. Using Synthetically Engineered Guide RNAs to Enhance CRISPR Genome Editing Systems in Mammalian Cells. Front Genome Ed 2, 617910, doi:10.3389/fgeed.2020.617910 (2020).
12 Krysler, A. R., Cromwell, C. R., Tu, T., Jovel, J. & Hubbard, B. P. Guide RNAs containing universal bases enable Cas9/Cas12a recognition of polymorphic sequences. Nat Commun 13, 1617, doi:10.1038/s41467-022-29202-x (2022).
13 Yang, H. et al. CRISPR-Cas9 recognition of enzymatically synthesized base-modified nucleic acids. Nucleic Acids Res 51, 1501-1511, doi:10.1093/nar/gkac1147 (2023).
14 Gillmore, J. D. et al. CRISPR-Cas9 In Vivo Gene Editing for Transthyretin Amyloidosis. N Engl J Med 385, 493-502, doi:10.1056/NEJMoa2107454 (2021).
Mirjalili Mohanna, S. Z. et al. LNP-mediated delivery of CRISPR RNP for wide-spread in vivo genome editing in mouse cornea. J Control Release 350, 401-413, doi:10.1016/j.jconrel.2022.08.042 (2022).
16 Port, F., Starostecka, M. & Boutros, M. Multiplexed conditional genome editing with Cas12a in Drosophila. Proc Natl Acad Sci USA 117, 22890-22899, doi:10.1073/pnas.2004655117 (2020).
17 Kleinstiver, B. P. et al. Genome-wide specificities of CRISPR-Cas Cpf1 nucleases in human cells. Nat Biotechnol 34, 869-874, doi:10.1038/nbt.3620 (2016).

1. A recombinant clustered regularly interspaced short palindromic repeat (CRISPR) RNA (crRNA) comprising a scaffold region and a spacer region, wherein one or more 2-aminoadenine nucleotides are present in the spacer region of the crRNA.

2. The recombinant crRNA of claim 1, wherein the one or more 2-aminoadenine nucleotides are substituted for one or more adenosine nucleotides.

3. The recombinant crRNA of claim 1, wherein the crRNA comprises 2, 3, 4, 5, 6, 7, or more 2-aminoadenine nucleotides in the spacer region of the crRNA.

4. The recombinant crRNA of claim 1, wherein the crRNA comprises 7 or more 2-aminoadenine nucleotides in the spacer region of the crRNA.

5. A recombinant clustered regularly interspaced short palindromic repeat (CRISPR)-associated (Cas) system comprising (i) one or more crRNAs of claim 1, wherein the one or more crRNAs are capable of specifically hybridizing to one or more target nucleic acid molecules, and (ii) a Cas12a protein.

6. (canceled)

7. The recombinant CRISPR-Cas system of claim 5 further comprising one or more single-stranded donor nucleic acid molecules and/or double-stranded donor nucleic acid molecules.

8. A method of modifying a cell comprising delivering to the cell the recombinant clustered regularly interspaced short palindromic repeat (CRISPR)-associated (Cas) system of claim 5 wherein the spacer regions comprise one or more 2-aminoadenine nucleotides.

9. (canceled)

10. (canceled)

11. (canceled)

12. The method of claim 8, wherein the method further comprises delivering to the cell a donor nucleic acid molecule.

13. (canceled)

14. (canceled)

15. The method of claim 8, wherein the one or more crRNAs comprise 2, 3, 4, 5, 6, 7, or more 2-aminoadenine nucleotides in the spacer region.

16.-20. (canceled)

21. The method of claim 8, wherein the one or more crRNAs comprise two or more spacer regions, and wherein the Cas12a protein can create a deletion of the one or more target nucleic acid molecules.

22. The method of claim 8, wherein the Cas12a protein and the one or more crRNAs are allowed to associate prior to delivery to the cell.

23. A method for site-specific integration of a donor nucleic acid molecule into a target nucleic acid molecule, comprising introducing into a cell: (i) a Cas12a protein; (ii) one or more crRNA molecules comprising a scaffold region and one or more spacer regions capable of hybridizing to the target nucleic acid molecule, wherein the one or more spacer regions comprise one or more 2-aminoadenine nucleotides, and (iii) a donor nucleic acid molecule; wherein the donor nucleic acid molecule is inserted into the target nucleic acid molecule at a site-specific target site; or
a method of reducing expression of a target gene in a cell comprising introducing into the cell (a) one or more crRNA molecules comprising a scaffold region and one or more spacer regions capable of hybridizing to the target gene, wherein the one or more spacer regions comprise one or more 2-aminoadenine nucleotides and (b) a Cas12a protein, wherein the one or more crRNA molecules hybridize to the target gene and the Cas12a cleaves the target gene, such that the target gene expression is reduced in the cell relative to a cell in which the one or more crRNAs and the Cas12a protein is not introduced.

24.-35. (canceled)

36. The method of claim 23, wherein the Cas12a protein and the one or more crRNAs are allowed to associate prior to delivery to the cell.

37.-46. (canceled)

47. The method of claim 37, wherein the crRNA comprises two or more spacer regions, and wherein the Cas12a protein can create a deletion of the one or more target nucleic acid molecules.

48. The method of claim 37, wherein the Cas12a protein and the one or more crRNAs are allowed to associate prior to delivery to the cell.

49. A pharmaceutical composition comprising a complex comprising (i) one or more crRNA molecules comprising a scaffold region and one or more spacer regions capable of hybridizing to a target gene, wherein the one or more spacer regions comprise one or more 2-aminoadenine nucleotides; (ii) a Cas12a protein; and a pharmaceutical carrier or excipient.

50. The recombinant clustered regularly interspaced short palindromic repeat (CRISPR) RNA (crRNA) of claim 1, wherein the crRNA is present in a lipid nanoparticle or lipoplex.

51. The recombinant clustered regularly interspaced short palindromic repeat (CRISPR)-associated (Cas) system of claim 5, wherein the one or more crRNAs, the Cas12a protein, or both are present in a lipid nanoparticle or lipoplex.

52. The method of claim 23, wherein the Cas12a protein, the one or more crRNA molecules, the donor nucleic acid molecule, or combinations thereof are present in a lipid nanoparticle or lipoplex.

53. (canceled)

54. The pharmaceutical composition of claim 49, wherein the one or more crRNA molecules, the Cas12a protein, or both are present in a lipid nanoparticle or lipoplex.


SEQUENCE LISTING (522 sequences)

SEQ ID NO: 1 - DNA, 21 bp, synthetic construct
  source 1..21: mol_type = other DNA
ttggagttcgttttcttcctt

SEQ ID NO: 2 - DNA, 21 bp, synthetic construct
  source 1..21: mol_type = other DNA
ttagttcatttttccctttgt

SEQ ID NO: 3 - DNA, 21 bp, synthetic construct
  source 1..21: mol_type = other DNA
tcatctattttaccttctgtg

SEQ ID NO: 4 - DNA, 21 bp, synthetic construct
  source 1..21: mol_type = other DNA
tgtatacgatcagttgtgggg

SEQ ID NO: 5 - DNA, 21 bp, synthetic construct
  source 1..21: mol_type = other DNA
tacaggcatcactttagtttc

SEQ ID NO: 6 - DNA, 21 bp, synthetic construct
  source 1..21: mol_type = other DNA
cagatccaggcaagtatcttg

SEQ ID NO: 7 - DNA, 21 bp, synthetic construct
  source 1..21: mol_type = other DNA
acaattataagatctctgttt

SEQ ID NO: 8 - DNA, 21 bp, synthetic construct
  source 1..21: mol_type = other DNA
aagacaggacacgtattcacc

SEQ ID NO: 9 - DNA, 21 bp, synthetic construct
  source 1..21: mol_type = other DNA
ttctaaatgataataaatact

SEQ ID NO: 10 - DNA, 21 bp, synthetic construct
  source 1..21: mol_type = other DNA
taaatacattcaaatatgtat

SEQ ID NO: 11 - DNA, 21 bp, synthetic construct
  source 1..21: mol_type = other DNA
atatcaaaaactgattttccc

SEQ ID NO: 12 - DNA, 21 bp, synthetic construct
  source 1..21: mol_type = other DNA
ttaaagagaattaagaaaata

SEQ ID NO: 13 - DNA, 21 bp, synthetic construct
  source 1..21: mol_type = other DNA
acaacacagaaagagtttgta

SEQ ID NO: 14 - DNA, 21 bp, synthetic construct
  source 1..21: mol_type = other DNA
tcttctctctgggtctgggcc

SEQ ID NO: 15 - DNA, 21 bp, synthetic construct
  source 1..21: mol_type = other DNA
ttggagttcgttttcttcctt

SEQ ID NO: 16 - DNA, 21 bp, synthetic construct
  source 1..21: mol_type = other DNA
ttagttcatttttccctttgt

SEQ ID NO: 17 - DNA, 21 bp, synthetic construct
  source 1..21: mol_type = other DNA
tcatctattttaccttctgtg

SEQ ID NO: 18 - DNA, 21 bp, synthetic construct
  source 1..21: mol_type = other DNA
tgtatacgatcagttgtgggg

SEQ ID NO: 19 - DNA, 21 bp, synthetic construct
  source 1..21: mol_type = other DNA
tacaggcatcactttagtttc

SEQ ID NO: 20 - DNA, 21 bp, synthetic construct
  source 1..21: mol_type = other DNA
cagatccaggcaagtatcttg

SEQ ID NO: 21 - DNA, 21 bp, synthetic construct
  source 1..21: mol_type = other DNA
acaattataagatctctgttt

SEQ ID NO: 22 - DNA, 21 bp, synthetic construct
  source 1..21: mol_type = other DNA
aagacaggacacgtattcacc

SEQ ID NO: 23 - DNA, 21 bp, synthetic construct
  source 1..21: mol_type = other DNA
gaaataaggtgtcctaatgaa

SEQ ID NO: 24 - DNA, 21 bp, synthetic construct
  source 1..21: mol_type = other DNA
agcaaagcaaatgtacaggac

SEQ ID NO: 25 - DNA, 21 bp, synthetic construct
  source 1..21: mol_type = other DNA
taaggcaaggagacataaaga

SEQ ID NO: 26 - DNA, 21 bp, synthetic construct
  source 1..21: mol_type = other DNA
gaagaataactatatagaaca

SEQ ID NO: 27 - DNA, 16 bp, synthetic construct
  source 1..16: mol_type = other DNA
ccaaattaagctgcct

SEQ ID NO: 28 - DNA, 17 bp, synthetic construct
  source 1..17: mol_type = other DNA
ccaaattaagctgccta

SEQ ID NO: 29 - DNA, 18 bp, synthetic construct
  source 1..18: mol_type = other DNA
ccaaattaagctgcctaa

SEQ ID NO: 30 - DNA, 19 bp, synthetic construct
  source 1..19: mol_type = other DNA
ccaaattaagctgcctaat

SEQ ID NO: 31 - DNA, 20 bp, synthetic construct
  source 1..20: mol_type = other DNA
ccaaattaagctgcctaatg

SEQ ID NO: 32 - DNA, 21 bp, synthetic construct
  source 1..21: mol_type = other DNA
ccaaattaagctgcctaatgt

SEQ ID NO: 33 - DNA, 22 bp, synthetic construct
  source 1..22: mol_type = other DNA
ccaaattaagctgcctaatgtt

SEQ ID NO: 34 - DNA, 16 bp, synthetic construct
  source 1..16: mol_type = other DNA
cagaatatataaagaa

SEQ ID NO: 35 - DNA, 17 bp, synthetic construct
  source 1..17: mol_type = other DNA
cagaatatataaagaaa

SEQ ID NO: 36 - DNA, 18 bp, synthetic construct
  source 1..18: mol_type = other DNA
cagaatatataaagaaac

SEQ ID NO: 37 - DNA, 19 bp, synthetic construct
  source 1..19: mol_type = other DNA
cagaatatataaagaaaca

SEQ ID NO: 38 - DNA, 20 bp, synthetic construct
  source 1..20: mol_type = other DNA
cagaatatataaagaaacat

SEQ ID NO: 39 - DNA, 21 bp, synthetic construct
  source 1..21: mol_type = other DNA
cagaatatataaagaaacatt

SEQ ID NO: 40 - DNA, 22 bp, synthetic construct
  source 1..22: mol_type = other DNA
cagaatatataaagaaacatta

SEQ ID NO: 41 - DNA, 20 bp, synthetic construct
  source 1..20: mol_type = other DNA
atctataaaaagggggtggg

SEQ ID NO: 42 - DNA, 20 bp, synthetic construct
  source 1..20: mol_type = other DNA
taagaatcagcgtgtggttg

SEQ ID NO: 43 - DNA, 20 bp, synthetic construct
  source 1..20: mol_type = other DNA
aaagacagttttgctctggg

SEQ ID NO: 44 - DNA, 20 bp, synthetic construct
  source 1..20: mol_type = other DNA
ttaaaaagactgttttggct

SEQ ID NO: 45 - DNA, 20 bp, synthetic construct
  source 1..20: mol_type = other DNA
taagaataatactgtgcttc

SEQ ID NO: 46 - DNA, 20 bp, synthetic construct
  source 1..20: mol_type = other DNA
cagtaagcaaactggcttcc

SEQ ID NO: 47 - DNA, 20 bp, synthetic construct
  source 1..20: mol_type = other DNA
ccataaataaatgctgcctg

SEQ ID NO: 48 - DNA, 20 bp, synthetic construct
  source 1..20: mol_type = other DNA
ctgctgttcctgggcaaaac

SEQ ID NO: 49 - DNA, 20 bp, synthetic construct
  source 1..20: mol_type = other DNA
ttttgttttttaataagatg

SEQ ID NO: 50 - DNA, 20 bp, synthetic construct
  source 1..20: mol_type = other DNA
tgctttgttgtgaaactgaa

SEQ ID NO: 51 - DNA, 20 bp, synthetic construct
  source 1..20: mol_type = other DNA
tctgctttttgtattaaacc

SEQ ID NO: 52 - DNA, 20 bp, synthetic construct
  source 1..20: mol_type = other DNA
tccgttctgggggaaaaaaa

SEQ ID NO: 53 - DNA, 20 bp, synthetic construct
  source 1..20: mol_type = other DNA
ctgttcctctcaaacaaagg

SEQ ID NO: 54 - DNA, 20 bp, synthetic construct
  source 1..20: mol_type = other DNA
tcttccttgcacccaaacag

SEQ ID NO: 55 - DNA, 21 bp, synthetic construct
  source 1..21: mol_type = other DNA
aaaaaggatttccatctatta

SEQ ID NO: 56 - DNA, 21 bp, synthetic construct
  source 1..21: mol_type = other DNA
aaaactacaacaaacttactt

SEQ ID NO: 57 - DNA, 21 bp, synthetic construct
  source 1..21: mol_type = other DNA
aaagtttccaaacgaccccag

SEQ ID NO: 58 - DNA, 21 bp, synthetic construct
  source 1..21: mol_type = other DNA
gaaaacctaaagaagagataa

SEQ ID NO: 59 - DNA, 21 bp, synthetic construct
  source 1..21: mol_type = other DNA
aatcacattatgggattgcta

SEQ ID NO: 60 - DNA, 21 bp, synthetic construct
  source 1..21: mol_type = other DNA
tacaacggctcaaagaactgc

SEQ ID NO: 61 - DNA, 21 bp, synthetic construct
  source 1..21: mol_type = other DNA
aataggtaagtacatcttttg

SEQ ID NO: 62 - DNA, 21 bp, synthetic construct
  source 1..21: mol_type = other DNA
atcactcctggtatatagaaa

SEQ ID NO: 63 - DNA, 21 bp, synthetic construct
  source 1..21: mol_type = other DNA
cagaatatataaagaaacatt

SEQ ID NO: 64 - DNA, 20 bp, synthetic construct
  source 1..20: mol_type = other DNA
ctgatggtccatgtctgtta

SEQ ID NO: 65 - DNA, 20 bp, synthetic construct
  source 1..20: mol_type = other DNA
gctcagcaggcacctgcctc

SEQ ID NO: 66 - DNA, 20 bp, synthetic construct
  source 1..20: mol_type = other DNA
ggcggggtccagttccggga

SEQ ID NO: 67 - DNA, 20 bp, synthetic construct
  source 1..20: mol_type = other DNA
gtcggcatggccccattcgc

SEQ ID NO: 68 - DNA, 20 bp, synthetic construct
  source 1..20: mol_type = other DNA
ggatgccactaaaagggaaa

SEQ ID NO: 69 - DNA, 20 bp, synthetic construct
  source 1..20: mol_type = other DNA
gggtgatcagacccaacagc

SEQ ID NO: 70 - DNA, 20 bp, synthetic construct
  source 1..20: mol_type = other DNA
ggcaaataggaatggcaaga

SEQ ID NO: 71 - DNA, 20 bp, synthetic construct
  source 1..20: mol_type = other DNA
gagctgcttaagcatttcaa

SEQ ID NO: 72 - DNA, 20 bp, synthetic construct
  source 1..20: mol_type = other DNA
ggcatgaattataatgctgt

SEQ ID NO: 73 - DNA, 20 bp, synthetic construct
  source 1..20: mol_type = other DNA
gaatctcaaaaataaaagac

SEQ ID NO: 74 - DNA, 20 bp, synthetic construct
  source 1..20: mol_type = other DNA
gagaaaatatgggttgaggt

SEQ ID NO: 75 - DNA, 18 bp, synthetic construct
  source 1..18: mol_type = other DNA
ttgatggtacatgacaag

SEQ ID NO: 76 - DNA, 18 bp, synthetic construct
  source 1..18: mol_type = other DNA
atagtcattccaaatatg

SEQ ID NO: 77 - DNA, 18 bp, synthetic construct
  source 1..18: mol_type = other DNA
aaagggaactgctgacaa

SEQ ID NO: 78 - DNA, 18 bp, synthetic construct
  source 1..18: mol_type = other DNA
taataagcaatcaaggtt

SEQ ID NO: 79 - DNA, 18 bp, synthetic construct
  source 1..18: mol_type = other DNA
aacaaatttcattgcact

SEQ ID NO: 80 - DNA, 18 bp, synthetic construct
  source 1..18: mol_type = other DNA
gaaatataattgacagga

SEQ ID NO: 81 - DNA, 20 bp, synthetic construct
  source 1..20: mol_type = other DNA
gattcattctcagtgccatg

SEQ ID NO: 82 - DNA, 20 bp, synthetic construct
  source 1..20: mol_type = other DNA
agaacacatacccctgggcc

SEQ ID NO: 83 - DNA, 20 bp, synthetic construct
  source 1..20: mol_type = other DNA
cacataggccattcagaaac

SEQ ID NO: 84 - DNA, 21 bp, synthetic construct
  source 1..21: mol_type = other DNA
caatattattgaagcatttat

SEQ ID NO: 85 - DNA, 21 bp, synthetic construct
  source 1..21: mol_type = other DNA
cgattgatgaacacctataat

SEQ ID NO: 86 - DNA, 21 bp, synthetic construct
  source 1..21: mol_type = other DNA
ctcatttataaggttaaataa

SEQ ID NO: 87 - DNA, 21 bp, synthetic construct
  source 1..21: mol_type = other DNA
gtatatacaatatttctagtt

SEQ ID NO: 88 - DNA, 21 bp, synthetic construct
  source 1..21: mol_type = other DNA
gatatcaaaattatacatgtc

SEQ ID NO: 89 - DNA, 21 bp, synthetic construct
  source 1..21: mol_type = other DNA
attaggaaaggacagtgggag

SEQ ID NO: 90 - DNA, 21 bp, synthetic construct
  source 1..21: mol_type = other DNA
aaccaataggccgaaatcggc

SEQ ID NO: 91 - DNA, 21 bp, synthetic construct
  source 1..21: mol_type = other DNA
gcaaaaagctcccgggagctt

SEQ ID NO: 92 - DNA, 21 bp, synthetic construct
  source 1..21: mol_type = other DNA
ctaaatacattcaaatatgta

SEQ ID NO: 93 - DNA, 21 bp, synthetic construct
  source 1..21: mol_type = other DNA
ggcgccaaacagcccgggcac

SEQ ID NO: 94 - DNA, 21 bp, synthetic construct
  source 1..21: mol_type = other DNA
gatccccaaatacagcaagct

SEQ ID NO: 95 - DNA, 21 bp, synthetic construct
  source 1..21: mol_type = other DNA
ggcaggaactttaaccgtgca

SEQ ID NO: 96 - DNA, 21 bp, synthetic construct
  source 1..21: mol_type = other DNA
cctgtaaatctgtgcctgtga

SEQ ID NO: 97 - DNA, 21 bp, synthetic construct
  source 1..21: mol_type = other DNA
caggaatgaactgatggcgtt

SEQ ID NO: 98 - DNA, 21 bp, synthetic construct
  source 1..21: mol_type = other DNA
gagacagggtctcactctgtc

SEQ ID NO: 99 - DNA, 21 bp, synthetic construct
  source 1..21: mol_type = other DNA
gggagaatactggcacagatg

SEQ ID NO: 100 - DNA, 21 bp, synthetic construct
  source 1..21: mol_type = other DNA
aaatcaagttttattttggga

SEQ ID NO: 101 - DNA, 21 bp, synthetic construct
  source 1..21: mol_type = other DNA
aagagacaggatctcactgtg

SEQ ID NO: 102 - DNA, 21 bp, synthetic construct
  source 1..21: mol_type = other DNA
aaggattaacttgggcatggt

SEQ ID NO: 103 - DNA, 21 bp, synthetic construct
  source 1..21: mol_type = other DNA
gagatttattttcttaattct

SEQ ID NO: 104 - DNA, 21 bp, synthetic construct
  source 1..21: mol_type = other DNA
ttaaatcagctcattttttaa

SEQ ID NO: 105 - DNA, 21 bp, synthetic construct
  source 1..21: mol_type = other DNA
tttttctaaatacattcaaat

SEQ ID NO: 106 - DNA, 21 bp, synthetic construct
  source 1..21: mol_type = other DNA
ttttcttaattctctttaaca

SEQ ID NO: 107 - DNA, 21 bp, synthetic construct
  source 1..21: mol_type = other DNA
acaaataaagcatttttttca

SEQ ID NO: 108 - DNA, 21 bp, synthetic construct
  source 1..21: mol_type = other DNA
tgattttttgtatatacaata

SEQ ID NO: 109 - DNA, 21 bp, synthetic construct
  source 1..21: mol_type = other DNA
ttattttcgagatttattttc

SEQ ID NO: 110 - DNA, 21 bp, synthetic construct
  source 1..21: mol_type = other DNA
atttataagggattttgccga

SEQ ID NO: 111 - DNA, 21 bp, synthetic construct
  source 1..21: mol_type = other DNA
tgaaatttgtgatgctattgc

SEQ ID NO: 112 - DNA, 21 bp, synthetic construct
  source 1..21: mol_type = other DNA
ctcattttattaggaaaggac

SEQ ID NO: 113 - DNA, 21 bp, synthetic construct
  source 1..21: mol_type = other DNA
ataataatgaaactggcgagt

SEQ ID NO: 114 - DNA, 21 bp, synthetic construct
  source 1..21: mol_type = other DNA
gctgatacttaaacagagacc

SEQ ID NO: 115 - DNA, 21 bp, synthetic construct
  source 1..21: mol_type = other DNA
attattattaaagaatccatt

SEQ ID NO: 116 - DNA, 21 bp, synthetic construct
  source 1..21: mol_type = other DNA
caagaatcagggacactgtag

SEQ ID NO: 117 - DNA, 21 bp, synthetic construct
  source 1..21: mol_type = other DNA
taattttagtagagacggagt

SEQ ID NO: 118 - DNA, 21 bp, synthetic construct
  source 1..21: mol_type = other DNA
aatgtcagctcaacacagcct

SEQ ID NO: 119 - DNA, 21 bp, synthetic construct
  source 1..21: mol_type = other DNA
agtagagacggagtttcacca

SEQ ID NO: 120 - DNA, 21 bp, synthetic construct
  source 1..21: mol_type = other DNA
ccaagaatcagggacactgta

SEQ ID NO: 121 - DNA, 21 bp, synthetic construct
  source 1..21: mol_type = other DNA
ccaaattaagctgcctaatgt

SEQ ID NO: 122 - DNA, 21 bp, synthetic construct
  source 1..21: mol_type = other DNA
ggattagtacggatcagccag

SEQ ID NO: 123 - DNA, 21 bp, synthetic construct
  source 1..21: mol_type = other DNA
cattttacagttttaccaaat

SEQ ID NO: 124 - DNA, 21 bp, synthetic construct
  source 1..21: mol_type = other DNA
cagaatatataaagaaacatt

SEQ ID NO: 125 - DNA, 21 bp, synthetic construct
  source 1..21: mol_type = other DNA
cagcatcaataacattgatgt

SEQ ID NO: 126 - DNA, 21 bp, synthetic construct
  source 1..21: mol_type = other DNA
aaacagtgagttaaaatctgg

SEQ ID NO: 127 - DNA, 21 bp, synthetic construct
  source 1..21: mol_type = other DNA
agaggctgaggcaggaagact

SEQ ID NO: 128 - DNA, 21 bp, synthetic construct
  source 1..21: mol_type = other DNA
aaattgtttgcagcatcaata

SEQ ID NO: 129 - DNA, 20 bp, synthetic construct
  source 1..20: mol_type = other DNA
ttgatggtacatgacaaggt

SEQ ID NO: 130 - DNA, 20 bp, synthetic construct
  source 1..20: mol_type = other DNA
aacaaatttcattgcacttt

SEQ ID NO: 131 - DNA, 20 bp, synthetic construct
  source 1..20: mol_type = other DNA
gaaatataattgacaggatt

SEQ ID NO: 132 - DNA, 39 bp, synthetic construct
  source 1..39: mol_type = other DNA
aatttaggtgacactatagtaatttctactcttgtagat

SEQ ID NO: 133 - DNA, 6464 bp, synthetic construct
  source 1..6464: mol_type = other DNA
ctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagttattaatagtaatcaattacggggtcattagttcatagcccatatatggagttccgcgttacatacccggggccaccccacggatccccccgaatgccttggaattagagtacctgtacggtggcggatcgggatccggaagcggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggaattagatcactagtccagtgtggtggaattataacttcgtatagcatacattatacgaagttataattctgcagatatccagcacagtggcggccacctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtggtggagaaccgtatataagtgcagtagtcgccgttaacgttctttttcgcaacgggtttgccgccagaacacaggatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaatgaataaggccgctcgaataacttcgtatagcatacattatacgaagttattcgagtctagagggcccgttagatctacagcttcctgccaacttgacactgcgtggggctagcaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtttccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttg

SEQ ID NO: 134 - DNA, 1353 bp, synthetic construct
  source 1..1353: mol_type = other DNA
ttatatatgttcacttcccggagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataatatggccacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggaattagatcactagtccagtgtggt

SEQ ID NO: 135 - DNA, 21 bp, synthetic construct
  source 1..21: mol_type = other DNA
aaggaagaaaacgaactccaa

SEQ ID NO: 136 - DNA, 21 bp, synthetic construct
  source 1..21: mol_type = other DNA
acaaagggaaaaatgaactaa

SEQ ID NO: 137 - DNA, 21 bp, synthetic construct
  source 1..21: mol_type = other DNA
cacagaaggtaaaatagatga

SEQ ID NO: 138 - DNA, 21 bp, synthetic construct
  source 1..21: mol_type = other DNA
ccccacaactgatcgtataca

SEQ ID NO: 139 - DNA, 21 bp, synthetic construct
  source 1..21: mol_type = other DNA
gaaactaaagtgatgcctgta

SEQ ID NO: 140 - DNA, 21 bp, synthetic construct
  source 1..21: mol_type = other DNA
caagatacttgcctggatctg

SEQ ID NO: 141 - DNA, 21 bp, synthetic construct
  source 1..21: mol_type = other DNA
aaacagagatcttataattgt

SEQ ID NO: 142 - DNA, 21 bp, synthetic construct
  source 1..21: mol_type = other DNA
ggtgaatacgtgtcctgtctt

SEQ ID NO: 143 - DNA, 54 bp, synthetic construct
  source 1..54: mol_type = other DNA
tcgtcggcagcgtcagatgtgtataagagacaggtgtttccaccattcatctca

SEQ ID NO: 144 - DNA, 55 bp, synthetic construct
  source 1..55: mol_type = other DNA
gtctcgtgggctcggagatgtgtataagagacaggtggaggaggtagtatacaga

SEQ ID NO: 145 - DNA, 54 bp, synthetic construct
  source 1..54: mol_type = other DNA
tcgtcggcagcgtcagatgtgtataagagacagtcggcttgctaataaagatcc

SEQ ID NO: 146 - DNA, 55 bp, synthetic construct
  source 1..55: mol_type = other DNA
gtctcgtgggctcggagatgtgtataagagacagtgtggtctggtctacactaac

SEQ ID NO: 147 - DNA, 55 bp, synthetic construct
  source 1..55: mol_type = other DNA
tcgtcggcagcgtcagatgtgtataagagacaggagtggtgaaaaagaagggatc

SEQ ID NO: 148 - DNA, 53 bp, synthetic construct
  source 1..53: mol_type = other DNA
gtctcgtgggctcggagatgtgtataagagacagagaagaagggttggcttga

SEQ ID NO: 149 - DNA, 57 bp, synthetic construct
  source 1..57: mol_type = other DNA
tcgtcggcagcgtcagatgtgtataagagacagcttctgtctgctctcattaaactg

SEQ ID NO: 150 - DNA, 54 bp, synthetic construct
  source 1..54: mol_type = other DNA
gtctcgtgggctcggagatgtgtataagagacagagacaaagccagagccaata

SEQ ID NO: 151 - DNA, 52 bp, synthetic construct
  source 1..52: mol_type = other DNA
tcgtcggcagcgtcagatgtgtataagagacaggaagagcctccagtctctg

SEQ ID NO: 152 - DNA, 53 bp, synthetic construct
  source 1..53: mol_type = other DNA
gtctcgtgggctcggagatgtgtataagagacagctctaagcctgcctctgtg

SEQ ID NO: 153 - DNA, 53 bp, synthetic construct
  source 1..53: mol_type = other DNA
tcgtcggcagcgtcagatgtgtataagagacagccggagaaaagagagttgca

SEQ ID NO: 154 - DNA, 55 bp, synthetic construct
  source 1..55: mol_type = other DNA
gtctcgtgggctcggagatgtgtataagagacagactacagaccgtcacattagc

SEQ ID NO: 155 - DNA, 55 bp, synthetic construct
  source 1..55: mol_type = other DNA
tcgtcggcagcgtcagatgtgtataagagacaggatacatggaatgagggaaggg

SEQ ID NO: 156 - DNA, 58 bp, synthetic construct
  source 1..58: mol_type = other DNA
gtctcgtgggctcggagatgtgtataagagacagctctctccatcctctgaaatgttc

SEQ ID NO: 157 - DNA, 54 bp, synthetic construct
  source 1..54: mol_type = other DNA
tcgtcggcagcgtcagatgtgtataagagacagtgagaggcaaagatgctttga

SEQ ID NO: 158 - DNA, 60 bp, synthetic construct
  source 1..60: mol_type = other DNA
gtctcgtgggctcggagatgtgtataagagacagttctttctactttttaaatgtggcca

SEQ ID NO: 159 - DNA, 59 bp, synthetic construct
  source 1..59: mol_type = other DNA
tcgtcggcagcgtcagatgtgtataagagacagcccacatgatcaaatgataggaaatg

SEQ ID NO: 160 - DNA, 54 bp, synthetic construct
  source 1..54: mol_type = other DNA
gtctcgtgggctcggagatgtgtataagagacaggctccagccgatatttcaga

SEQ ID NO: 161 - DNA, 55 bp, synthetic construct
  source 1..55: mol_type = other DNA
tcgtcggcagcgtcagatgtgtataagagacagtccaggcaagtatcttgttcag

SEQ ID NO: 162 - DNA, 59 bp, synthetic construct
  source 1..59: mol_type = other DNA
gtctcgtgggctcggagatgtgtataagagacagctagctgtgtaatttccagatatgc

SEQ ID NO: 163 - DNA, 56 bp, synthetic construct
  source 1..56: mol_type = other DNA
tcgtcggcagcgtcagatgtgtataagagacagttttttgtagagacagggtctcg

SEQ ID NO: 164 - DNA, 56 bp, synthetic construct
  source 1..56: mol_type = other DNA
gtctcgtgggctcggagatgtgtataagagacaggagcaagttgtacctgtctagc

SEQ ID NO: 165 - DNA, 52 bp, synthetic construct
  source 1..52: mol_type = other DNA
tcgtcggcagcgtcagatgtgtataagagacagaaggcttggatagagggca

SEQ ID NO: 166 - DNA, 56 bp, synthetic construct
  source 1..56: mol_type = other DNA
gtctcgtgggctcggagatgtgtataagagacagcaggcaatgtttctccaacaga

SEQ ID NO: 167 - DNA, 56 bp, synthetic construct
  source 1..56: mol_type = other DNA
tcgtcggcagcgtcagatgtgtataagagacaggttgaaggtactcagatgctgag

SEQ ID NO: 168 - DNA, 57 bp, synthetic construct
  source 1..57: mol_type = other DNA
gtctcgtgggctcggagatgtgtataagagacagatagctgtatcttaccgcatgtt

SEQ ID NO: 169 - DNA, 56 bp, synthetic construct
  source 1..56: mol_type = other DNA
tcgtcggcagcgtcagatgtgtataagagacagtgaagtagcatttctaccctgtt

SEQ ID NO: 170 - DNA, 58 bp, synthetic construct
  source 1..58: mol_type = other DNA
gtctcgtgggctcggagatgtgtataagagacagattggtcaagatcagcatgtaaaa

SEQ ID NO: 171 - DNA, 54 bp, synthetic construct
  source 1..54: mol_type = other DNA
tcgtcggcagcgtcagatgtgtataagagacagtggctgatccgtactaatcct

SEQ ID NO: 172 - DNA, 59 bp, synthetic construct
  source 1..59: mol_type = other DNA
gtctcgtgggctcggagatgtgtataagagacagccagccgtatgttttctaataagca

SEQ ID NO: 173 - DNA, 53 bp, synthetic construct
  source 1..53: mol_type = other DNA
tcgtcggcagcgtcagatgtgtataagagacagtaggggtacagactgctggg

SEQ ID NO: 174 - DNA, 54 bp, synthetic construct
  source 1..54: mol_type = other DNA
gtctcgtgggctcggagatgtgtataagagacagctgcagtggtctggaaccaa

SEQ ID NO: 175 - DNA, 55 bp, synthetic construct
  source 1..55: mol_type = other DNA
tcgtcggcagcgtcagatgtgtataagagacagagaggatcgaggctcttccagg

SEQ ID NO: 176 - DNA, 54 bp, synthetic construct
  source 1..54: mol_type = other DNA
gtctcgtgggctcggagatgtgtataagagacagacaaccagagggggtaacgc

SEQ ID NO: 177 - DNA, 53 bp, synthetic construct
  source 1..53: mol_type = other DNA
tcgtcggcagcgtcagatgtgtataagagacagctgagtctgcacagagcggg

SEQ ID NO: 178 - DNA, 57 bp, synthetic construct
  source 1..57: mol_type = other DNA
gtctcgtgggctcggagatgtgtataagagacagcacttaggcagacatcctgtgct

SEQ ID NO: 179 - DNA, 50 bp, synthetic construct
  source 1..50: mol_type = other DNA
tcgtcggcagcgtcagatgtgtataagagacaggggattacaggcgcgcg

SEQ ID NO: 180 - DNA, 55 bp, synthetic construct
  source 1..55: mol_type = other DNA
gtctcgtgggctcggagatgtgtataagagacagacaagtcaaccaaacgctgtc

SEQ ID NO: 181 - DNA, 57 bp, synthetic construct
  source 1..57: mol_type = other DNA
tcgtcggcagcgtcagatgtgtataagagacagaaagatgggattttgtcatgttgc

SEQ ID NO: 182 - DNA, 51 bp, synthetic construct
  source 1..51: mol_type = other DNA
gtctcgtgggctcggagatgtgtataagagacagggcaggcaatgctgtct

SEQ ID NO: 183 - DNA, 53 bp, synthetic construct
  source 1..53: mol_type = other DNA
tcgtcggcagcgtcagatgtgtataagagacagatgtttctcggcaaccttgg

SEQ ID NO: 184 - DNA, 55 bp, synthetic construct
  source 1..55: mol_type = other DNA
gtctcgtgggctcggagatgtgtataagagacagcgggaaaactgtcctcggtat

SEQ ID NO: 185 - DNA, 59 bp, synthetic construct
  source 1..59: mol_type = other DNA
tcgtcggcagcgtcagatgtgtataagagacagcgtgtagtgtttttgtaactgatcgt

SEQ ID NO: 186 - DNA, 52 bp, synthetic construct
  source 1..52: mol_type = other DNA
gtctcgtgggctcggagatgtgtataagagacagtaggctgagggtcgctga

SEQ ID NO: 187 - DNA, 51 bp, synthetic construct
  source 1..51: mol_type = other DNA
tcgtcggcagcgtcagatgtgtataagagacagtgtcggggcgctcatagt

SEQ ID NO: 188 - DNA, 53 bp, synthetic construct
  source 1..53: mol_type = other DNA
gtctcgtgggctcggagatgtgtataagagacagccctggttaagcccctctg

SEQ ID NO: 189 - DNA, 54 bp, synthetic construct
  source 1..54: mol_type = other DNA
tcgtcggcagcgtcagatgtgtataagagacagaagtcagaccagctctttccc

SEQ ID NO: 190 - DNA, 53 bp, synthetic construct
  source 1..53: mol_type = other DNA
gtctcgtgggctcggagatgtgtataagagacaggcctgttgtaagctccacc

SEQ ID NO: 191 - DNA, 58 bp, synthetic construct
  source 1..58: mol_type = other DNA
tcgtcggcagcgtcagatgtgtataagagacagccctgcaacttaggaagaaaagaga

SEQ ID NO: 192 - DNA, 55 bp, synthetic construct
  source 1..55: mol_type = other DNA
gtctcgtgggctcggagatgtgtataagagacagcagccttcacttcagggtggt

SEQ ID NO: 193 - DNA, 54 bp, synthetic construct
  source 1..54: mol_type = other DNA
tcgtcggcagcgtcagatgtgtataagagacagtgactgacgctgatcgcacat

SEQ ID NO: 194 - DNA, 55 bp, synthetic construct
  source 1..55: mol_type = other DNA
gtctcgtgggctcggagatgtgtataagagacagcctccccaggttcagaggaca

SEQ ID NO: 195 - DNA, 53 bp, synthetic construct
  source 1..53: mol_type = other DNA
tcgtcggcagcgtcagatgtgtataagagacagtaggaccaaggtgcgtcagg

SEQ ID NO: 196 - DNA, 53 bp, synthetic construct
  source 1..53: mol_type = other DNA
gtctcgtgggctcggagatgtgtataagagacaggccgccaccggctttattc

SEQ ID NO: 197 - DNA, 51 bp, synthetic construct
  source 1..51: mol_type = other DNA
tcgtcggcagcgtcagatgtgtataagagacagttggctttgctggggcta

SEQ ID NO: 198 - DNA, 58 bp, synthetic construct
  source 1..58: mol_type = other DNA
gtctcgtgggctcggagatgtgtataagagacagcccatgggtctaacattcacagaa

SEQ ID NO: 199 - DNA, 54 bp, synthetic construct
  source 1..54: mol_type = other DNA
tcgtcggcagcgtcagatgtgtataagagacagcctctagggaggtttctgtga

SEQ ID NO: 200 - DNA, 55 bp, synthetic construct
  source 1..55: mol_type = other DNA
gtctcgtgggctcggagatgtgtataagagacaggggatgctgaagcaaagggta

SEQ ID NO: 201 - DNA, 62 bp, synthetic construct
  source 1..62: mol_type = other DNA
tcgtcggcagcgtcagatgtgtataagagacagggttgatgtgtgttattatttgtaattat

SEQ ID NO: 202 - DNA, 62 bp, synthetic construct
  source 1..62: mol_type = other DNA
gtctcgtgggctcggagatgtgtataagagacagctaacaacttcaactggatatccttata

SEQ ID NO: 203 - DNA, 58 bp, synthetic construct
  source 1..58: mol_type = other DNA
tcgtcggcagcgtcagatgtgtataagagacagcgcaacagaaaaagtatttaagcag

SEQ ID NO: 204 - DNA, 59 bp, synthetic construct
  source 1..59: mol_type = other DNA
gtctcgtgggctcggagatgtgtataagagacagttgctagacgctgaagactaatttt

SEQ ID NO: 205 - DNA, 56 bp, synthetic construct
  source 1..56: mol_type = other DNA
tcgtcggcagcgtcagatgtgtataagagacagctgagaatatctagcagcaacat

SEQ ID NO: 206 - DNA, 56 bp, synthetic construct
  source 1..56: mol_type = other DNA
gtctcgtgggctcggagatgtgtataagagacaggtctgtttgatctcaccatctt

SEQ ID NO: 207 - DNA, 58 bp, synthetic construct
  source 1..58: mol_type = other DNA
tcgtcggcagcgtcagatgtgtataagagacagcgcaaagcttcttcttgatctaaac

SEQ ID NO: 208 - DNA, 60 bp, synthetic construct
  source 1..60: mol_type = other DNA
gtctcgtgggctcggagatgtgtataagagacaggttgacattctctttgaagatatggt

SEQ ID NO: 209 - DNA, 59 bp, synthetic construct
  source 1..59: mol_type = other DNA
tcgtcggcagcgtcagatgtgtataagagacagccaatattttccataacttaaggtgc

SEQ ID NO: 210 - DNA, 62 bp, synthetic construct
  source 1..62: mol_type = other DNA
gtctcgtgggctcggagatgtgtataagagacaggcatctaacaaagatacttacatttgaa

SEQ ID NO: 211 - DNA, 53 bp, synthetic construct
  source 1..53: mol_type = other DNA
tcgtcggcagcgtcagatgtgtataagagacagtattaactctgggctgctgt

SEQ ID NO: 212 - DNA, 55 bp, synthetic construct
  source 1..55: mol_type = other DNA
gtctcgtgggctcggagatgtgtataagagacaggacataggagcagagctgaag

SEQ ID NO: 213 - DNA, 56 bp, synthetic construct
  source 1..56: mol_type = other DNA
tcgtcggcagcgtcagatgtgtataagagacaggcagaacccactaatacaaagga

SEQ ID NO: 214 - DNA, 58 bp, synthetic construct
  source 1..58: mol_type = other DNA
gtctcgtgggctcggagatgtgtataagagacagcacagacaagccctcagatatatt

SEQ ID NO: 215 - DNA, 57 bp, synthetic construct
  source 1..57: mol_type = other DNA
tcgtcggcagcgtcagatgtgtataagagacagtcattgtttttaccaaggatccat

SEQ ID NO: 216 - DNA, 57 bp, synthetic construct
  source 1..57: mol_type = other DNA
gtctcgtgggctcggagatgtgtataagagacagtaaatcataggctacagctgaaa

SEQ ID NO: 217 - DNA, 54 bp, synthetic construct; identical to SEQ ID NO: 171
  source 1..54: mol_type = other DNA

SEQ ID NO: 218 - DNA, 59 bp, synthetic construct; identical to SEQ ID NO: 172
  source 1..59: mol_type = other DNA

SEQ ID NO: 219 - DNA, 55 bp, synthetic construct
  source 1..55: mol_type = other DNA
tcgtcggcagcgtcagatgtgtataagagacagagctggcctctaagaccccctt

SEQ ID NO: 220 - DNA, 56 bp, synthetic construct
  source 1..56: mol_type = other DNA
gtctcgtgggctcggagatgtgtataagagacagcagctctgtctctcacctggcg

SEQ ID NO: 221 - DNA, 54 bp, synthetic construct
  source 1..54: mol_type = other DNA
tcgtcggcagcgtcagatgtgtataagagacagggagaggatttggctggtaac

SEQ ID NO: 222 - DNA, 54 bp, synthetic construct
  source 1..54: mol_type = other DNA
gtctcgtgggctcggagatgtgtataagagacagccctacctgaggtcacaagg

SEQ ID NO: 223 - DNA, 56 bp, synthetic construct
  source 1..56: mol_type = other DNA
tcgtcggcagcgtcagatgtgtataagagacagggctgtgatgtgccttatggaga

SEQ ID NO: 224 - DNA, 59 bp, synthetic construct
  source 1..59: mol_type = other DNA
gtctcgtgggctcggagatgtgtataagagacagacagaatactgaacccttgctgcca

SEQ ID NO: 225 - DNA, 57 bp, synthetic construct
  source 1..57: mol_type = other DNA
tcgtcggcagcgtcagatgtgtataagagacagtcttgagagagcattggtagtttg

SEQ ID NO: 226 - DNA, 59 bp, synthetic construct
  source 1..59: mol_type = other DNA
gtctcgtgggctcggagatgtgtataagagacagctctcatcttaatccctcacaatcc

SEQ ID NO: 227 - DNA, 54 bp, synthetic construct
  source 1..54: mol_type = other DNA
tcgtcggcagcgtcagatgtgtataagagacaggcacgattctcagccttcaag

SEQ ID NO: 228 - DNA, 53 bp, synthetic construct
  source 1..53: mol_type = other DNA
gtctcgtgggctcggagatgtgtataagagacagcccggccaaacctttttgg

SEQ ID NO: 229 - DNA, 74 bp, synthetic construct
  source 1..74: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
cagttgccatgtagaccccttgaagaggggaggggcctagggagccgcacttatatatgttcacttcccggagc

SEQ ID NO: 230 - DNA, 71 bp, synthetic construct
  source 1..71: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
gacaagtaactggttgagcacagggtactttattgatggtacatgacaagaccacactggactagtgatct

SEQ ID NO: 231 - DNA, 74 bp, synthetic construct
  source 1..74: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
gaggactttgattgcacattgttgtttttttaatagtcattccaaatatgttatatatgttcacttcccggagc

SEQ ID NO: 232 - DNA, 71 bp, synthetic construct
  source 1..71: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
aagtggggtggcttttaggatggcaagggacttcctgtaacaacgcatctaccacactggactagtgatct

SEQ ID NO: 233 - DNA, 74 bp, synthetic construct
  source 1..74: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
gtaatgttgactgtattttccaacttgttcaaattattaccagtgaatctttatatatgttcacttcccggagc

SEQ ID NO: 234 - DNA, 71 bp, synthetic construct
  source 1..71: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
taaatttttgggaatttattgatttgcatttaaaagggaactgctgacaaaccacactggactagtgatct

SEQ ID NO: 235 - DNA, 74 bp, synthetic construct
  source 1..74: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
aatatattgaacttttgtactgaatttttttgtaataagcaatcaaggttttatatatgttcacttcccggagc

SEQ ID NO: 236 - DNA, 71 bp, synthetic construct
  source 1..71: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
attaggttaatattgccttcttacaaaatttctattttaaaaaaaattataccacactggactagtgatct

SEQ ID NO: 237 - DNA, 74 bp, synthetic construct
  source 1..74: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
gggggtgtctaagtttccccttttaaggtttcaacaaatttcattgcactttatatatgttcacttcccggagc

SEQ ID NO: 238 - DNA, 71 bp, synthetic construct
  source 1..71: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
aaaaaaaaaaaaaacaccaattgggaatgcaacaactttattgaaaggaaaccacactggactagtgatct

SEQ ID NO: 239 - DNA, 74 bp, synthetic construct
  source 1..74: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
catactctgcttagaatttgggggaaaatttagaaatataattgacaggattatatatgttcacttcccggagc

SEQ ID NO: 240 - DNA, 71 bp, synthetic construct
  source 1..71: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
tgaatcttatatgacaaaatgtttcattcattataacaaatttccaataaaccacactggactagtgatct

SEQ ID NO: 241 - DNA, 53 bp, synthetic construct
  source 1..53: mol_type = other DNA
tcgtcggcagcgtcagatgtgtataagagacagaagggagaggtcaaggttgg

SEQ ID NO: 242 - DNA, 56 bp, synthetic construct
  source 1..56: mol_type = other DNA
gtctcgtgggctcggagatgtgtataagagacagtgcaattgccttaaacacacct

SEQ ID NO: 243 - DNA, 57 bp, synthetic construct
  source 1..57: mol_type = other DNA
tcgtcggcagcgtcagatgtgtataagagacaggtatttcctcagggaaggacgaag

SEQ ID NO: 244 - DNA, 53 bp, synthetic construct
  source 1..53: mol_type = other DNA
gtctcgtgggctcggagatgtgtataagagacagagtagctgggaccacagga

SEQ ID NO: 245 - DNA, 54 bp, synthetic construct
  source 1..54: mol_type = other DNA
tcgtcggcagcgtcagatgtgtataagagacagggcccagggagctcttaatac

SEQ ID NO: 246 - DNA, 55 bp, synthetic construct
  source 1..55: mol_type = other DNA
gtctcgtgggctcggagatgtgtataagagacagttcctgctgtaacgcttaggc

SEQ ID NO: 247 - DNA, 30 bp, synthetic construct
  source 1..30: mol_type = other DNA
cctattggtttaaaaatgagctgatttaac

SEQ ID NO: 248 - DNA, 18 bp, synthetic construct
  source 1..18: mol_type = other DNA
ccgaaatcggcaaaatcc

SEQ ID NO: 249 - DNA, 30 bp, synthetic construct
  source 1..30: mol_type = other DNA
cctattggttcaaaaatgagctgatttaac

SEQ ID NO: 250 - DNA, 18 bp, synthetic construct
  source 1..18: mol_type = other DNA
ccgaaatcggcaaaatcc

SEQ ID NO: 251 - DNA, 23 bp, synthetic construct
  source 1..23: mol_type = other DNA
cctattggttgaaaaatgagctg

SEQ ID NO: 252 - DNA, 18 bp, synthetic construct
  source 1..18: mol_type = other DNA
ccgaaatcggcaaaatcc

SEQ ID NO: 253 - DNA, 20 bp, synthetic construct
  source 1..20: mol_type = other DNA
ctgctgaagcctccgagatg

SEQ ID NO: 254 - DNA, 20 bp, synthetic construct
  source 1..20: mol_type = other DNA
aagcagcagaccttagcagg

SEQ ID NO: 255 - DNA, 18 bp, synthetic construct
  source 1..18: mol_type = other DNA
tgcttacctagtggagac

SEQ ID NO: 256 - DNA, 18 bp, synthetic construct
  source 1..18: mol_type = other DNA
tggtcactgcagaatgac

SEQ ID NO: 257 - DNA, 18 bp, synthetic construct
  source 1..18: mol_type = other DNA
acacagcaataccctatc

SEQ ID NO: 258 - DNA, 18 bp, synthetic construct
  source 1..18: mol_type = other DNA
ccagccgtatgttttcta

SEQ ID NO: 259 - skipped

SEQ ID NO: 260 - RNA, 18 nt, synthetic construct
  source 1..18: mol_type = other RNA
atttctactattgtagat

SEQ ID NO: 261 - RNA, 18 nt, synthetic construct
  source 1..18: mol_type = other RNA
atttctactgttgtagat

SEQ ID NO: 262 - RNA, 18 nt, synthetic construct
  source 1..18: mol_type = other RNA
atttctacttttgtagat

SEQ ID NO: 263 - RNA, 18 nt, synthetic construct
  source 1..18: mol_type = other RNA
atttctactagtgtagat

SEQ ID NO: 264 - RNA, 19 nt, synthetic construct
  source 1..19: mol_type = other RNA
atttctactgtgtgtagat

SEQ ID NO: 265 - RNA, 19 nt, synthetic construct
  source 1..19: mol_type = other RNA
  misc_difference 13: note = May be absent
atttctactdkktgtagat

SEQ ID NO: 266 - DNA, 27 bp, synthetic construct
  source 1..27: mol_type = other DNA
  misc_difference 4: note = Any nucleotide
  misc_difference 25: note = Any nucleotide
tttnggcaaataggaatggcaagangg

SEQ ID NO: 267 - DNA, 27 bp, synthetic construct
  source 1..27: mol_type = other DNA
  misc_difference 3: note = Any nucleotide
  misc_difference 24: note = Any nucleotide
ccntcttgccattcctatttgccnaaa

SEQ ID NO: 268 - RNA, 21 nt, synthetic construct
  source 1..21: mol_type = other RNA
ttggagttcgttttcttcctt

SEQ ID NO: 269 - RNA, 21 nt, synthetic construct
  source 1..21: mol_type = other RNA
  modified_base 5: mod_base = OTHER; note = 2-aminoadenine
ttggngttcgttttcttcctt

SEQ ID NO: 270 - DNA, 21 bp, synthetic construct
  source 1..21: mol_type = other DNA
aacctcaagcaaaagaaggaa

SEQ ID NO: 271 - RNA, 21 nt, synthetic construct
  source 1..21: mol_type = other RNA
ttagttcatttttccctttgt

SEQ ID NO: 272 - RNA, 21 nt, synthetic construct
  source 1..21: mol_type = other RNA
  modified_base 3: mod_base = OTHER; note = 2-aminoadenine
  modified_base 8: mod_base = OTHER; note = 2-aminoadenine
ttngttcntttttccctttgt

SEQ ID NO: 273 - DNA, 21 bp, synthetic construct
  source 1..21: mol_type = other DNA
aatcaagtaaaaagggaaaca

SEQ ID NO: 274 - RNA, 21 nt, synthetic construct
  source 1..21: mol_type = other RNA
tcatctattttaccttctgtg

SEQ ID NO: 275 - RNA, 21 nt, synthetic construct
  source 1..21: mol_type = other RNA
  modified_base 3: mod_base = OTHER; note = 2-aminoadenine
  modified_base 7: mod_base = OTHER; note = 2-aminoadenine
  modified_base 12: mod_base = OTHER; note = 2-aminoadenine
tcntctnttttnccttctgtg

SEQ ID NO: 276 - DNA, 21 bp, synthetic construct
  source 1..21: mol_type = other DNA
agtagataaaatggaagacac

SEQ ID NO: 277 - RNA, 21 nt, synthetic construct
  source 1..21: mol_type = other RNA
tgtatacgatcagttgtgggg

SEQ ID NO: 278 - RNA, 21 nt, synthetic construct
  source 1..21: mol_type = other RNA
  modified_base 4: mod_base = OTHER; note = 2-aminoadenine
  modified_base 6: mod_base = OTHER; note = 2-aminoadenine
  modified_base 9: mod_base = OTHER; note = 2-aminoadenine
  modified_base 12: mod_base = OTHER; note = 2-aminoadenine
tgtntncgntcngttgtgggg

SEQ ID NO: 279 - DNA, 21 bp, synthetic construct
  source 1..21: mol_type = other DNA
acatatgctagtcaacacccc

SEQ ID NO: 280 - RNA, 21 nt, synthetic construct
  source 1..21: mol_type = other RNA
tacaggcatcactttagtttc

SEQ ID NO: 281 - RNA, 21 nt, synthetic construct
  source 1..21: mol_type = other RNA
  modified_base 2: mod_base = OTHER; note = 2-aminoadenine
  modified_base 4: mod_base = OTHER; note = 2-aminoadenine
  modified_base 8: mod_base = OTHER; note = 2-aminoadenine
  modified_base 11: mod_base = OTHER; note = 2-aminoadenine
  modified_base 16: mod_base = OTHER; note = 2-aminoadenine
tncnggcntcnctttngtttc

SEQ ID NO: 282 - DNA, 21 bp, synthetic construct
  source 1..21: mol_type = other DNA
atgtccgtagtgaaatcaaag

SEQ ID NO: 283 - RNA, 21 nt, synthetic construct
  source 1..21: mol_type = other RNA
cagatccaggcaagtatcttg

SEQ ID NO: 284 - RNA, 21 nt, synthetic construct
  source 1..21: mol_type = other RNA
  modified_base 2: mod_base = OTHER; note = 2-aminoadenine
  modified_base 4: mod_base = OTHER; note = 2-aminoadenine
  modified_base 8: mod_base = OTHER; note = 2-aminoadenine
  modified_base 12: mod_base = OTHER; note = 2-aminoadenine
  modified_base 13: mod_base = OTHER; note = 2-aminoadenine
  modified_base 16: mod_base = OTHER; note = 2-aminoadenine
cngntccnggcnngtntcttg

SEQ ID NO: 285 - DNA, 21 bp, synthetic construct
  source 1..21: mol_type = other DNA
gtctaggtccgttcatagaac

SEQ ID NO: 286 - RNA, 21 nt, synthetic construct
  source 1..21: mol_type = other RNA
acaattataagatctctgttt

SEQ ID NO: 287 - RNA, 21 nt, synthetic construct
  source 1..21: mol_type = other RNA
  modified_base 1: mod_base = OTHER; note = 2-aminoadenine
  modified_base 3: mod_base = OTHER; note = 2-aminoadenine
  modified_base 4: mod_base = OTHER; note = 2-aminoadenine
  modified_base 7: mod_base = OTHER; note = 2-aminoadenine
  modified_base 9: mod_base = OTHER; note = 2-aminoadenine
  modified_base 10: mod_base = OTHER; note = 2-aminoadenine
  modified_base 12: mod_base = OTHER; note = 2-aminoadenine
ncnnttntnngntctctgttt

SEQ ID NO: 288 - DNA, 21 bp, synthetic construct
  source 1..21: mol_type = other DNA
tgttaatattctagagacaaa

SEQ ID NO: 289 - RNA, 21 nt, synthetic construct
  source 1..21: mol_type = other RNA
aagacaggacacgtattcacc

SEQ ID NO: 290 - RNA, 21 nt, synthetic construct
  source 1..21: mol_type = other RNA
  modified_base 1: mod_base = OTHER; note = 2-aminoadenine
  modified_base 2: mod_base = OTHER; note = 2-aminoadenine
  modified_base 4: mod_base = OTHER; note = 2-aminoadenine
  modified_base 6: mod_base = OTHER; note = 2-aminoadenine
  modified_base 9: mod_base = OTHER; note = 2-aminoadenine
  modified_base 11: mod_base = OTHER; note = 2-aminoadenine
  modified_base 15: mod_base = OTHER; note = 2-aminoadenine
  modified_base 19: mod_base = OTHER; note = 2-aminoadenine
nngncnggncncgtnttcncc

SEQ ID NO: 291 - DNA, 21 bp, synthetic construct
  source 1..21: mol_type = other DNA
ttctgtcctgtgcataagtgg

SEQ ID NO: 292-319 - skipped (28 entries)

SEQ ID NO: 320 - DNA, 39 bp, synthetic construct
  source 1..39: mol_type = other DNA
aatgtatttagaaaaataaacaaataggggtcagtgtta

SEQ ID NO: 321 - DNA, 39 bp, synthetic construct
  source 1..39: mol_type = other DNA
taacactgacccctatttgtttatttttctaaatacatt

SEQ ID NO: 322 - DNA, 39 bp, synthetic construct; identical to SEQ ID NO: 320
  source 1..39: mol_type = other DNA

SEQ ID NO: 323 - DNA, 39 bp, synthetic construct; identical to SEQ ID NO: 321
  source 1..39: mol_type = other DNA

SEQ ID NO: 324 - DNA, 16 bp, synthetic construct
  source 1..16: mol_type = other DNA
taaggcgatctctccg

SEQ ID NO: 325 - DNA, 80 bp, synthetic construct
  source 1..80: mol_type = other DNA
gttctttccttatgtaggcccttagggcccgtttctgaatggcctatgtgtaaatacaaggacttaacacaaggctatgg

SEQ ID NO: 326 - DNA, 74 bp, synthetic construct
  source 1..74: mol_type = other DNA
gttctttccttatgtaggcccttagggcccgaatggcctatgtgtaaatacaaggacttaacacaaggctatgg

SEQ ID NO: 327 - DNA, 74 bp, synthetic construct
  source 1..74: mol_type = other DNA
gttctttccttatgtaggcccttagggcccgtatggcctatgtgtaaatacaaggacttaacacaaggctatgg

SEQ ID NO: 328 - DNA, 80 bp, synthetic construct; identical to SEQ ID NO: 325
  source 1..80: mol_type = other DNA

SEQ ID NO: 329 - DNA, 76 bp, synthetic construct
  source 1..76: mol_type = other DNA
gttctttccttatgtaggcccttagggcccgtgaatggcctatgtgtaaatacaaggacttaacacaaggctatgg

SEQ ID NO: 330 - DNA, 74 bp, synthetic construct
  source 1..74: mol_type = other DNA
gttctttccttatgtaggcccttagggcctgaatggcctatgtgtaaatacaaggacttaacacaaggctatgg

SEQ ID NO: 331 - DNA, 74 bp, synthetic construct
  source 1..74: mol_type = other DNA
gttctttccttatgtaggcccttagggtctgaatggcctatgtgtaaatacaaggacttaacacaaggctatgg

SEQ ID NO: 332 - DNA, 65 bp, synthetic construct
  source 1..65: mol_type = other DNA
gttctttccttatgtaggcccttagggcctatgtgtaaatacaaggacttaacacaaggctatgg

SEQ ID NO: 333 - DNA, 75 bp, synthetic construct
  source 1..75: mol_type = other DNA
gttctttccttatgtaggcccttagggctctgaatggcctatgtgtaaatacaaggacttaacacaaggctatgg

SEQ ID NO: 334 - DNA, 56 bp, synthetic construct
  source 1..56: mol_type = other DNA
gttctttccttatgtaggcctatgtgtaaatacaaggacttaacacaaggctatgg

SEQ ID NO: 335 - DNA, 75 bp, synthetic construct
  source 1..75: mol_type = other DNA
gttctttccttatgtaggcccttagggccctgaatggcctatgtgtaaatacaaggacttaacacaaggctatgg

SEQ ID NO: 336 - DNA, 16 bp, synthetic construct
  source 1..16: mol_type = other DNA
tcctgagctctctccg

SEQ ID NO: 337 - DNA, 80 bp, synthetic construct; identical to SEQ ID NO: 325
  source 1..80: mol_type = other DNA

SEQ ID NO: 338 - DNA, 74 bp, synthetic construct; identical to SEQ ID NO: 326
  source 1..74: mol_type = other DNA

SEQ ID NO: 339 - DNA, 74 bp, synthetic construct; identical to SEQ ID NO: 327
  source 1..74: mol_type = other DNA

SEQ ID NO: 340 - DNA, 74 bp, synthetic construct; identical to SEQ ID NO: 330
  source 1..74: mol_type = other DNA

SEQ ID NO: 341 - DNA, 76 bp, synthetic construct; identical to SEQ ID NO: 329
  source 1..76: mol_type = other DNA

SEQ ID NO: 342 - DNA, 56 bp, synthetic construct; identical to SEQ ID NO: 334
  source 1..56: mol_type = other DNA

SEQ ID NO: 343 - DNA, 74 bp, synthetic construct; identical to SEQ ID NO: 331
  source 1..74: mol_type = other DNA

SEQ ID NO: 344 - DNA, 75 bp, synthetic construct
  source 1..75: mol_type = other DNA
gttctttccttatgtaggcccttagggcccgtaatggcctatgtgtaaatacaaggacttaacacaaggctatgg

SEQ ID NO: 345 - DNA, 75 bp, synthetic construct
  source 1..75: mol_type = other DNA
gttctttccttatgtaggcccttagggcccggaatggcctatgtgtaaatacaaggacttaacacaaggctatgg

SEQ ID NO: 346 - DNA, 75 bp, synthetic construct; identical to SEQ ID NO: 333
  source 1..75: mol_type = other DNA

SEQ ID NO: 347 - DNA, 25 bp, synthetic construct
  source 1..25: mol_type = other DNA
  misc_difference 4: note = Any nucleotide
tttnctgatggtccatgtctgttac

SEQ ID NO: 348 - DNA, 25 bp, synthetic construct
  source 1..25: mol_type = other DNA
  misc_difference 4: note = Any nucleotide
tttnctgatggtccatgtctgttac

SEQ ID NO: 349 - DNA, 25 bp, synthetic construct
  source 1..25: mol_type = other DNA
  misc_difference 4: note = Any nucleotide
tttngctcagcaggcacctgcctca

SEQ ID NO: 350 - DNA, 25 bp, synthetic construct
  source 1..25: mol_type = other DNA
  misc_difference 4: note = Any nucleotide
tttngctcagcaggcacctgcctca

SEQ ID NO: 351 - DNA, 25 bp, synthetic construct
  source 1..25: mol_type = other DNA
  misc_difference 4: note = Any nucleotide
tttnggcggggtccagttccgggat

SEQ ID NO: 352 - DNA, 25 bp, synthetic construct
  source 1..25: mol_type = other DNA
  misc_difference 4: note = Any nucleotide
tttnggcggggtccagttccgggat

SEQ ID NO: 353 - DNA, 25 bp, synthetic construct
  source 1..25: mol_type = other DNA
  misc_difference 4: note = Any nucleotide
tttngtcggcatggccccattcgca

SEQ ID NO: 354 - DNA, 25 bp, synthetic construct
  source 1..25: mol_type = other DNA
  misc_difference 4: note = Any nucleotide
tttngtcggcatggccccattcgca

SEQ ID NO: 355 - DNA, 24 bp, synthetic construct
  source 1..24: mol_type = other DNA
  misc_difference 4: note = Any nucleotide
tttnggatgccactaaaagggaaa

SEQ ID NO: 356-365 - skipped (10 entries)

SEQ ID NO: 366 - DNA, 24 bp, synthetic construct
  source 1..24: mol_type = other DNA
  misc_difference 4: note = Any nucleotide
tttnggatgccactaaaagggaaa

SEQ ID NO: 367 - DNA, 24 bp, synthetic construct
  source 1..24: mol_type = other DNA
  misc_difference 4: note = Any nucleotide
tttngggtgatcagacccaacagc

SEQ ID NO: 368 - DNA, 24 bp, synthetic construct
  source 1..24: mol_type = other DNA
  misc_difference 4: note = Any nucleotide
tttngggtgatcagacccaacagc

SEQ ID NO: 369 - DNA, 24 bp, synthetic construct
  source 1..24: mol_type = other DNA
  misc_difference 4: note = Any nucleotide
tttnggcaaataggaatggcaaga

SEQ ID NO: 370 - DNA, 24 bp, synthetic construct
  source 1..24: mol_type = other DNA
  misc_difference 4: note = Any nucleotide
tttnggcaaataggaatggcaaga

SEQ ID NO: 371 - DNA, 23 bp, synthetic construct
  source 1..23: mol_type = other DNA
  misc_difference 3: note = Any nucleotide
ggnagaacggtaaggataaacgg

SEQ ID NO: 372 - DNA, 24 bp, synthetic construct
  source 1..24: mol_type = other DNA
  misc_difference 4: note = Any nucleotide
tttngagctgcttaagcatttcaa

SEQ ID NO: 373 - DNA, 24 bp, synthetic construct
  source 1..24: mol_type = other DNA
  misc_difference 4: note = Any nucleotide
tttngagctgcttaagcatttcaa

SEQ ID NO: 374 - DNA, 23 bp, synthetic construct
  source 1..23: mol_type = other DNA
  misc_difference 3: note = Any nucleotide
ggnaactttacgaattcgtcgag

SEQ ID NO: 375 - DNA, 24 bp, synthetic construct
  source 1..24: mol_type = other DNA
  misc_difference 4: note = Any nucleotide
tttnggcatgaattataatgctgt

SEQ ID NO: 376 - DNA, 24 bp, synthetic construct
  source 1..24: mol_type = other DNA
  misc_difference 4: note = Any nucleotide
tttnggcatgaattataatgctgt

SEQ ID NO: 377 - DNA, 23 bp, synthetic construct
  source 1..23: mol_type = other DNA
  misc_difference 3: note = Any nucleotide
ggntgtcgtaatattaagtacgg

SEQ ID NO: 378 - DNA, 24 bp, synthetic construct
  source 1..24: mol_type = other DNA
  misc_difference 4: note = Any nucleotide
tttngaatctcaaaaataaaagac

SEQ ID NO: 379 - DNA, 24 bp, synthetic construct
  source 1..24: mol_type = other DNA
  misc_difference 4: note = Any nucleotide
tttngaatctcaaaaataaaagac

SEQ ID NO: 380 - DNA, 23 bp, synthetic construct
  source 1..23: mol_type = other DNA
  misc_difference 3: note = Any nucleotide
ggncagaaaataaaaactctaag

SEQ ID NO: 381 - DNA, 24 bp, synthetic construct
  source 1..24: mol_type = other DNA
  misc_difference 4: note = Any nucleotide
tttngagaaaatatgggttgaggt

SEQ ID NO: 382 - DNA, 24 bp, synthetic construct
  source 1..24: mol_type = other DNA
  misc_difference 4: note = Any nucleotide
tttngagaaaatatgggttgaggt

SEQ ID NO: 383 - DNA, 23 bp, synthetic construct
  source 1..23: mol_type = other DNA
  misc_difference 3: note = Any nucleotide
ggntggagttgggtataaaagag

SEQ ID NO: 384 - RNA, 20 nt, synthetic construct
  source 1..20: mol_type = other RNA
atctataaaaagggggtggg

SEQ ID NO: 385 - RNA, 20 nt, synthetic construct
  source 1..20: mol_type = other RNA
ctgctgttcctgggcaaaac

SEQ ID NO: 386 - RNA, 20 nt, synthetic construct
  source 1..20: mol_type = other RNA
  modified_base 1: mod_base = OTHER; note = 2-aminoadenine
  modified_base 5: mod_base = OTHER; note = 2-aminoadenine
  modified_base 7: mod_base = OTHER; note = 2-aminoadenine
  modified_base 8: mod_base = OTHER; note = 2-aminoadenine
  modified_base 9: mod_base = OTHER; note = 2-aminoadenine
  modified_base 10: mod_base = OTHER; note = 2-aminoadenine
  modified_base 11: mod_base = OTHER; note = 2-aminoadenine
ntctntnnnnngggggtggg

SEQ ID NO: 387 - RNA, 20 nt, synthetic construct
  source 1..20: mol_type = other RNA
  modified_base 16: mod_base = OTHER; note = 2-aminoadenine
  modified_base 17: mod_base = OTHER; note = 2-aminoadenine
  modified_base 18: mod_base = OTHER; note = 2-aminoadenine
  modified_base 19: mod_base = OTHER; note = 2-aminoadenine
ctgctgttcctgggcnnnnc

SEQ ID NO: 388 - RNA, 20 nt, synthetic construct
  source 1..20: mol_type = other RNA
taagaatcagcgtgtggttg

SEQ ID NO: 389 - RNA, 20 nt, synthetic construct
  source 1..20: mol_type = other RNA
ttttgttttttaataagatg

SEQ ID NO: 390 - RNA, 20 nt, synthetic construct
  source 1..20: mol_type = other RNA
  modified_base 2: mod_base = OTHER; note = 2-aminoadenine
  modified_base 3: mod_base = OTHER; note = 2-aminoadenine
  modified_base 5: mod_base = OTHER; note = 2-aminoadenine
  modified_base 6: mod_base = OTHER; note = 2-aminoadenine
  modified_base 9: mod_base = OTHER; note = 2-aminoadenine
tnngnntcngcgtgtggttg

SEQ ID NO: 391 - RNA, 20 nt, synthetic construct
  source 1..20: mol_type = other RNA
  modified_base 12: mod_base = OTHER; note = 2-aminoadenine
  modified_base 13: mod_base = OTHER; note = 2-aminoadenine
  modified_base 15: mod_base = OTHER; note = 2-aminoadenine
  modified_base 16: mod_base = OTHER; note = 2-aminoadenine
  modified_base 18: mod_base = OTHER; note = 2-aminoadenine
ttttgttttttnntnngntg

SEQ ID NO: 392 - RNA, 20 nt, synthetic construct
  source 1..20: mol_type = other RNA
aaagacagttttgctctggg

SEQ ID NO: 393 - RNA, 20 nt, synthetic construct
  source 1..20: mol_type = other RNA
tgctttgttgtgaaactgaa

SEQ ID NO: 394 - RNA, 20 nt, synthetic construct
  source 1..20: mol_type = other RNA
  modified_base 1: mod_base = OTHER; note = 2-aminoadenine
  modified_base 2: mod_base = OTHER; note = 2-aminoadenine
  modified_base 3: mod_base = OTHER; note = 2-aminoadenine
  modified_base 5: mod_base = OTHER; note = 2-aminoadenine
  modified_base 7: mod_base = OTHER; note = 2-aminoadenine
nnngncngttttgctctggg

SEQ ID NO: 395 - RNA, 20 nt, synthetic construct
  source 1..20: mol_type = other RNA
  modified_base 13: mod_base = OTHER; note = 2-aminoadenine
  modified_base 14: mod_base = OTHER; note = 2-aminoadenine
  modified_base 15: mod_base = OTHER; note = 2-aminoadenine
  modified_base 19: mod_base = OTHER; note = 2-aminoadenine
  modified_base 20: mod_base = OTHER; note = 2-aminoadenine
tgctttgttgtgnnnctgnn

SEQ ID NO: 396 - RNA, 20 nt, synthetic construct
  source 1..20: mol_type = other RNA
ttaaaaagactgttttggct

SEQ ID NO: 397 - RNA, 20 nt, synthetic construct
  source 1..20: mol_type = other RNA
tctgctttttgtattaaacc

SEQ ID NO: 398 - RNA, 20 nt, synthetic construct
  source 1..20: mol_type = other RNA
  modified_base 3: mod_base = OTHER; note = 2-aminoadenine
  modified_base 4: mod_base = OTHER; note = 2-aminoadenine
  modified_base 5: mod_base = OTHER; note = 2-aminoadenine
  modified_base 6: mod_base = OTHER; note = 2-aminoadenine
  modified_base 7: mod_base = OTHER; note = 2-aminoadenine
  modified_base 9: mod_base = OTHER; note = 2-aminoadenine
ttnnnnngnctgttttggct

SEQ ID NO: 399 - RNA, 20 nt, synthetic construct
  source 1..20: mol_type = other RNA
  modified_base 13: mod_base = OTHER; note = 2-aminoadenine
  modified_base 16: mod_base = OTHER; note = 2-aminoadenine
  modified_base 17: mod_base = OTHER; note = 2-aminoadenine
  modified_base 18: mod_base = OTHER; note = 2-aminoadenine
tctgctttttgtnttnnncc

SEQ ID NO: 400 - RNA, 20 nt, synthetic construct
  source 1..20: mol_type = other RNA
taagaataatactgtgcttc

SEQ ID NO: 401 - RNA, 20 nt, synthetic construct
  source 1..20: mol_type = other RNA
tccgttctgggggaaaaaaa

SEQ ID NO: 402 - RNA, 20 nt, synthetic construct
  source 1..20: mol_type = other RNA
  modified_base 2: mod_base = OTHER; note = 2-aminoadenine
  modified_base 3: mod_base = OTHER; note = 2-aminoadenine
  modified_base 5: mod_base = OTHER; note = 2-aminoadenine
  modified_base 6: mod_base = OTHER; note = 2-aminoadenine
  modified_base 8: mod_base = OTHER; note = 2-aminoadenine
  modified_base 9: mod_base = OTHER; note = 2-aminoadenine
  modified_base 11: mod_base = OTHER; note = 2-aminoadenine
tnngnntnntnctgtgcttc

SEQ ID NO: 403 - RNA, 20 nt, synthetic construct
  source 1..20: mol_type = other RNA
  modified_base 14: mod_base = OTHER; note = 2-aminoadenine
  modified_base 15: mod_base = OTHER; note = 2-aminoadenine
  modified_base 16: mod_base = OTHER; note = 2-aminoadenine
  modified_base 17: mod_base = OTHER; note = 2-aminoadenine
  modified_base 18: mod_base = OTHER; note = 2-aminoadenine
  modified_base 19: mod_base = OTHER; note = 2-aminoadenine
  modified_base 20: mod_base = OTHER; note = 2-aminoadenine
tccgttctgggggnnnnnnn

SEQ ID NO: 404 - RNA, 20 nt, synthetic construct
  source 1..20: mol_type = other RNA
cagtaagcaaactggcttcc

SEQ ID NO: 405 - RNA, 20 nt, synthetic construct
  source 1..20: mol_type = other RNA
ctgttcctctcaaacaaagg

SEQ ID NO: 406 - RNA, 20 nt, synthetic construct
  source 1..20: mol_type = other RNA
  modified_base 2: mod_base = OTHER; note = 2-aminoadenine
  modified_base 5: mod_base = OTHER; note = 2-aminoadenine
  modified_base 6: mod_base = OTHER; note = 2-aminoadenine
  modified_base 9: mod_base = OTHER; note = 2-aminoadenine
  modified_base 10: mod_base = OTHER; note = 2-aminoadenine
  modified_base 11: mod_base = OTHER; note = 2-aminoadenine
cngtnngcnnnctggcttcc

SEQ ID NO: 407 - RNA, 20 nt, synthetic construct
  source 1..20: mol_type = other RNA
  modified_base 12: mod_base = OTHER; note = 2-aminoadenine
  modified_base 13: mod_base = OTHER; note = 2-aminoadenine
  modified_base 14: mod_base = OTHER; note = 2-aminoadenine
  modified_base 16: mod_base = OTHER; note = 2-aminoadenine
  modified_base 17: mod_base = OTHER; note = 2-aminoadenine
  modified_base 18: mod_base = OTHER; note = 2-aminoadenine
ctgttcctctcnnncnnngg

SEQ ID NO: 408 - RNA, 20 nt, synthetic construct
  source 1..20: mol_type = other RNA
ccataaataaatgctgcctg

SEQ ID NO: 409 - RNA, 20 nt, synthetic construct
  source 1..20: mol_type = other RNA
tcttccttgcacccaaacag

SEQ ID NO: 410 - RNA, 20 nt, synthetic construct
  source 1..20: mol_type = other RNA
  modified_base 3: mod_base = OTHER; note = 2-aminoadenine
  modified_base 5: mod_base = OTHER; note = 2-aminoadenine
  modified_base 6: mod_base = OTHER; note = 2-aminoadenine
  modified_base 7: mod_base = OTHER; note = 2-aminoadenine
  modified_base 9: mod_base = OTHER; note = 2-aminoadenine
  modified_base 10: mod_base = OTHER; note = 2-aminoadenine
  modified_base 11: mod_base = OTHER; note = 2-aminoadenine
ccntnnntnnntgctgcctg

SEQ ID NO: 411 - RNA, 20 nt, synthetic construct
  source 1..20: mol_type = other RNA
  modified_base 11: mod_base = OTHER; note = 2-aminoadenine
  modified_base 15: mod_base = OTHER; note = 2-aminoadenine
  modified_base 16: mod_base = OTHER; note = 2-aminoadenine
  modified_base 17: mod_base = OTHER; note = 2-aminoadenine
  modified_base 19: mod_base = OTHER; note = 2-aminoadenine
tcttccttgcncccnnncng

SEQ ID NO: 412 - DNA, 22 bp, synthetic construct
  source 1..22: mol_type = other DNA
aatgtatttagaaaaataaaca

SEQ ID NO: 413 - DNA, 22 bp, synthetic construct
  source 1..22: mol_type = other DNA
attgtgactggggataaacaaa

SEQ ID NO: 414 - DNA, 25 bp, synthetic construct
  source 1..25: mol_type = other DNA
tttcctgatggtccatgtctgttac

SEQ ID NO: 415 - DNA, 25 bp, synthetic construct
  source 1..25: mol_type = other DNA
tttcctgatggtccatgtctgaatt

SEQ ID NO: 416 - DNA, 24 bp, synthetic construct
  source 1..24: mol_type = other DNA
tttcctgatggtccatgtctgaat

SEQ ID NO: 417 - DNA, 25 bp, synthetic construct
  source 1..25: mol_type = other DNA
tttggctcagcaggcacctgcctca

SEQ ID NO: 418 - DNA, 25 bp, synthetic construct
  source 1..25: mol_type = other DNA
cttagctcagcaggcacctgcccat

SEQ ID NO: 419 - DNA, 24 bp, synthetic construct
  source 1..24: mol_type = other DNA
cttagctcagcaggcacctgccca

SEQ ID NO: 420 - DNA, 25 bp, synthetic construct
  source 1..25: mol_type = other DNA
tttgggcggggtccagttccgggat

SEQ ID NO: 421 - DNA, 25 bp, synthetic construct
  source 1..25: mol_type = other DNA
tttggtcggcatggccccattcgca

SEQ ID NO: 422 - DNA, 24 bp, synthetic construct
  source 1..24: mol_type = other DNA
tttaggatgccactaaaagggaaa

SEQ ID NO: 423 - DNA, 24 bp, synthetic construct
  source 1..24: mol_type = other DNA
tttggggtgatcagacccaacacc

SEQ ID NO: 424 - DNA, 24 bp, synthetic construct
  source 1..24: mol_type = other DNA
tttggggtgatcagacccaacagc

SEQ ID NO: 425 - DNA, 24 bp, synthetic construct
  source 1..24: mol_type = other DNA
tttggggtgatcagacccaacccc

SEQ ID NO: 426 - DNA, 24 bp, synthetic construct
  source 1..24: mol_type = other DNA
gttggggtgatcagacccaacacc

SEQ ID NO: 427 - DNA, 24 bp, synthetic construct
  source 1..24: mol_type = other DNA
gttagggtgatcagacccaacact

SEQ ID NO: 428 - DNA, 23 bp, synthetic construct
  source 1..23: mol_type = other DNA
tktagggtgatcagacccaacac

SEQ ID NO: 429 - DNA, 23 bp, synthetic construct
  source 1..23: mol_type = other DNA
ttggggtgatcagacccaacacc

SEQ ID NO: 430 - DNA, 24 bp, synthetic construct
  source 1..24: mol_type = other DNA
attggggtgatcagacccaacacc

SEQ ID NO: 431 - DNA, 24 bp, synthetic construct
  source 1..24: mol_type = other DNA
gttggggtgatcagacccaacatc

SEQ ID NO: 432 - DNA, 24 bp, synthetic construct
  source 1..24: mol_type = other DNA
gttggggtgatcagacccaacgcc

SEQ ID NO: 433 - DNA, 23 bp, synthetic construct
  source 1..23: mol_type = other DNA
tktggggtgatcagacccaacgc

SEQ ID NO: 434 - DNA, 24 bp, synthetic construct
  source 1..24: mol_type = other DNA
gttggggtgatcagacccaacact

SEQ ID NO: 435 - DNA, 23 bp, synthetic construct
  source 1..23: mol_type = other DNA
tktggggtgatcagacccaacac

SEQ ID NO: 436 - DNA, 24 bp, synthetic construct
  source 1..24: mol_type = other DNA
tttggggtgatcaggcccaacacc

SEQ ID NO: 437 - DNA, 24 bp, synthetic construct
  source 1..24: mol_type = other DNA
gttggggtgatcagacccaacagc

SEQ ID NO: 438 - DNA, 24 bp, synthetic construct
  source 1..24: mol_type = other DNA
gttcgggtgatcagacccaacacc

SEQ ID NO: 439 - DNA, 24 bp, synthetic construct
  source 1..24: mol_type = other DNA
gttggggtgatcagacccaacaca

SEQ ID NO: 440 - DNA, 23 bp, synthetic construct
  source 1..23: mol_type = other DNA
ttggggtgatcagacccaacacg

SEQ ID NO: 441 - DNA, 23 bp, synthetic construct
  source 1..23: mol_type = other DNA
tktagggtgatcagacccaacac

SEQ ID NO: 442 - DNA, 23 bp, synthetic construct
  source 1..23: mol_type = other DNA
ttggggtgatcagacccaacact

SEQ ID NO: 443 - DNA, 23 bp, synthetic construct
  source 1..23: mol_type = other DNA
tktggggtgatcagacccaacgc

SEQ ID NO: 444 - DNA, 24 bp, synthetic construct
  source 1..24: mol_type = other DNA
gttggggtgatcagacccaactcc

SEQ ID NO: 445 - DNA, 24 bp, synthetic construct
  source 1..24: mol_type = other DNA
gttggggtgatcagacccaacacg

SEQ ID NO: 446 - DNA, 24 bp, synthetic construct
  source 1..24: mol_type = other DNA
gtttgggtgatcagacccaacacc

SEQ ID NO: 447 - DNA, 24 bp, synthetic construct
  source 1..24: mol_type = other DNA
tttggggtgatcagacctaacact

SEQ ID NO: 448 - DNA, 23 bp, synthetic construct
  source 1..23: mol_type = other DNA
tttggggtgatcagacctaacac

SEQ ID NO: 449 - DNA, 24 bp, synthetic construct
  source 1..24: mol_type = other DNA
cttggggtgatcagacccaacatc

SEQ ID NO: 450 - DNA, 24 bp, synthetic construct
  source 1..24: mol_type = other DNA
gttagggtgaccagacccaacacc

SEQ ID NO: 451 - DNA, 24 bp, synthetic construct
  source 1..24: mol_type = other DNA
gttggagtgatcagacccaacacc

SEQ ID NO: 452 - DNA, 24 bp, synthetic construct
  source 1..24: mol_type = other DNA
tttgggcaaataggaatggcaaga

SEQ ID NO: 453 - DNA, 24 bp, synthetic construct
  source 1..24: mol_type = other DNA
tttaggcaaataggaatggccata

SEQ ID NO: 454 - DNA, 23 bp, synthetic construct
  source 1..23: mol_type = other DNA
gggagaacggtaaggataaacgg

SEQ ID NO: 455 - DNA, 23 bp, synthetic construct
  source 1..23: mol_type = other DNA
gggagaacggtaaagataaacat

SEQ ID NO: 456 - DNA, 23 bp, synthetic construct
  source 1..23: mol_type = other DNA
gggagaacgggaaggacaaaagg

SEQ ID NO: 457 - DNA, 22 bp, synthetic construct
  source 1..22: mol_type = other DNA
ggtagaacagtaggataaacgg

SEQ ID NO: 458 - DNA, 23 bp, synthetic construct
  source 1..23: mol_type = other DNA
gagagaacggtaaggataaccac

SEQ ID NO: 459 - DNA, 23 bp, synthetic construct
  source 1..23: mol_type = other DNA
tggaaaaacggaaggataaacgg

SEQ ID NO: 460 - DNA, 22 bp, synthetic construct
  source 1..22: mol_type = other DNA
ggaaaaacggaaggataaacgg

SEQ ID NO: 461 - DNA, 23 bp, synthetic construct
  source 1..23: mol_type = other DNA
ggaagaacggaaggatataaggg

SEQ ID NO: 462 - DNA, 22 bp, synthetic construct
  source 1..22: mol_type = other DNA
ggaagaacggaaggatwaaggg

SEQ ID NO: 463 - DNA, 23 bp, synthetic construct
  source 1..23: mol_type = other DNA
ggaagaagggtaaggataaatgg

SEQ ID NO: 464 - DNA, 23 bp, synthetic construct
  source 1..23: mol_type = other DNA
ggaagaaaggtaaggatgaagga

SEQ ID NO: 465 - DNA, 22 bp, synthetic construct
  source 1..22: mol_type = other DNA
ggaagaaaggtaaggatgaagg

SEQ ID NO: 466 - DNA, 23 bp, synthetic construct
  source 1..23: mol_type = other DNA
ggttgaacggtaagataaacggg

SEQ ID NO: 467 - DNA, 22 bp, synthetic construct
  source 1..22: mol_type = other DNA
ggttgaacggtaagataaacgg

SEQ ID NO: 468 - DNA, 23 bp, synthetic construct
  source 1..23: mol_type = other DNA
ggaagaaagataaggataaacga

SEQ ID NO: 469 - DNA, 23 bp, synthetic construct
  source 1..23: mol_type = other DNA
gggagaacggtaagataaacaga

SEQ ID NO: 470 - DNA, 22 bp, synthetic construct
  source 1..22: mol_type = other DNA
gggagaacggtaagataaacag

SEQ ID NO: 471 - DNA, 23 bp, synthetic construct
  source 1..23: mol_type = other DNA
aggagaaaggtaaggataatcgg

SEQ ID NO: 472 - DNA, 23 bp, synthetic construct
  source 1..23: mol_type = other DNA
acggcagaggaaaggataaacgg

SEQ ID NO: 473 - DNA, 23 bp, synthetic construct
  source 1..23: mol_type = other DNA
gaaagaacggtaagagataaacg

SEQ ID NO: 474 - DNA, 23 bp, synthetic construct
  source 1..23: mol_type = other DNA
gaaagaacggtaargataaacgg

SEQ ID NO: 475 - DNA, 24 bp, synthetic construct
  source 1..24: mol_type = other DNA
tttggagctgcttaagcatttcaa

SEQ ID NO: 476 - DNA, 23 bp, synthetic construct
  source 1..23: mol_type = other DNA
gggaactttacgaattcgtcgag

SEQ ID NO: 477 - DNA, 23 bp, synthetic construct
  source 1..23: mol_type = other DNA
gggaacttttcgaatttgtagag

SEQ ID NO: 478 - DNA, 23 bp, synthetic construct
  source 1..23: mol_type = other DNA
ggaaactttacgtcttcgtcgaa

SEQ ID NO: 479 - DNA, 23 bp, synthetic construct
  source 1..23: mol_type = other DNA
ggtaactttacgaattcggtcaa

SEQ ID NO: 480 - DNA, 23 bp, synthetic construct
  source 1..23: mol_type = other DNA
ggtaactttacgaattcstcaac

SEQ ID NO: 481 - DNA, 23 bp, synthetic construct
  source 1..23: mol_type = other DNA
ggtaactttacgaattgtcgaag

SEQ ID NO: 482 - DNA, 22 bp, synthetic construct
  source 1..22: mol_type = other DNA
ggtaactttacgaattgtcrag

SEQ ID NO: 483 - DNA, 23 bp, synthetic construct
  source 1..23: mol_type = other DNA
gggaactttactaatccgtcaaa

SEQ ID NO: 484 - DNA, 23 bp, synthetic construct
  source 1..23: mol_type = other DNA
gggaaatttacgaattcgtcctg

SEQ ID NO: 485 - DNA, 23 bp, synthetic construct
  source 1..23: mol_type = other DNA
gggatctttacgaactcgtagaa

SEQ ID NO: 486 - DNA, 23 bp, synthetic construct
  source 1..23: mol_type = other DNA
ggtaactttacgaaaacgtagaa

SEQ ID NO: 487 - DNA, 23 bp, synthetic construct
  source 1..23: mol_type = other DNA
gggaactttacgaagacgtagag

SEQ ID NO: 488 - DNA, 23 bp, synthetic construct
  source 1..23: mol_type = other DNA
ggtaactttacggaaacgtcgaa

SEQ ID NO: 489 - DNA, 23 bp, synthetic construct
  source 1..23: mol_type = other DNA
ggtaactttccgaattcgtctct

SEQ ID NO: 490 - DNA, 22 bp, synthetic construct
  source 1..22: mol_type = other DNA
ggtaacttttcgaatcatcgag

SEQ ID NO: 491 - DNA, 24 bp, synthetic construct
  source 1..24: mol_type = other DNA
tttaggcatgaattataatgctgt

SEQ ID NO: 492 - DNA, 23 bp, synthetic construct
  source 1..23: mol_type = other DNA
ttaggcatgaattatagtgctgt

SEQ ID NO: 493 - DNA, 24 bp, synthetic construct
  source 1..24: mol_type = other DNA
tttaggcatgagttataatgctgt

SEQ ID NO: 494 - DNA, 24 bp, synthetic construct
  source 1..24: mol_type = other DNA
tttaggcatgaattatagtgctgt

SEQ ID NO: 495 - DNA, 23 bp, synthetic construct
  source 1..23: mol_type = other DNA
ggttgtcgtaatattaagtacgg

SEQ ID NO: 496 - DNA, 23 bp, synthetic construct
  source 1..23: mol_type = other DNA
gggtgtcgtaatataagtacggt

SEQ ID NO: 497 - DNA, 22 bp, synthetic construct
  source 1..22: mol_type = other DNA
gggtgtcgtaatataagtacgg

SEQ ID NO: 498 - DNA, 23 bp, synthetic construct
  source 1..23: mol_type = other DNA
ggttgtcgtaatattaagtgcgg

SEQ ID NO: 499 - DNA, 23 bp, synthetic construct
  source 1..23: mol_type = other DNA
ggttgtcgtaatattaagtacag

SEQ ID NO: 500 - DNA, 23 bp, synthetic construct
  source 1..23: mol_type = other DNA
ggttgtcgtaatattaagtattt

SEQ ID NO: 501 - DNA, 23 bp, synthetic construct
  source 1..23: mol_type = other DNA
ggctgtcgtaatattgagtacgg

SEQ ID NO: 502 - DNA, 23 bp, synthetic construct
  source 1..23: mol_type = other DNA
ggttgccgtaatattaagtacgg

SEQ ID NO: 503 - DNA, 23 bp, synthetic construct
  source 1..23: mol_type = other DNA
ggttgtcgtaatattgagtacgg

SEQ ID NO: 504 - DNA, 23 bp, synthetic construct
  source 1..23: mol_type = other DNA
ggttgtcgtaatattaaatgcgg

SEQ ID NO: 505 - DNA, 23 bp, synthetic construct
  source 1..23: mol_type = other DNA
ggttgtcgtgatattaagtacgg

SEQ ID NO: 506 - DNA, 23 bp, synthetic construct
  source 1..23: mol_type = other DNA
ggttgtcgtaatattgggtacgg

SEQ ID NO: 507 - DNA, 24 bp, synthetic construct
  source 1..24: mol_type = other DNA
tttagaatctcaaaaataaaagac

SEQ ID NO: 508 - DNA, 24 bp, synthetic construct
  source 1..24: mol_type = other DNA
tttagaatctcaaaaataaaaaca

SEQ ID NO: 509 - DNA, 23 bp, synthetic construct
  source 1..23: mol_type = other DNA
tttagaatctcaaaaataaaaac

SEQ ID NO: 510 - DNA, 24 bp, synthetic construct
  source 1..24: mol_type = other DNA
tttagaatctcaaaactaaaagga

SEQ ID NO: 511 - DNA, 23 bp, synthetic construct
  source 1..23: mol_type = other DNA
ggacagaaaataaaaactctaag

SEQ ID NO: 512 - DNA, 23 bp, synthetic construct
  source 1..23: mol_type = other DNA
ggactgaaaataaaaactctgag

SEQ ID NO: 513 - DNA, 23 bp, synthetic construct
  source 1..23: mol_type = other DNA
ggacagaaaacaaaaactctaca

SEQ ID NO: 514 - DNA, 24 bp, synthetic construct
  source 1..24: mol_type = other DNA
tttggagaaaatatgggttgaggt

SEQ ID NO: 515 - DNA, 23 bp, synthetic construct
  source 1..23: mol_type = other DNA
gggtggagttgggtataaaagag

SEQ ID NO: 516 - DNA, 23 bp, synthetic construct
  source 1..23: mol_type = other DNA
gggatgagttgggtataaaagat

SEQ ID NO: 517 - DNA, 22 bp, synthetic construct
  source 1..22: mol_type = other DNA
ggatgagttgggtataaaagat

SEQ ID NO: 518 - DNA, 23 bp, synthetic construct
  source 1..23: mol_type = other DNA
gggttgagttgggaataaaagag

SEQ ID NO: 519 - DNA, 23 bp, synthetic construct
  source 1..23: mol_type = other DNA
ggatagagttgggtataaagagg

SEQ ID NO: 520 - DNA, 22 bp, synthetic construct
  source 1..22: mol_type = other DNA
ggatagagttgggtataaagag

SEQ ID NO: 521 - DNA, 23 bp, synthetic construct
  source 1..23: mol_type = other DNA
gggtggagtggggtataaaagaa

SEQ ID NO: 522 - DNA, 23 bp, synthetic construct
  source 1..23: mol_type = other DNA
ggatggagttggatataaaagaa